(19) United States
(12) Patent Application Publication
(10) Pub. No.: US 2024/0351988 A1
(43) Pub. Date: Oct. 24, 2024

(54) ARYL HYDROCARBON RECEPTOR MODULATORS AND THEIR USE IN THE TREATMENT OF DISEASES AND DISORDERS

(71) Applicants: Allianthera (Suzhou) Biopharmaceutical Co., Ltd., Jiangsu (CN); Allianthera Boston Inc., Natick, MA (US)

(72) Inventors: Jinshan Chen, Madison, CT (US); Rajiv Chopra, Andover, MA (US); Jiamin Gu, Jiangsu (CN)

(21) Appl. No.: 18/685,378

(22) PCT Filed: Sep. 14, 2022
(86) PCT No.: PCT/US22/43403

§ 371 Date: Feb. 21, 2024

Related U.S. Application Data
(60) Provisional application No. 63/281,898, filed on Nov. 22, 2021.

(30) Foreign Application Priority Data
Sep. 14, 2021 (WO) PCT/CN21/118095
Mar. 4, 2022 (WO) PCT/CN22/079169

(51) Int. Cl. C07D 237/24; A61K 31/496; A61K 31/50; A61K 31/501; A61K 31/506; A61K 31/517; A61K 31/519; A61K 31/55; A61K 31/551; A61K 39/395; A61P 35/00; C07D 401/12; C07D 401/14; C07D 403/04; C07D 403/12; C07D 403/14; C07D 405/12; C07D 405/14; C07D 413/14; C07D 417/14; C07D 471/04

(52) U.S. Cl. CPC C07D 237/24; A61K 31/496; A61K 31/50; A61K 31/501; A61K 31/506; A61K 31/517; A61K 31/519; A61K 31/55; A61K 31/551; A61K 39/3955; A61P 35/00; C07D 401/12; C07D 401/14; C07D 403/04; C07D 403/12; C07D 403/14; C07D 405/12; C07D 405/14; C07D 413/14; C07D 417/14; C07D 471/04

(57) ABSTRACT

Disclosed herein are compounds which can act modulators of the aryl hydrocarbon receptor (AHR). Further disclosed herein are methods for treating diseases and disorders, such as cancer and viral infections, using the compounds disclosed herein.

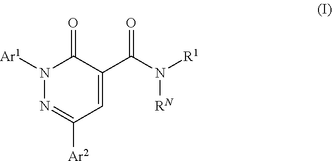

### FIELD OF THE INVENTION

This disclosure relates to compounds that are useful as aryl hydrocarbon receptor (AHR) activity modulators, pharmaceutical formulations thereof, and methods of using the compounds to treat diseases and disorders, such as cancer.

### DESCRIPTION OF RELATED TECHNOLOGY

The aryl hydrocarbon receptor (AHR) is a ligand-dependent transcription factor that regulates gene expression in a variety of cells, such as epithelial and immune cells. Emerging evidence suggests that AHR plays a role in the initiation, promotion, progression, invasion, and metastasis of cancer cells. Various tumor types and tumor cell lines show high AHR expression, suggesting that AHR is activated in tumors and facilitates their growth. Immune evasion is gaining recognition as a hallmark feature of cancer. A connection between the AHR and immune system has been identified, and AHR has been suggested as an immunosuppressive effector on different types of immune cells. Certain cancers can escape immune recognition via AHR signaling pathways.

Overall, modulating AHR activity in cancer cells, immune cells, stromal cells, fibroblasts, and endothelial cells within a tumor can slowdown the progression of the disease.

AHR is expressed at high levels and is chronically active in blood tumors, such as T-cell leukemia and lymphoma, as well as in solid tumors, such as glioblastoma, ovarian cancer, lung cancer, liver cancer, and head and neck carcinomas. It has been suggested that detection of AHR activity in the tumor microenvironment can serve as a potent diagnostic indicator for tumor aggressiveness. Depending on the cancer type, two types of results are associated with AHR activity and the prognosis. There is evidence that, in hormone-dependent breast cancers, AHR activation is associated with attenuated aggressiveness and a better prognosis. In contrast, higher AHR activity has been suggested as being correlated with increased aggressiveness and a poor prognosis in non-small-cell lung cancer.

Recent reports have also demonstrated the role of AHR as a modulator of the intrinsic, innate and adaptive immune response to viral infections. The role of AHR activation has been recognized as influencing host resistance to infections by a variety of viruses, including influenza, coronaviruses (such as SARS-CoV-1, SARS-CoV-2, and MERS-CoV), flaviviruses (such as Zika virus), retroviruses (such as HIV), and herpesviruses.

As such, there is a need for novel compounds that modulate AHR activity to provide new and effective therapies for diseases and disorders, such as cancer and viral infections.

### SUMMARY

One aspect of the disclosure provides a compound, or pharmaceutically acceptable salt thereof, having a structure of Formula (I):

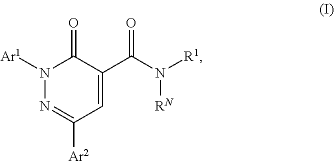

wherein each R^{N} is independently H or C₁₋₆alkyl; R¹ is H, C₁₋₆alkyl, C₁₋₆alkylene-NR^{N}R^{N}, C₁₋₆alkylene-O—C₁₋₆alkyl, C₁₋₆alkylene-C(O)R², C₁₋₆alkylene-NR^{N}—C(O)—C₁₋₃alkyl, C₃₋₈cycloalkyl, or 4-12 membered heterocyclyl, wherein 1-3 ring atoms are selected from O, N, and S, and the cycloalkyl or heterocyclyl is optionally substituted with 1 or 2 C₁₋₆alkyl, C(O)—C₁₋₆alkyl, and ═O; R² is OH, O—C₁₋₆alkyl, or NR^{N}R^{N}; Ar¹ is C₆₋₁₀aryl or 5-10 membered heteroaryl, wherein 1-3 ring atoms are selected from O, N, and S, and Ar¹ is optionally substituted with 1 or 2 R³; each R³ is independently halo, C₁₋₆alkyl, C₁₋₆haloalkyl, or C₃₋₆cycloalkyl; Ar² is C₅₋₈cycloakly, C₅₋₈cycloalkenyl, C₆₋₁₀aryl, or 5-12 membered heterocyclyl or heteroaryl, wherein 1-3 ring atoms are selected from O, N, and S, and Ar² is optionally substituted with 1, 2, or 3 R⁴; each R⁴ is independently halo, OH, ═O, CN, C₁₋₆alkyl, C₁₋₆haloalkyl, C₁₋₆hydroxyalkyl, C₁₋₆alkoxy, C₁₋₆haloalkoxy, NR^{N}R^{N}, CONR^{N}R^{N}, COOH, COO—C₁₋₆alkyl, C(O)—C₁₋₆alkyl, SO₂—C₁₋₆alkyl, C₃₋₈cycloalkyl, 4-12 membered heterocyclyl or 5-12 membered heteroaryl, wherein 1-3 ring atoms are selected from O, N, and S, or phenyl, and the heterocyclyl, heteroaryl, or phenyl is substituted with 0, 1, or 2 substituents independently selected from halo and C₁₋₆alkyl, or R¹ can be C₂₋₆hydroxyalkyl when Ar² is (1) C₅₋₈cycloakly, C₅₋₈cycloalkenyl, C₁₀aryl, 4-12-membered heterocyclyl, or 5- or 7-12 membered heteroaryl and Ar² is optionally substituted with 1, 2, or 3 R⁴; or (2) phenyl or 6-membered heteroaryl and Ar² is substituted with at least one R⁴ selected from CONR^{N}R^{N}, COOH, COO—C₁₋₆alkyl, C(O)—C₁₋₆alkyl, SO₂—C₁₋₆alkyl, C₃₋₆cycloalkyl, 4-12 membered heterocyclyl, 5-12 membered heteroaryl, and phenyl, and the phenyl, heterocyclyl or heteroaryl is substituted with 0, 1, or 2 substituents independently selected from halo and C₁₋₆alkyl.

In some cases, each R^{N} is independently H or methyl. In some cases, R¹ is H or C₁₋₆alkyl. In some cases, R¹ is methyl. In some cases, R¹ is C₁₋₆alkylene-NR^{N}R^{N} or C₁₋₆alkylene-O—C₁₋₆alkyl. In some cases, R¹ is

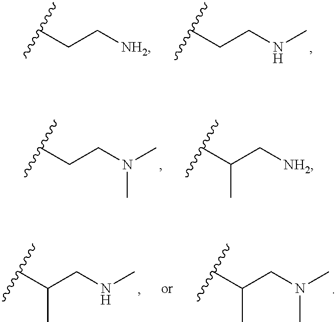

In some cases, R¹ is

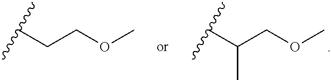

In some cases, R¹ is

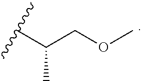 [SMILES: *[C@@H](C)COC]

In some cases, R¹ is C₁₋₆alkylene-C(O)R² or C₁₋₆alkylene-NR^{N}—C(O)CH₃. In some cases, R² is OH. In some cases, R² is NH₂, NHCH₃, or N(CH₃)₂. In some cases, R¹ is

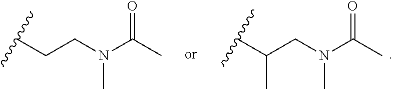

In some cases, R¹ is

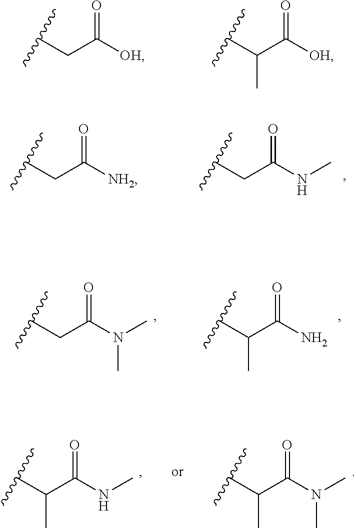

In some cases, R¹ is heterocyclyl comprising 4-12 total ring atoms, wherein 1-3 of the ring atoms are selected from O, N, and S. In some cases, the heterocyclyl is unsubstituted. In some cases, the heterocyclyl is substituted with 1 or 2 C₁₋₆alkyl, C(O)—C₁₋₆alkyl, or ═O. In some cases, R¹ is

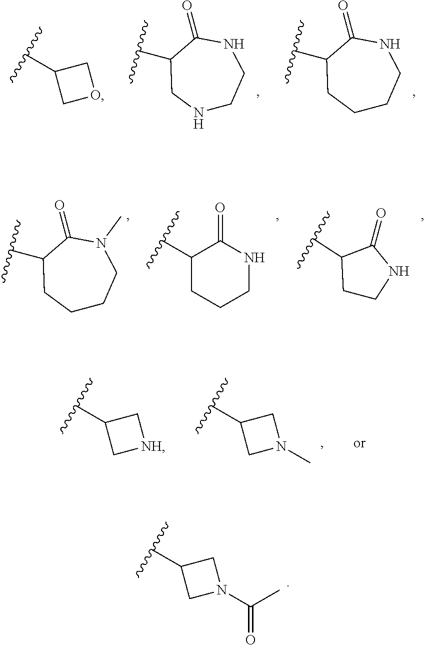

In some cases, R¹ is

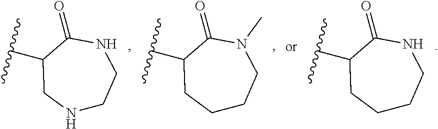

In some cases, R¹ is C₂₋₆alkylene-C(O)—NR^{N}R^{N}. In some cases, R¹ is

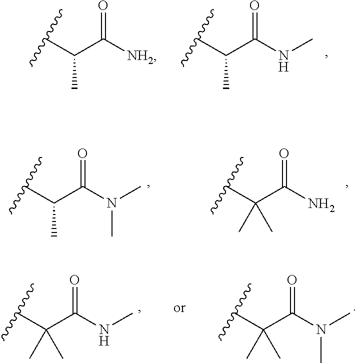

In some cases, R¹ is C₂₋₆hydroxyalkyl, e.g.,

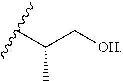 [SMILES: *[C@@H](C)CO]

In some cases, Ar¹ is C₆₋₁₀aryl. In some cases, Ar¹ is phenyl. In some cases, Ar¹ is 5-10 membered heteroaryl. In some cases, Ar¹ is unsubstituted. In some cases, Ar¹ is

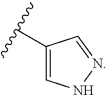 [SMILES: *c1cn[nH]c1]

In some cases, Ar¹ is substituted with 1 or 2 R³. In some cases, R³ is fluoro, chloro, methyl, ethyl, isopropyl, cyclopropyl, or trifluromethyl. In some cases, Ar¹ is

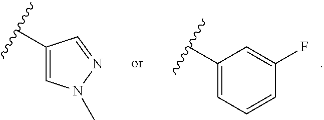

In some cases, Ar¹ is

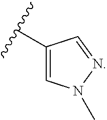 [SMILES: *c1cnn(C)c1]

In some cases, Ar² is C₆₋₁₀aryl. In some cases, Ar² is phenyl. In some cases, Ar² is 5-12 membered heteroaryl. In some cases, Ar² is C₅₋₈cycloalkyl, C₅₋₈cycloalkenyl, or 5-12 membered heterocyclyl. In some cases, Ar² is unsubstituted. In some cases, Ar² is substituted with 1, 2, or 3 R⁴. In some cases, Ar² is substituted with 1 or 2 R⁴. In some cases, at least one R⁴ is halo. In some cases, at least one R⁴ is chloro. In some cases, Ar² is

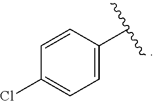 [SMILES: *c1ccc(Cl)cc1]

In some cases, at least one R⁴ is fluoro, chloro, OH, CN, CH₃, OCH₃, CF₃, OCF₃, CHF₂, CH₂F, OCHF₂, NH₂, N(CH₃)₂, CONH₂, COOH, SO₂CH₃, cyclopropyl, or morpholino. In some cases, at least one R⁴ selected from CONR^{N}R^{N}, COOH, COO—C₁₋₆alkyl, C(O)—C₁₋₆alkyl, SO₂—C₁₋₆alkyl, C₃₋₈cycloalkyl, 4-12 membered heterocyclyl, 5-12 membered heteroaryl, and phenyl, and the phenyl, heterocyclyl or heteroaryl is substituted with 0, 1, or 2 substituents independently selected from halo and C₁₋₆alkyl. In some cases, Ar² is phenyl, pyridyl, pyrimidinyl, pyrazolyl, or triazolyl, and at least one R⁴ is phenyl or halo substituted-phenyl.

In some cases, Ar² is

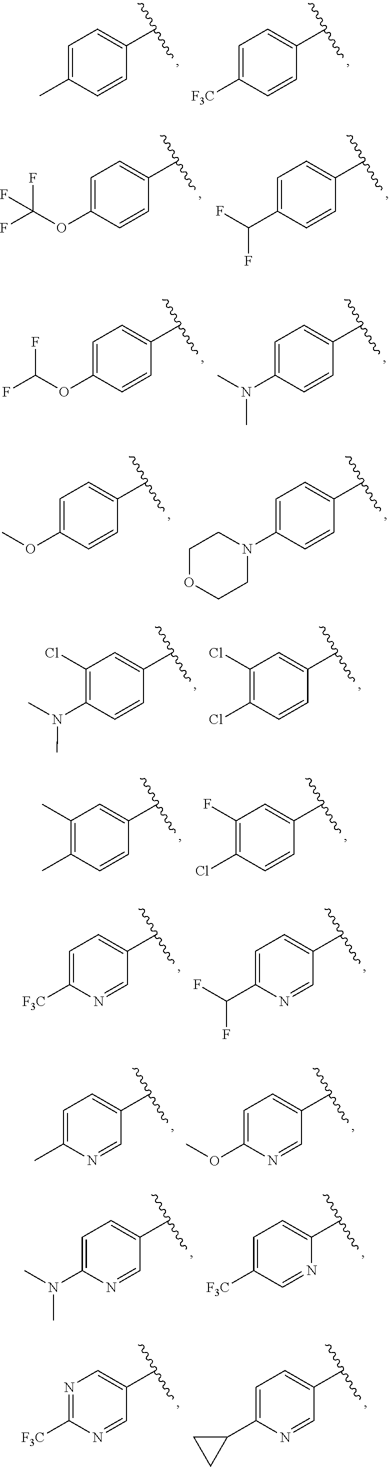

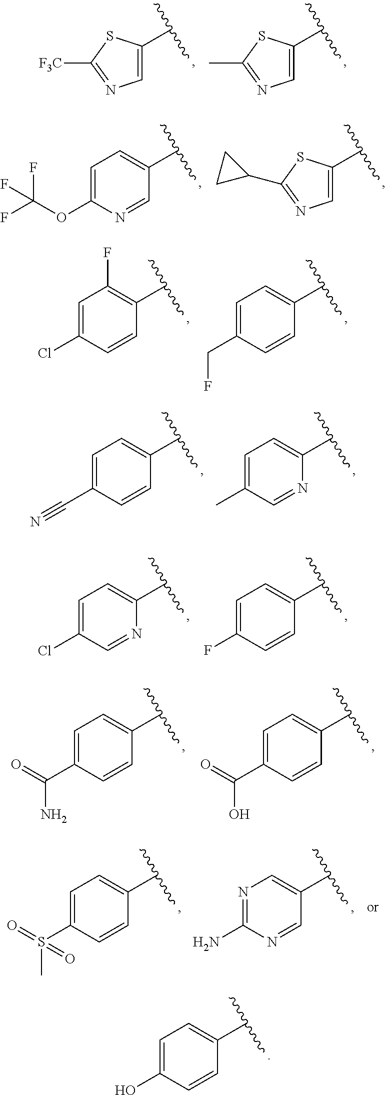

In some cases, Ar² is

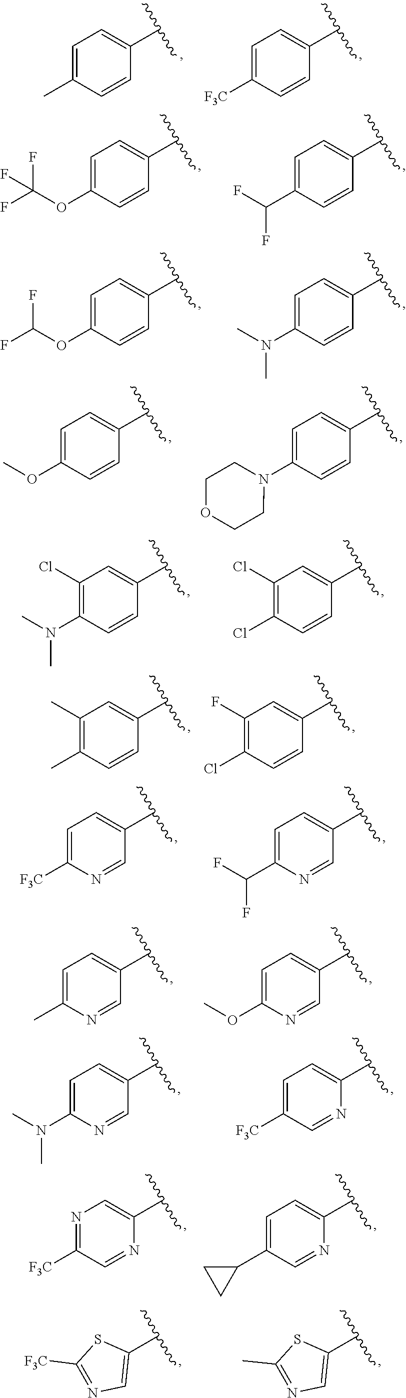

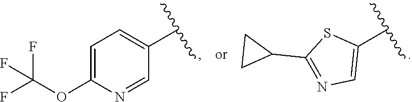 [SMILES: *c1ccc(OC(F)(F)F)nc1 ; *c1cnc(C2CC2)s1]

In some cases, Ar² is

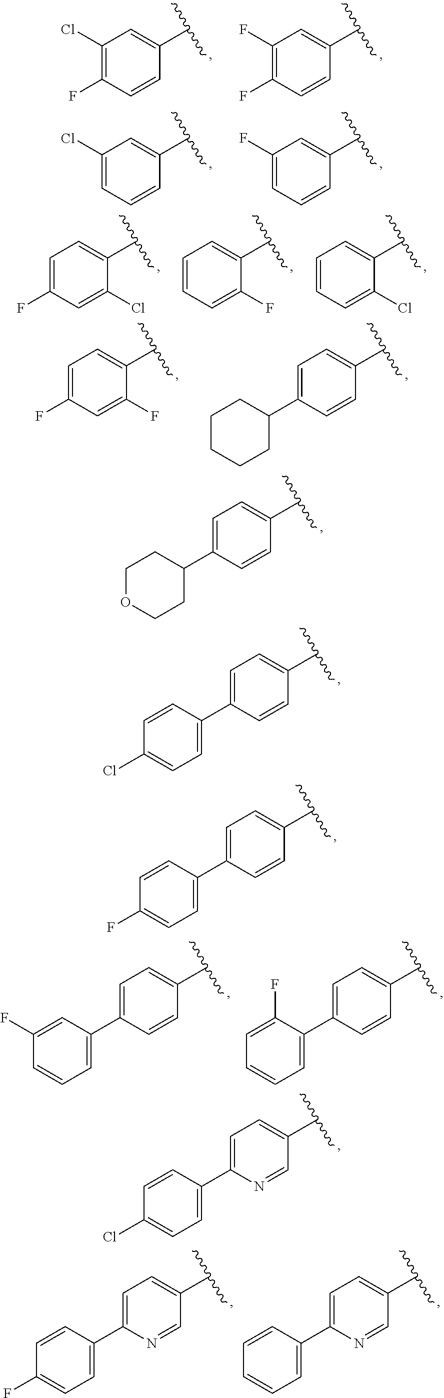

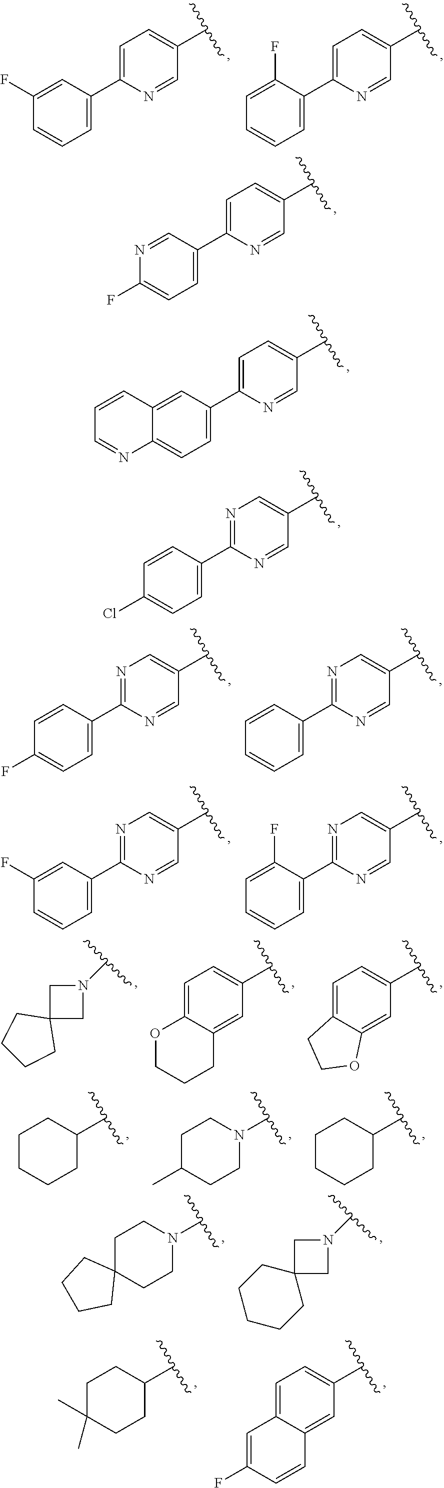

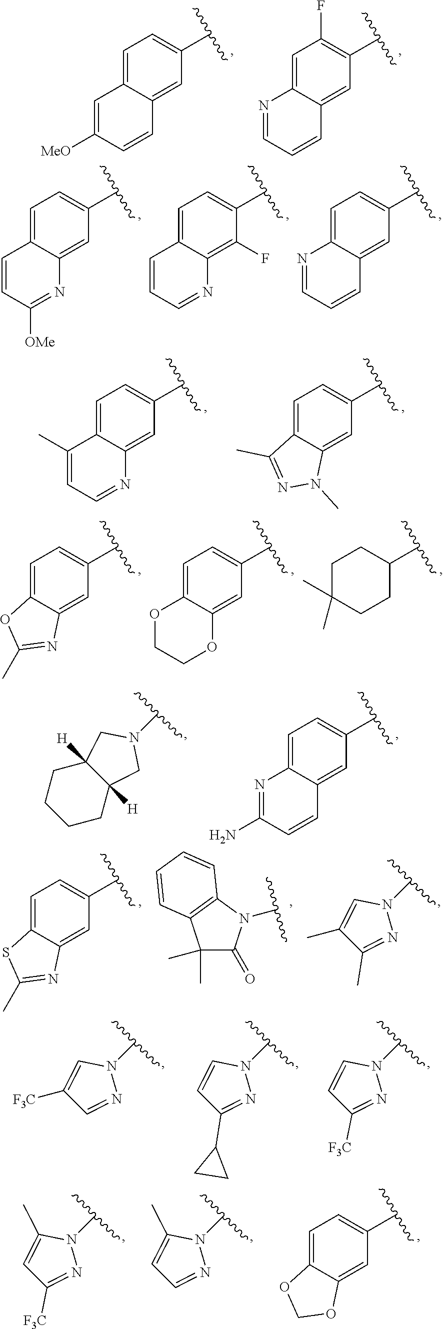

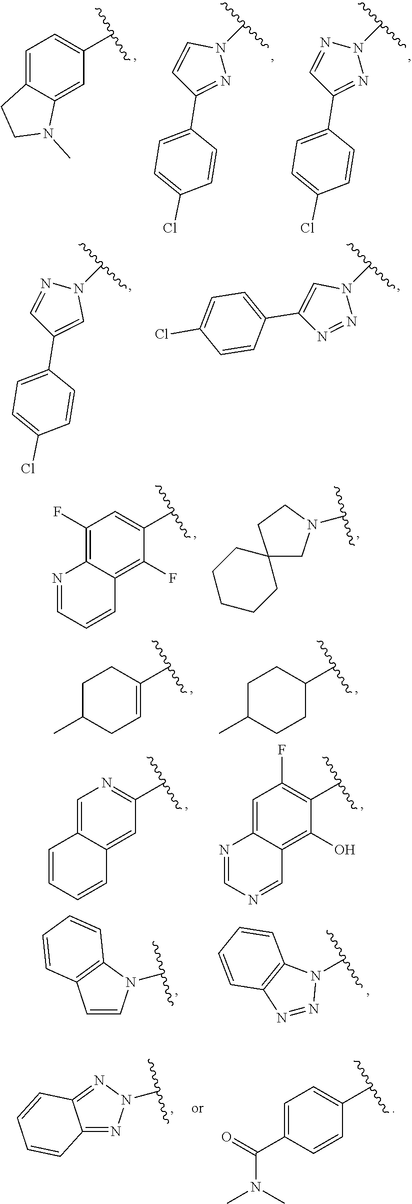

In another aspect, the disclosure provides a compound as listed in Table 1, or a pharmaceutically acceptable salt thereof.

In a further aspect, the disclosure provides a pharmaceutical formulation comprising the compound or salt of the disclosure (e.g., compounds of Formula (I), Table 1, and salts thereof) and a pharmaceutically acceptable excipient.

Yet another aspect of the disclosure provides a method for modulating aryl hydrocarbon receptor (AHR) activity in a subject, comprising contacting the AHR with a compound or salt of the disclosure (e.g., compounds of Formula (I), Table 1, and salts thereof) or a pharmaceutical composition thereof, as disclosed herein.

Another aspect of the disclosure provides a method for treating or preventing a disease or disorder in a subject, comprising administering to the subject a therapeutically effective amount of a compound or salt of the disclosure (e.g., compounds of Formula (I), Table 1, and salts thereof), or the pharmaceutical composition thereof, as disclosed herein. In some cases, the disease or disorder is cancer, a viral infection, or pulmonary arterial hypertension (PAH). In some cases, the viral infection is flavivirus infection or coronavirus infection. In some cases, the viral infection is flavivirus infection. In some cases, the flavivirus infection is Zika virus infection.

In some cases, the viral infection is coronavirus infection. In some cases, the coronavirus infection is severe acute respiratory syndrome (SARS), Middle East respiratory syndrome (MERS), or Coronavirus disease 2019 (COVID-19). In some cases, the coronavirus infection is Coronavirus disease 2019 (COVID-19). In some cases, the disease or disorder is cancer. In some cases, the cancer is a liquid or solid tumor, hematological cancer, lymphoma, myeloma, leukemia, sarcoma, eye cancer, thyroid cancer, parathyroid cancer, neurological cancer, skin cancer, breast cancer, uterine cancer, endometrial cancer, prostate cancer, colorectal cancer, lung cancer, head and neck cancer, gastrointestinal cancer, liver cancer, pancreatic cancer, genitourinary cancer, bone cancer, renal cancer, or vascular cancer. In some cases, the method further comprises administration of a therapeutic agent. In some cases, the therapeutic agent is an immune checkpoint inhibitor. In some cases, the immune checkpoint is PD1 or PDL1. In some cases, the immune checkpoint inhibitor is pembrolizumab, nivolumad, cemiplimab, atezolizumab, dostarlimab, durvalumab, avelumab, or a combination thereof.

Another aspect of the disclosure relates to use of a compound or salt disclosed herein (e.g., compounds of Formula (I), Table 1, and salts thereof), or a pharmaceutical composition disclosed herein for modulating AHR activity in a subject. A further aspect of the disclosure relates to use of a compound disclosed herein (e.g., compounds of Formula (I), Table 1, and salts thereof), or a pharmaceutical composition disclosed herein for treating or preventing a disease or disorder in a subject.

Further provided are compounds of Formula (A) or (B):

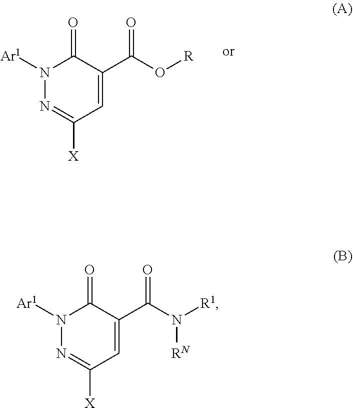

wherein Ar¹ is C₆₋₁₀aryl or 5-10 membered heteroaryl, wherein 1-3 ring atoms are selected from O, N, and S, and Ar¹ is optionally substituted with 1 or 2 R³; each R³ is independently halo, C₁₋₆alkyl, C₁₋₆haloalkyl, or C₃₋₆cycloalkyl; R¹ is H, C₁₋₆alkyl, C₁₋₆alkylene-NR^{N}R^{N}, C₁₋₆alkylene-O—C₁₋₆alkyl, C₁₋₆alkylene-C(O)R², C₁₋₆alkylene-NR^{N}—C(O)—C₁₋₃alkyl, C₃₋₈cycloalkyl, or 4-12 membered heterocyclyl, wherein 1-3 ring atoms are selected from O, N, and S, and the cycloalkyl or heterocyclyl is optionally substituted with 1 or 2 C₁₋₆alkyl, C(O)—C₁₋₆alkyl, and ═O; R² is OH, O—C₁₋₆alkyl, or NR^{N}R^{N}; each R^{N} is independently H or C₁₋₆alkyl; X is a leaving group; and R is H, C₁₋₆alkyl, or phenyl. In some cases, X is chloro, fluoro, mesyl, tosyl, or triflyl.

Also provided are methods of preparing a compound or salt of Formula (I) comprising admixing a compound of Formula (B) as disclosed herein with Ar²-boronic acid or Ar²-boronic ester in the presence of a catalyst to form the compound of Formula (I). Further provides are methods of preparing a compound or salt of Formula (I), comprising admixing a compound of Formula (A) with Ar²-boronic acid or Ar²-boronic ester in the presence of a catalyst to form an intermediate then converting the —OR moiety of the intermediate to a —N(R^{N})R¹ moiety to form the compound of Formula (I). In various cases, the catalyst comprises a palladium catalyst. Also provided are methods of preparing the compound or salt of Formula (I), comprising admixing the compound of Formula (B) with an amine nucleophile, optionally in the presence of a catalyst, to form an aromatic intermediate, then reacting the aromatic intermediate with a reagent to form the compound of Formula (I). Further provided are methods of preparing the compound or salt of Formula (I), comprising (i) admixing the compound of Formula (A) with an amine nucleophile, optionally in the presence of a catalyst, to form an aromatic intermediate, (ii) reacting the aromatic intermediate with a reagent to form an ester intermediate, and (iii) converting the —OR moiety of the ester intermediate to a —N(R^{N})R¹ moiety to form the compound of Formula (I).

Further aspects and advantages will be apparent to those of ordinary skill in the art from a review of the following detailed description, taken in conjunction with the drawings. While the compounds and methods disclosed herein are susceptible of embodiments in various forms, the description hereafter includes specific embodiments with the understanding that the disclosure is illustrative, and is not intended to limit the invention to the specific embodiments described herein.

### BRIEF DESCRIPTIONS OF THE FIGURES

FIG. 1 shows tumor volume over time of mice having CT26 tumor cells dosed with (a) vehicle; (b) anti-PD1 antibody; (c) Compound Example 184; or (d) combination of Compound Example 184 and anti-PD1 antibody.

FIG. 2 shows the survival of mice having CT26 tumor cells and dosed with (a) vehicle; (b) anti-PD1 antibody; (c) Compound Example 184; or (d) combination of Compound Example 184 and anti-PD1 antibody.

### DETAILED DESCRIPTION

Provided herein are compounds of Formula (I):

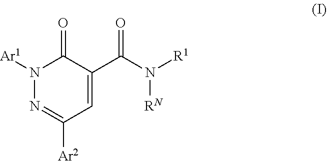

wherein Ar¹, Ar², R¹, and R^{N} are as described herein, which can function as modulators of AHR activity. For example, the compounds can increase or decrease AHR activity.

The compounds disclosed herein are useful for targeting and selectively inhibiting AHR. It has been found that multiple tumor types have high levels of AHR signaling as determined by an AHR-gene signature. The high level of AHR activation caused by elevated levels of kynurenine and other ligands, as well as its role in driving an immune suppressive tumor microenvironment (TME), make AHR an attractive therapeutic target in multiple cancer types.

Many viral infections have also been found to involve AHR signaling. AHR is implicated in regulating the immune response, and AHR activation has been associated with adaptive immune response impairment and poor health outcomes in a variety of viral infections (e.g., influenza). Inhibition of AHR has also been associated with diminished production of viral particles in vivo (e.g., Dengue fever). AHR appears to play a role in controlling lipid biogenesis, a hallmark of Hepatitis C (HCV) infection. The role of AHR in the regulation of the host immune response against many viruses, such as the examples given above, make modulation of AHR an attractive therapeutic target for viral infections.

AHR modulation has also been implicated in treating pulmonary arterial hypertension (PAH).

Accordingly, in some cases, the present disclosure provides compounds useful for treating a disease or disorder, e.g., cancer, a viral infection, or PAH, in a subject. Also provided are methods of treating a disease or disorder, e.g., cancer, a viral infection, or PAH, in a subject, comprising administering to the subject a therapeutically effective amount of a compound disclosed herein, e.g., a compound of Formula (I) or Table 1, or a pharmaceutically acceptable salt thereof. Further provided are pharmaceutical compositions comprising a compound disclosed herein, e.g., a compound of Formula (I) or Table 1, or a pharmaceutically acceptable salt thereof, and methods of using such pharmaceutical compositions in methods of treating a disease or disorder, e.g., cancer, a viral infection, or PAH.

### Compounds of the Disclosure

In one aspect, disclosed herein are compounds having a structure of Formula (I):

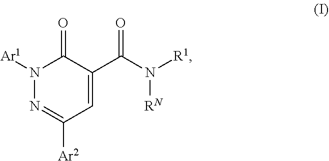

wherein
each R^{N} is independently H or C₁₋₆alkyl;
R¹ is H, C₁₋₆alkyl, C₁₋₆alkylene-NR^{N}R^{N}, C₁₋₆alkylene-O—C₁₋₆alkyl, C₁₋₆alkylene-C(O)R², C₁₋₆alkylene-NR^{N}—C(O)—C₁₋₃alkyl, C₃₋₈cycloalkyl, or 4-12 membered heterocyclyl, wherein 1-3 ring atoms are selected from O, N, and S, and the cycloalkyl or heterocyclyl is optionally substituted with 1 or 2 C₁₋₆alkyl, C(O)—C₁₋₆alkyl, and ═O;
R² is OH, O—C₁₋₆alkyl, or NR^{N}R^{N};
Ar¹ is C₆₋₁₀aryl or 5-10 membered heteroaryl, wherein 1-3 ring atoms are selected from O, N, and S, and Ar¹ is optionally substituted with 1 or 2 R³;
each R³ is independently halo, C₁₋₆alkyl, C₁₋₆haloalkyl, or C₃₋₆cycloalkyl;
Ar² is C₅₋₈cycloakly, C₅₋₈cycloalkenyl, C₆₋₁₀aryl, or 5-12 membered heterocyclyl or heteroaryl, wherein 1-3 ring atoms are selected from O, N, and S, and Ar² is optionally substituted with 1, 2, or 3 R⁴; each R⁴ is independently halo, OH, ═O, CN, C₁₋₆alkyl, C₁₋₆haloalkyl, C₁₋₆hydroxyalkyl, C₁₋₆alkoxy, C₁₋₆haloalkoxy, NR^{N}R^{N}, CONR^{N}R^{N}, COOH, COO—C₁₋₆alkyl, C(O)—C₁₋₆alkyl, SO₂—C₁₋₆alkyl, C₃₋₆cycloalkyl, 4-12 membered heterocyclyl or 5-12 membered heteroaryl, wherein 1-3 ring atoms are selected from O, N, and S, or phenyl, and the heterocyclyl, heteroaryl, or phenyl is substituted with 0, 1, or 2 substituents independently selected from halo and C₁₋₆alkyl,
   or R¹ can be C₂₋₆hydroxyalkyl when Ar² is (1) C₅₋₈cycloakly, C₅₋₈cycloalkenyl, C₁₀aryl, 4-12-membered heterocyclyl, or 5- or 7-12 membered heteroaryl and Ar² is optionally substituted with 1, 2, or 3 R⁴; or (2) phenyl or 6-membered heteroaryl and Ar² is substituted with at least one R⁴ selected from CONR^{N}R^{N}, COOH, COO—C₁₋₆alkyl, C(O)—C₁₋₆alkyl, S02-C₁₋₆alkyl, C₃₋₈cycloalkyl, 4-12 membered heterocyclyl, 5-12 membered heteroaryl, and phenyl, and the phenyl, heterocyclyl or heteroaryl is substituted with 0, 1, or 2 substituents independently selected from halo and C₁₋₆alkyl.

In some cases, R^{N} is H. In some cases, R^{N} is C₁₋₆alkyl. In some cases, R^{N} is methyl.

In some cases, R¹ is H. In some cases, R¹ is C₁₋₆alkyl. In some cases, R¹ is methyl, ethyl, propyl, or isopropyl. In some cases, R¹ is methyl.

In some cases, R¹ is C₁₋₆alkylene-NR^{N}R^{N} or C₁₋₆alkylene-O—C₁₋₆alkyl. In some cases, R¹ is C₁₋₆alkylene-NR^{N}R^{N}. In some cases, R¹ is C₂₋₃alkylene-NR^{N}R^{N}. In some cases, R¹ is C₂alkylene-NR^{N}R^{N}. In some cases, R¹ is C₃alkylene-NR^{N}R^{N}. In some cases, each R^{N} independently is H or CH₃. In some cases, each R^{N} is H. In some cases, each R^{N} is CH₃. In some cases, one R^{N} is H and one R^{N} is CH₃. In some cases, R¹ is

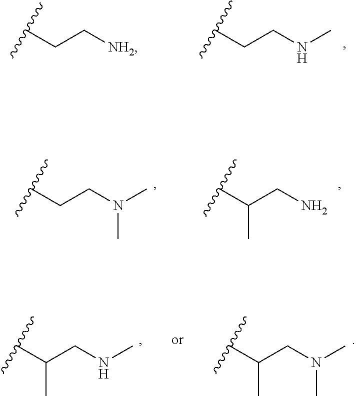

In some cases, R¹ is C₁₋₆alkylene-O—C₁₋₆alkyl. In some cases, R¹ is C₂₋₃alkylene-O—C₁alkyl. In some cases, R¹ is C₂alkylene-O—C₁alkyl. In some cases, R¹ is C₃alkylene-O—C₁alkyl. In some cases, R¹ is

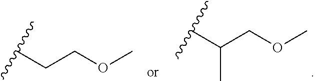

In some cases, R¹ is

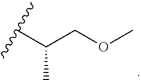 [SMILES: *[C@@H](C)COC]

In some cases, R¹ is H, C₁₋₆alkyl, C₁₋₆alkylene-NR^{N}R^{N}, C₁₋₆alkylene-O—C₁₋₆alkyl, C₁₋₆alkylene-C(O)R², C₁₋₆alkylene-NR^{N}—C(O)—C₁₋₃alkyl, or heterocyclyl comprising 4-12 total ring atoms, wherein 1-3 of the ring atoms are selected from O, N, and S and the heterocyclyl is optionally substituted with 1 or 2 C₁₋₆alkyl, C(O)—C₁₋₆alkyl, or ═O. In some cases, R¹ is C₁₋₆alkylene-C(O)R² or C₁₋₆alkylene-NR^{N}—C(O)—C₁₋₃alkyl. In some cases, R¹ is C₁₋₆alkylene-C(O)R² or C₁₋₆alkylene-NR^{N}—C(O)—CH₃. In some cases, R¹ is C₂₋₃alkylene-C(O)R² or C₂₋₃alkylene-NR^{N}.

C(O)—C₁₋₃alkyl. In some cases, R¹ is C₂₋₃alkylene-C(O)R² or C₂₋₃alkylene-NR^{N}—C(O)—CH₃. In some cases, R¹ is C₁₋₆alkylene-NR^{N}—C(O)—C₁₋₃alkyl. In some cases, R¹ is C₂₋₃alkylene-NR^{N}—C(O)—C₁₋₃alkyl. In some cases, R¹ is C₂alkylene-NR^{N}—C(O)—C₁₋₃alkyl. In some cases, R¹ is C₃alkylene-NR^{N}—C(O)—C₁₋₃alkyl. In some cases, R¹ is C₁₋₆alkylene-NR^{N}—C(O)—CH₃. In some cases, R¹ is C₂₋₃alkylene-NR^{N}—C(O)—CH₃. In some cases, R¹ is C₂alkylene-NR^{N}—C(O)—CH₃. In some cases, R¹ is C₃alkylene-NR^{N}—C(O)—CH₃. In some cases, R¹ is C₁₋₆alkylene-C(O)R². In some cases, R¹ is C₁₋₃alkylene-C(O)R². In some cases, R¹ is C₁alkylene-C(O)R². In some cases, R¹ is C₂alkylene-C(O)R². In some cases, R¹ is C₃alkylene-C(O)R². In some cases, R² is OH. In some cases, R² is 0-C₁₋₆alkyl. In some cases, R² is O—CH₃. In some cases, R^{N} is H. In some cases, R^{N} is CH₃. In some cases, R² is NR^{N}R^{N}. In some cases, R² is NH₂. In some cases, R² is NHCH₃. In some cases, R² is N(CH₃)₂. In some cases, R¹ is

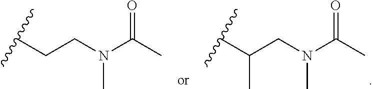

In some cases, R¹ is

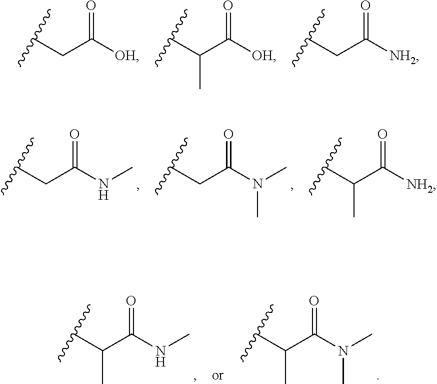

In some cases, In some cases, R¹ is C₃₋₆cycloalkyl optionally substituted with 1 or 2 C₁₋₆alkyl, C(O)—C₁₋₆ alkyl, or ═O. In some cases, R¹ is unsubstituted C₃₋₆cycloalkyl. In some cases, R¹ is C₃₋₆cycloalkyl substituted with 1 or 2 C₁₋₆alkyl, C(O)—C₁₋₆alkyl, or ═O. In some cases, R¹ is C₃₋₆cycloalkyl substituted with 1 or 2=O. In some cases, R¹ is C₃₋₆cycloalkyl substituted with 1=O, In some cases, R¹ is

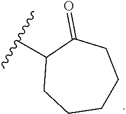 [SMILES: *C1CCCCCC1=O]

In some cases, R¹ is

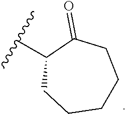 [SMILES: *[C@H]1CCCCCC1=O]

In some cases, R¹ is heterocyclyl comprising 4-12 total ring atoms, wherein 1-3 of the ring atoms are selected from O, N, and S and the heterocyclyl is optionally substituted with 1 or 2 C₁₋₆alkyl, C(O)—C₁₋₆alkyl, or ═O. In some cases, R¹ is heterocyclyl comprising 4-7 total ring atoms. In some cases, R¹ is heterocyclyl comprising 4 total ring atoms. In some cases, R¹ is heterocyclyl comprising 5 total ring atoms. In some cases, R¹ is heterocyclyl comprising 6 total ring atoms. In some cases, R¹ is heterocyclyl comprising 7 total ring atoms. In some cases, heterocyclyl is oxetanyl, azetidinyl, diazepanyl (such as 1,4-diazepanyl), azepanyl, piperidinyl (such as 1-piperidinyl, 2-piperidinyl, 3-piperidinyl, or 4-piperidinyl), pyrrolidinyl (such as 1-pyrrolidinyl, 2-pyrrolidinyl, or 3-pyrrolidinyl), piperazinyl, pyrazolidinyl, imidazolidinyl, triazepanyl, oxazolidinyl, isoxazolidinyl, thiazolidinyl, isothiazolidinyl, oxazepanyl, thiazepanyl, tetrahydrofuranyl, tetrahydrothiophenyl, morpholino (such as 3-morpholino or 4-morpholino), thiomorpholino (such as 2-thiomorpholino, 3-thiomorpholino or 4-thiomorpholino), pyrrolidin-2-one, tetrahydropiperazinyl (such as 1-tetrahydropiperazinyl, 2-tetrahydropiperazinyl, or 3-tetrahydropiperazinyl), pyrazolinyl (such as 1-pyrazolinyl, 3-pyrazolinyl, 4-pyrazolinyl, or 5-pyrazolinyl), thiazolidinyl (such as 2-thiazolidinyl, 3-thiazolidinyl, or 4-thiazolidinyl), imidazolidinyl (such as 1-imidazolidinyl, 2-imidazolidinyl, 4-imidazolidinyl, or 5-imidazolidinyl), or 1,3-dihydro-imidazol-2-only. In some cases, heterocyclyl is oxetanyl, azetidinyl, diazepanyl (such as 1,4-diazepanyl), azepanyl, piperidinyl (such as 1-piperidinyl, 2-piperidinyl, 3-piperidinyl, or 4-piperidinyl), or pyrrolidinyl (such as 1-pyrrolidinyl, 2-pyrrolidinyl, or 3-pyrrolidinyl). In some cases, heterocyclyl is oxetanyl, azetidinyl, diazepanyl or azepanyl. In some cases, heterocyclyl is diazepanyl or azepanyl. In some cases, heterocyclyl is 1,4-diazepanyl or azepanyl. In some cases, heterocyclyl is 1,4-diazepanyl. In some cases, heterocyclyl is azepanyl. In some cases, R¹ is unsubstituted heterocyclyl. In some cases, R¹ is heterocyclyl substituted with 1 or 2 C₁₋₆alkyl, C(O)—C₁₋₆alkyl, or ═O. In some cases, R¹ is heterocyclyl substituted with 1 C₁₋₆alkyl, C(O)—C₁₋₆alkyl, or ═O. In some cases, R¹ is heterocyclyl substituted with 2 C₁₋₆alkyl, C(O)—C₁₋₆alkyl, or ═O. In some cases, R¹ is heterocyclyl substituted with C₁₋₆alkyl. In some cases, R¹ is heterocyclyl substituted with C(O)—C₁₋₆alkyl. In some cases, R¹ is heterocyclyl substituted with ═O. In some cases, R¹ is a lactam. In some cases, R¹ is

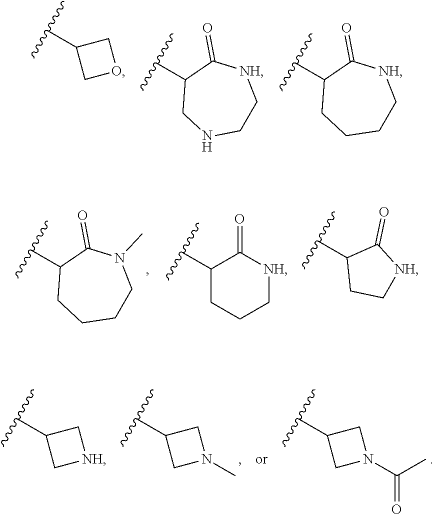

In some cases, R¹ is

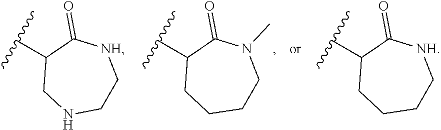

In some cases, R¹ is H, methyl,

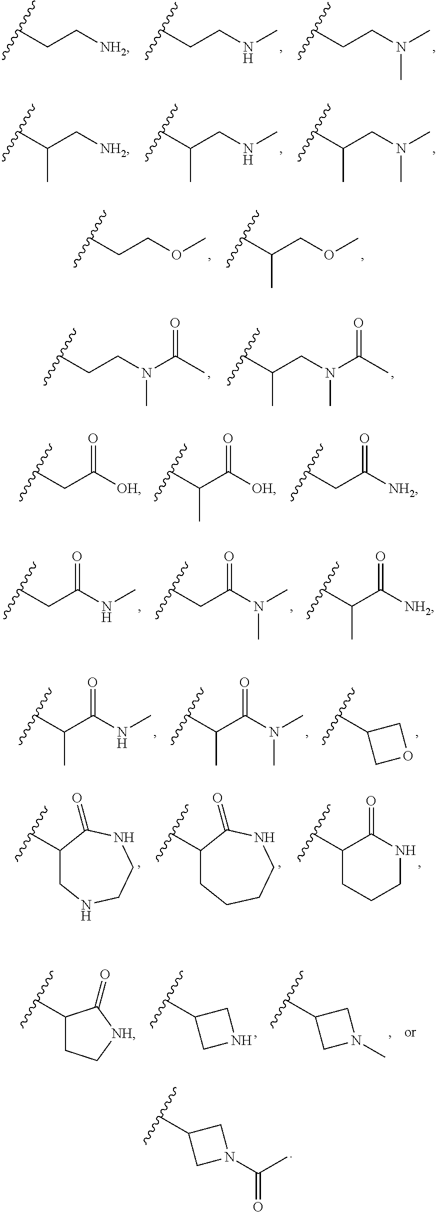

In some cases, R¹ is

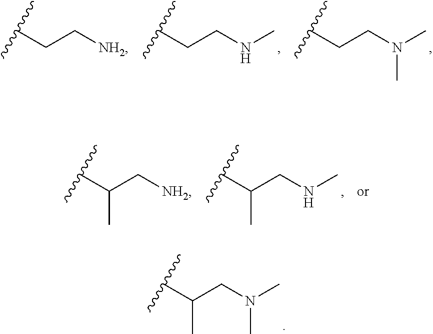

In some cases, R¹ is

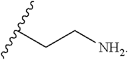 [SMILES: *CCN]

In some cases, R¹ is

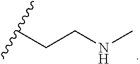 [SMILES: *CCNC]

In some cases, R¹ is

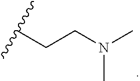 [SMILES: *CCN(C)C]

In some cases, R¹ is

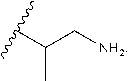 [SMILES: *C(C)CN]

In some cases, R¹ is

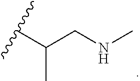 [SMILES: *C(C)CNC]

In some cases, R¹ is

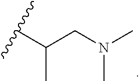 [SMILES: *C(C)CN(C)C]

In some oases, R¹ is

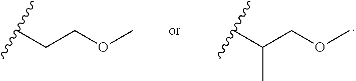

In some oases, R¹ is

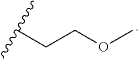 [SMILES: *CCOC]

In some oases, R¹ is

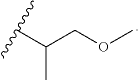 [SMILES: *C(C)COC]

In some oases, R¹ is

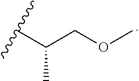 [SMILES: *[C@@H](C)COC]

In some cases, R¹ is

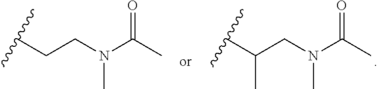

In some cases, R¹ is

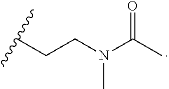 [SMILES: *CCN(C)C(C)=O]

In some cases, R¹ is

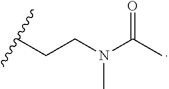 [SMILES: *CCN(C)C(C)=O]

In some cases, R¹ is

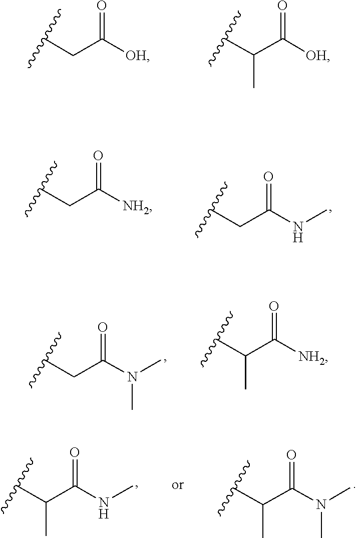

In some cases, R¹ is

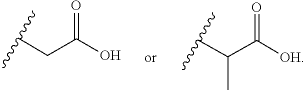

In some cases, R¹ is

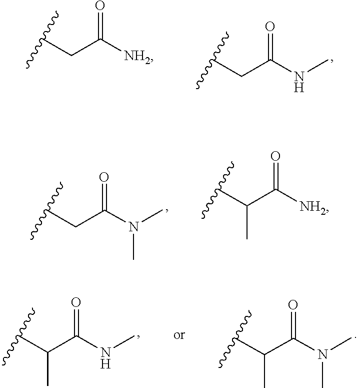

In some cases, R¹ is

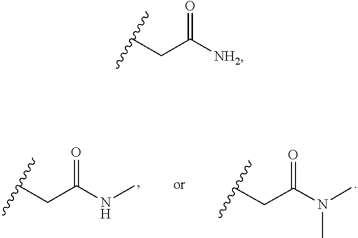

In some cases, R¹ is

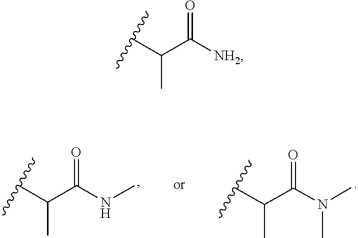

In some cases, R¹ is

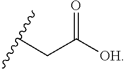 [SMILES: *CC(=O)O]

In some cases, R¹ is

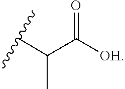 [SMILES: *C(C)C(=O)O]

In some cases, R¹ is

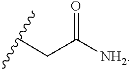 [SMILES: *CC(N)=O]

In some cases, R¹ is

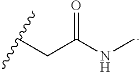 [SMILES: *CC(=O)NC]

In some cases, R¹ is

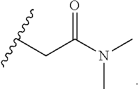 [SMILES: *CC(=O)N(C)C]

In some cases, R¹ is

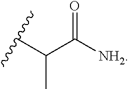 [SMILES: *C(C)C(N)=O]

In some cases, R¹ is

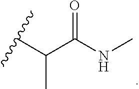 [SMILES: *C(C)C(=O)NC]

In some cases, R¹ is

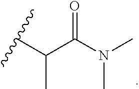 [SMILES: *C(C)C(=O)N(C)C]

In some cases, R¹ is

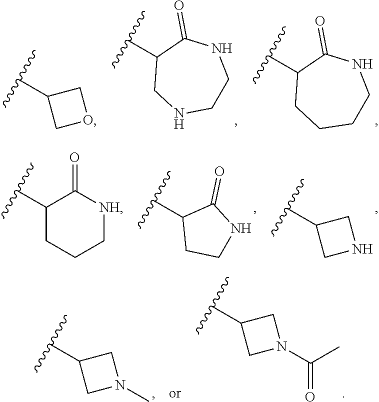

In some cases, R¹ is

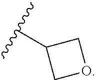 [SMILES: *C1COC1]

In some cases, R¹ is

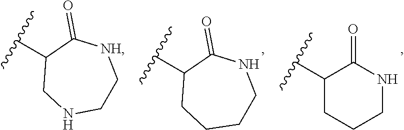

In some cases, R¹ is

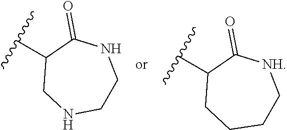 [SMILES: *C1CNCCNC1=O ; *C1CCCCNC1=O]

In some cases, R¹ is

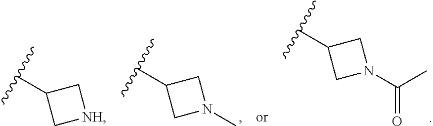

In some cases, R¹ is

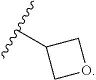 [SMILES: *C1COC1]

In some cases, R¹ is

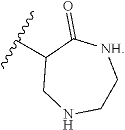 [SMILES: *C1CNCCNC1=O]

In some cases, R¹ is

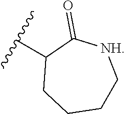 [SMILES: *C1CCCCNC1=O]

In some cases, R¹ is

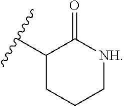 [SMILES: *C1CCCNC1=O]

In some cases, R¹ is

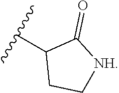 [SMILES: *C1CCNC1=O]

In some cases, R¹ is

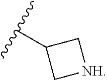 [SMILES: *C1CNC1]

In some cases, R¹ is

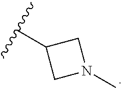 [SMILES: *C1CN(C)C1]

In some cases, R¹ is

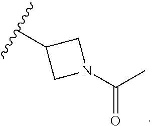 [SMILES: *C1CN(C(C)=O)C1]

In some cases, R¹ is C₂₋₆alkylene —C(O)—NR^{N}R^{N}. In some cases, R¹ is

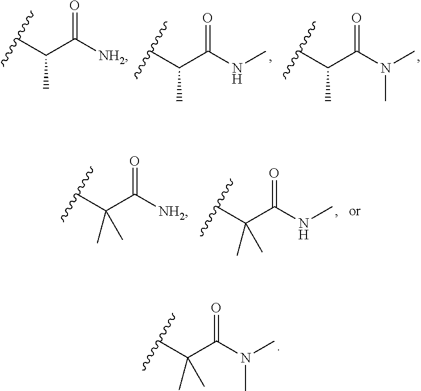

In some cases, R¹ is C₂₋₆hydroxyalkyl. In some cases, R¹ is

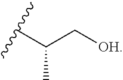 [SMILES: *[C@@H](C)CO]

In some cases, Ar¹ is C₆₋₁₀aryl or 5-10 membered heteroaryl, wherein 1-3 ring atoms are selected from O, N, and S, and Ar¹ is optionally substituted with 1 or 2 R³. In some cases, Ar¹ is C₆aryl or 5-6 membered heteroaryl, and Ar² is optionally substituted with 1 or 2 R³. In some cases, Ar¹ is C₆₋₁₀aryl. In some cases, Ar¹ is phenyl or naphthyl. In some cases, Ar¹ is phenyl. In some cases, Ar¹ is 5-10 membered heteroaryl. In some cases, Ar¹ is 5-6 membered heteroaryl. In some cases, Ar¹ is 5-membered heteroaryl. In some cases, Ar¹ is 6-membered heteroaryl. In some cases, Ar¹ is pyrazolyl, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, or thiadiazolyl, and is optionally substituted with 1 or 2 R³. In some cases, Ar¹ is pyrazolyl, and is optionally substituted with 1 or 2 R³. In some cases, Ar¹ is unsubstituted. In some cases, Ar¹ is substituted with 1 or 2 R³. In some cases, Ar¹ is substituted with 1 R³. In some cases, Ar¹ is substituted with 2 R³. In some cases, at least one R³ is halo. In some cases, each R³ is halo. In some cases, at least one R³ is C₁₋₆alkyl. In some cases, each R³ is C₁₋₆alkyl. In some cases, each R³ independently is fluoro or methyl. In some cases, each R³ is fluoro. In some cases, each R³ is methyl. In some cases, R³ is fluoro, chloro, methyl, ethyl, isopropyl, cyclopropyl, or trifluromethyl. In some cases, Ar¹ is

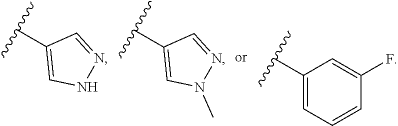

In some cases, Ar¹ is

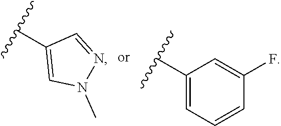

In some cases, Ar¹ is

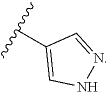 [SMILES: *c1cn[nH]c1]

In some cases, Ar¹ is

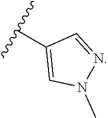 [SMILES: *c1cnn(C)c1]

In some cases, Ar¹ is

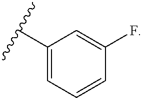 [SMILES: *c1cccc(F)c1]

In some cases, Ar² is C₆₋₁₀aryl or 5-12 membered heteroaryl, wherein 1-3 ring atoms are selected from O, N, and S, and Ar² is optionally substituted with 1, 2, or 3 R⁴. In some cases, Ar² is C₆aryl or 5-6 membered heteroaryl, and Ar² is optionally substituted with 1 or 2 R⁴. In some cases, Ar² is C₆₋₁₀aryl. In some cases, Ar² is phenyl or naphthyl. In some cases, Ar² is phenyl. In some cases, Ar² is 5-12 membered heterocyclyl and is optionally substituted with 1, 2, or 3 R⁴. In some cases, Ar² is 5-12 membered heteroaryl. In some cases, Ar² is 5-6 membered heteroaryl. In some cases, Ar² is 5-membered heteroaryl. In some cases, Ar² is 6-membered heteroaryl. In some cases, Ar² is piperindinyl, piperazinyl, pyrazolyl, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, or thiadiazolyl, and is substituted with 0, 1, or 2 R⁴. In some cases, Ar² is pyrazolyl, and is substituted with 0, 1, or 2 R⁴. In some cases, Ar² is unsubstituted. In some cases, Ar² is substituted with 1, 2, or 3 R⁴ (e.g., 1 or 2 R⁴). In some cases, Ar² is substituted with 1 R⁴. In some cases, Ar² is substituted with 2 R⁴. In some cases, at least one R⁴ is halo. In some cases, at least one R⁴ is OH. In some cases, at least one R⁴ is CN. In some cases, at least one R⁴ is C₁₋₆alkyl. In some cases, at least one R⁴ is methyl, ethyl, propyl, or isopropyl. In some cases, at least one R⁴ is methyl. In some cases, at least one R⁴ is C₁₋₆haloalkyl. In some cases, at least one R⁴ is CF₃ or CHF₂, or CH₂F. In some cases, at least one R⁴ is C₁₋₆ hydroxyalkyl. In some cases, at least one R⁴ is C₁₋₆alkoxy. In some cases, at least one R⁴ is OCH₃. In some cases, at least one R⁴ is C₁₋₆haloalkoxy. In some cases, at least one R⁴ is OCF₃ or OCHF₂. In some cases, at least one R⁴ is NR^{N}R^{N}. In some cases, at least one R⁴ is NH₂, NHCH₃, or N(CH₃)₂. In some cases, at least one R⁴ is NH₂ or N(CH₃)₂. In some cases, at least one R⁴ is CONR^{N}R^{N}. In some cases, at least one R⁴ is CON(CH₃)₂, CONHCH₃, or CONH₂. In some cases, at least one R⁴ is N(CH₃)₂, NHCH₃, or CONH₂. In some cases, at least one R⁴ is COOH. In some cases, at least one R⁴ is COO—C₁₋₆alkyl. In some cases, at least one R⁴ is C(O)—C₁₋₆alkyl. In some cases, at least one R⁴ is COCH₃. In some cases, at least one R⁴ is SO₂—C₁₋₆alkyl. In some cases, at least one R⁴ is SO₂CH₃. In some cases, at least one R⁴ is C₃₋₆cycloalkyl. In some cases, at least one R⁴ comprises cyclopropyl, cyclobutyl, cyclopentyl, or cyclohexyl. In some cases, at least one R⁴ comprises cyclopropyl. In some cases, at least one R⁴ is 4-12 membered heterocyclyl. In some cases, at least one R⁴ comprises morpholino. In some cases, at least one R⁴ is fluoro, chloro, OH, CN, CH₃, OCH₃, CF₃, OCF₃, CHF₂, CH₂F, OCHF₂, NH₂, N(CH₃)₂, CONH₂, COOH, SO₂CH₃, cyclopropyl, or morpholino. In some cases, at least one R⁴ is fluoro or chloro. In some cases, at least one R⁴ is fluoro. In some cases, at least one R⁴ is chloro. In some cases, each R⁴ is halo. In some cases, each R⁴ independently is Cl or F. In some cases, each R⁴ is OH. In some cases, each R⁴ is CN. In some cases, each R⁴ is C₁₋₆haloalkyl. In some cases, each R⁴ is C₁₋₆hydroxyalkyl. In some cases, each R⁴ is C₁₋₆alkoxy. In some cases, each R⁴ is C₁₋₆haloalkoxy. In some cases, each R⁴ is NR^{N}R^{N}. In some cases, each R⁴ is CONR^{N}R^{N}. In some cases, each R⁴ is COOH. In some cases, each R⁴ is COO—C₁₋₆alkyl. In some cases, each R⁴ is C(O)—C₁₋₆alkyl. In some cases, each R⁴ is SO₂—C₁₋₆alkyl. In some cases, each R⁴ is C₃₋₈cycloalkyl. In some cases, each R⁴ is 4-12 membered heterocyclyl. In some cases, at least one R⁴ selected from CONR^{N}R^{N}, COOH, COO—C₁₋₆alkyl, C(O)—C₁₋₆alkyl, SO₂—C₁₋₆alkyl, C₃₋₈cycloalkyl, 4-12 membered heterocyclyl, 5-12 membered heteroaryl, and phenyl, and the phenyl, heterocyclyl or heteroaryl is substituted with 0, 1, or 2 substituents independently selected from halo and C₁₋₆alkyl. In some cases, Ar² is phenyl, pyridyl, pyrimidinyl, pyrazolyl, or triazolyl, and at least one R⁴ is phenyl or halo substituted-phenyl.

In cases where Ar² is substituted by 1, 2, or 3 R⁴, the R⁴ can be at any position on the ring. In some cases, Ar² is phenyl substituted by 1 or 2 R⁴ at any position on the ring. In some cases, Ar² is

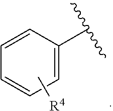

In some cases, Ar² is

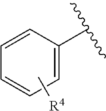

and R⁴ is fluoro, chloro, OH, CN, CH₂, CH₂CH, CH(CH₃)₂, C (CH₃)₃, OCH₃, CF₃, OCF₃, CHF₂, CH₂F, OCHF₂, NH₂, N(CH₃)₂, CONH₂, COOH, SO₂CH₃, cyclopropyl, or morpholino. In some cases, Ar² is

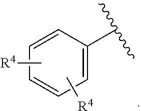

In some cases, Ar² is

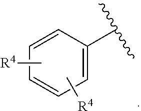

and each R⁴ is independently fluoro, chloro, OH, CN, CH₃, CH₂CH₃, CH(CH₃)₂, C(CH₃)₃, OCH₃, CF₃, OCF₃, CHF₂, CH₂F, OCHF₂, NH₂, N(CH₃)₂, CONH₂, COOH, SO₂CH₃, cyclopropyl, or morpholino. In some cases, Ar² is

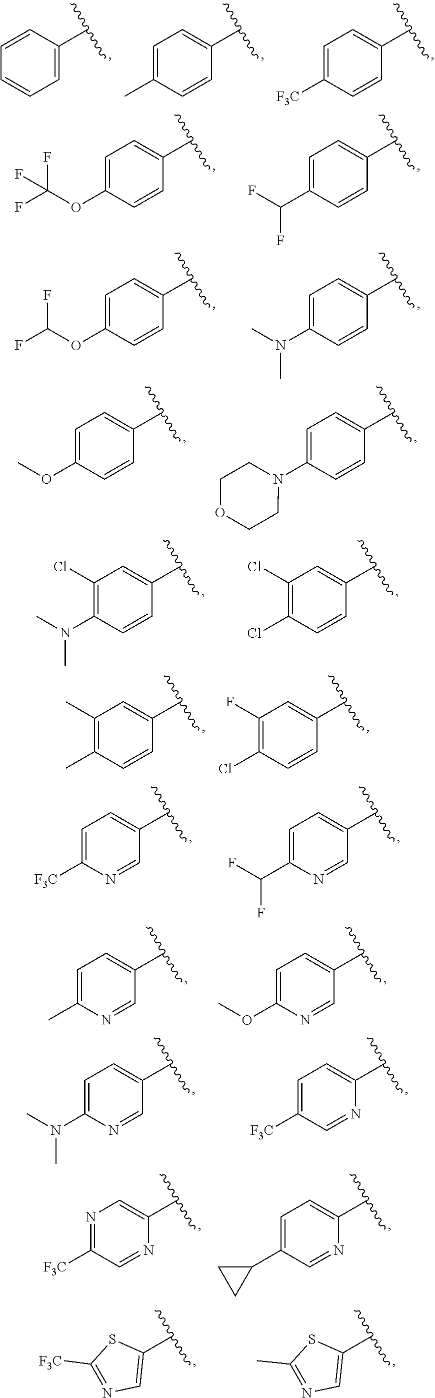

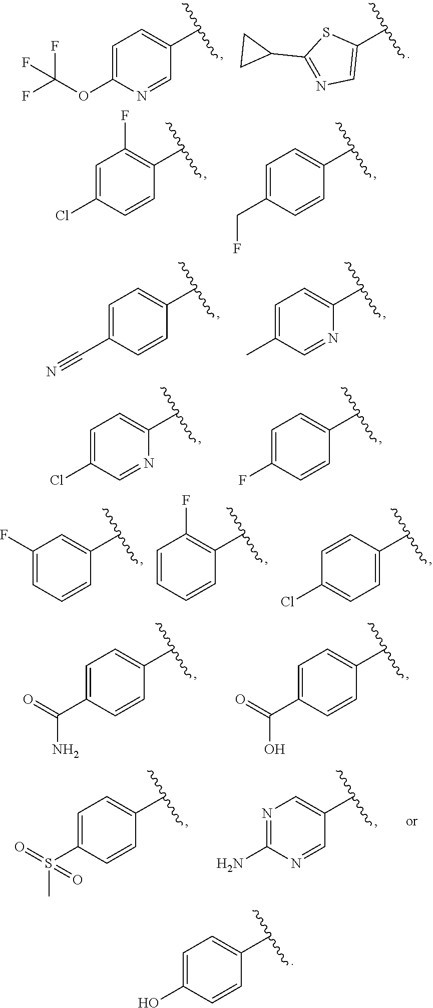

In some cases, Ar² is

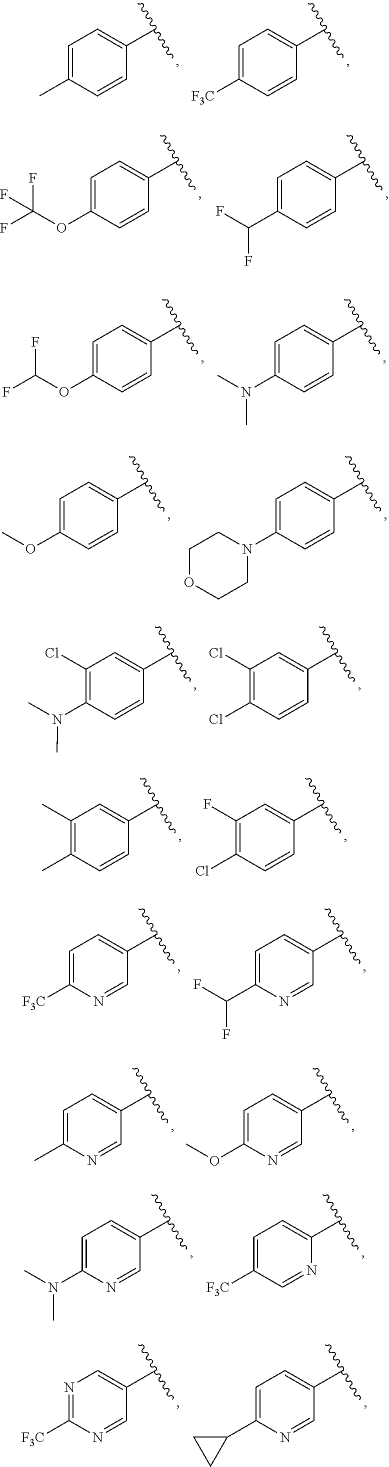

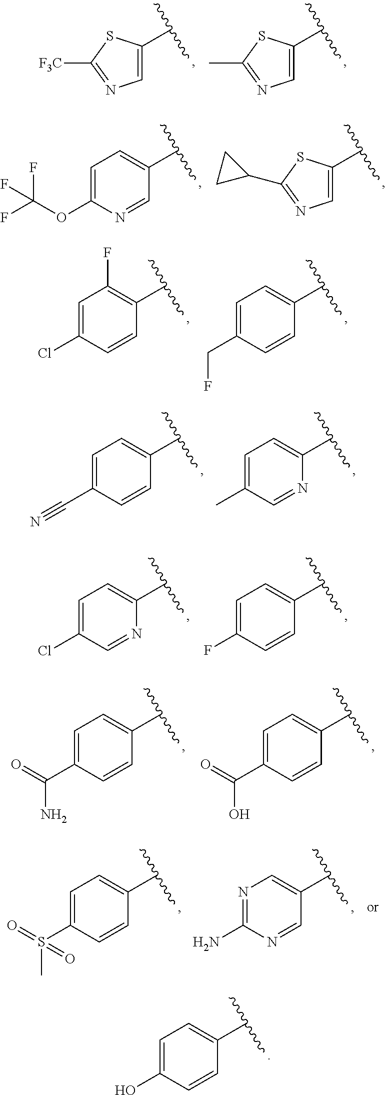

In some cases, Ar² is

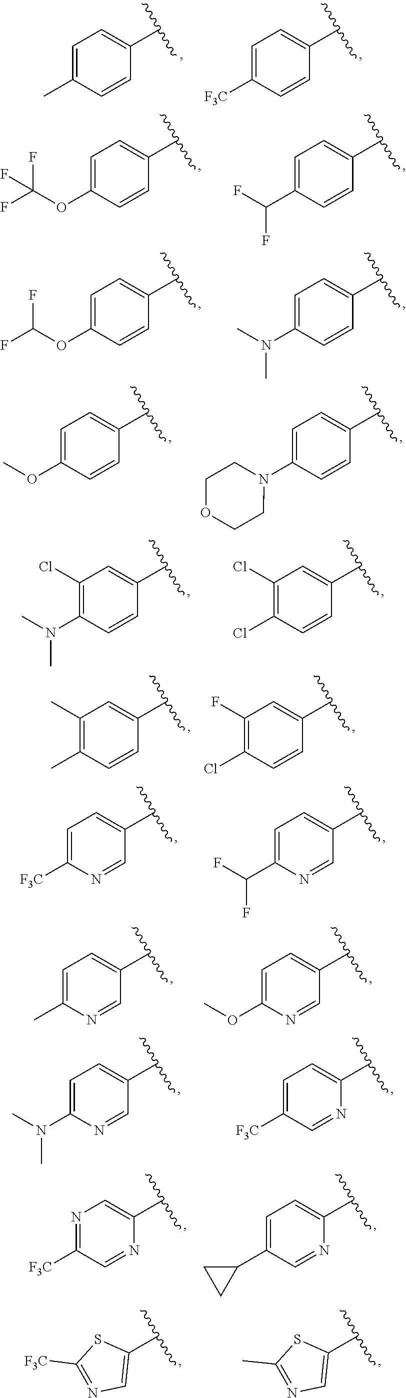

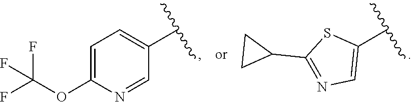 [SMILES: *c1ccc(OC(F)(F)F)nc1 ; *c1cnc(C2CC2)s1]

In some cases, Ar² is

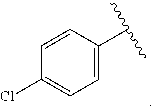 [SMILES: *c1ccc(Cl)cc1]

In some cases Ar² is

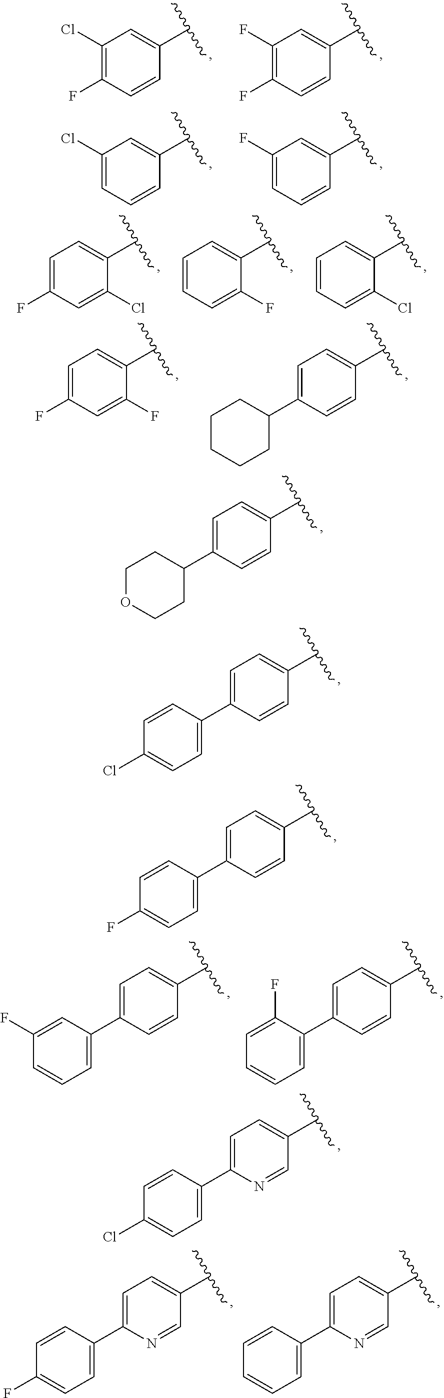

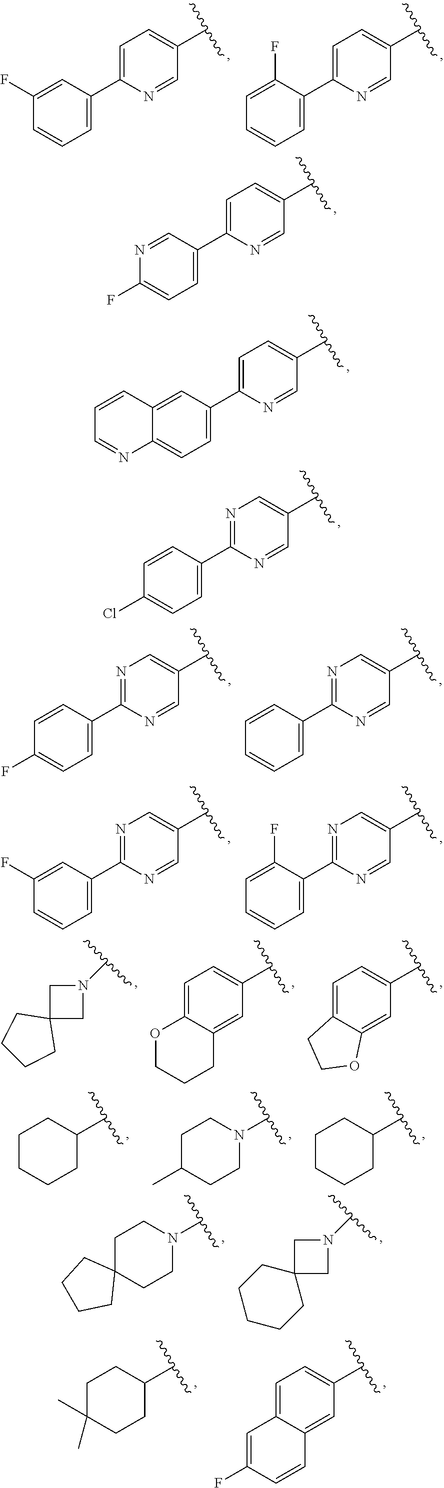

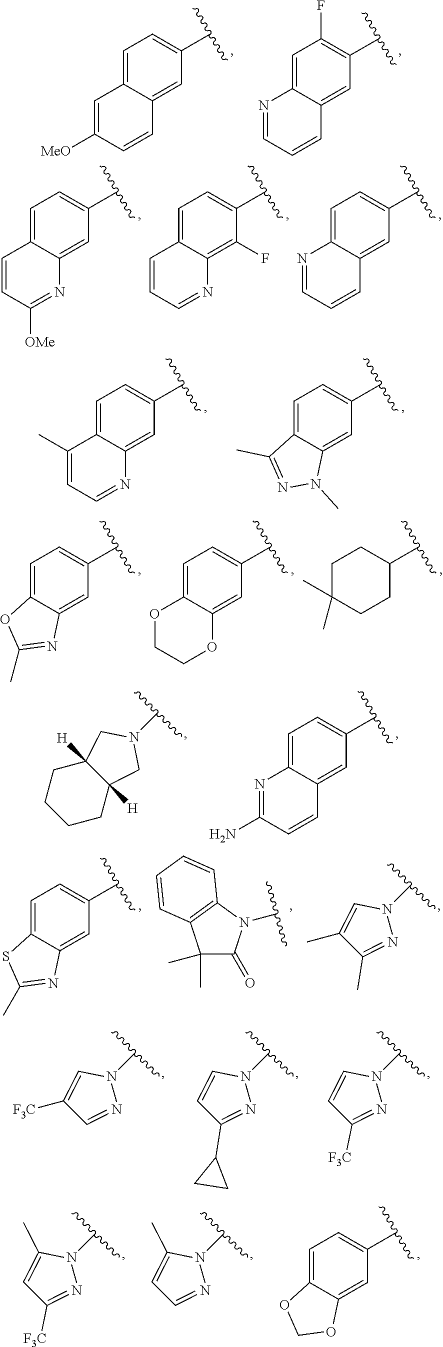

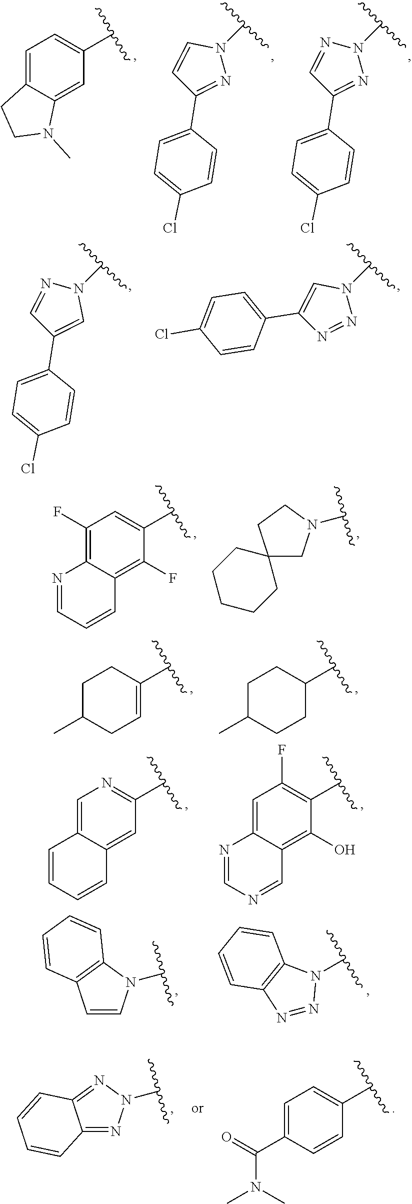

Contemplated compounds of Formula (I) include, but are not limited to the compounds listed in Table 1.

TABLE 1

| # | Structure |
|---|---|
| 1 | 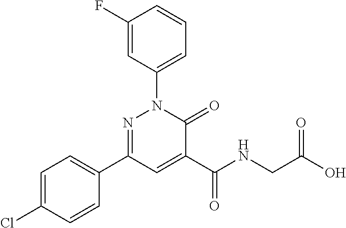 [SMILES: O=C(O)CNC(=O)c1cc(-c2ccc(Cl)cc2)nn(-c2cccc(F)c2)c1=O] |
| 2 | 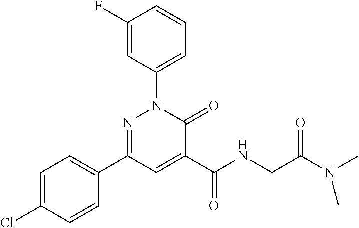 [SMILES: CN(C)C(=O)CNC(=O)c1cc(-c2ccc(Cl)cc2)nn(-c2cccc(F)c2)c1=O] |
| 3 | 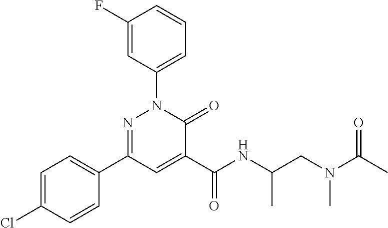 [SMILES: CC(=O)N(C)CC(C)NC(=O)c1cc(-c2ccc(Cl)cc2)nn(-c2cccc(F)c2)c1=O] |
| 4 | 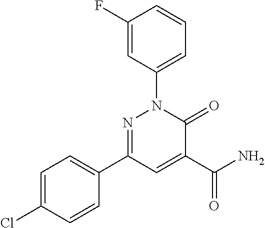 [SMILES: NC(=O)c1cc(-c2ccc(Cl)cc2)nn(-c2cccc(F)c2)c1=O] |
| 5 | 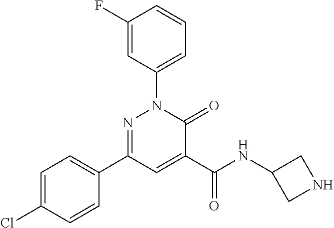 [SMILES: O=C(NC1CNC1)c1cc(-c2ccc(Cl)cc2)nn(-c2cccc(F)c2)c1=O] |
| 6 | 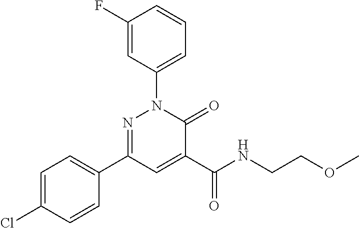 [SMILES: COCCNC(=O)c1cc(-c2ccc(Cl)cc2)nn(-c2cccc(F)c2)c1=O] |
| 7 | 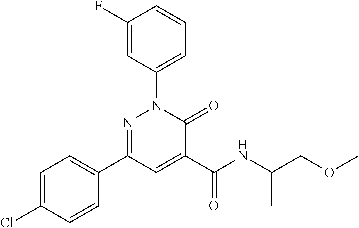 [SMILES: COCC(C)NC(=O)c1cc(-c2ccc(Cl)cc2)nn(-c2cccc(F)c2)c1=O] |
| 8 | 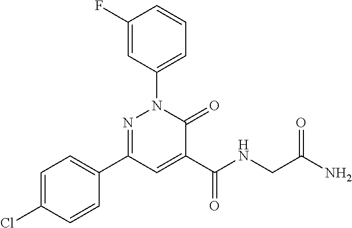 [SMILES: NC(=O)CNC(=O)c1cc(-c2ccc(Cl)cc2)nn(-c2cccc(F)c2)c1=O] |
| 9 | 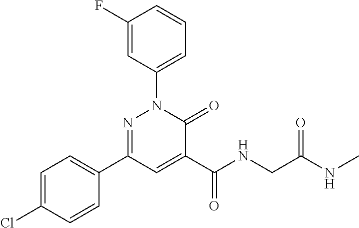 [SMILES: CNC(=O)CNC(=O)c1cc(-c2ccc(Cl)cc2)nn(-c2cccc(F)c2)c1=O] |
| 10 | 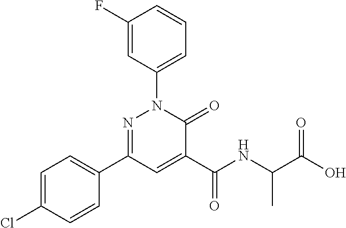 [SMILES: CC(NC(=O)c1cc(-c2ccc(Cl)cc2)nn(-c2cccc(F)c2)c1=O)C(=O)O] |
| 11 | 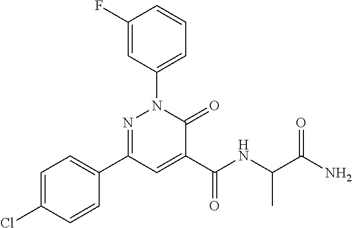 [SMILES: CC(NC(=O)c1cc(-c2ccc(Cl)cc2)nn(-c2cccc(F)c2)c1=O)C(N)=O] |
| 12 | 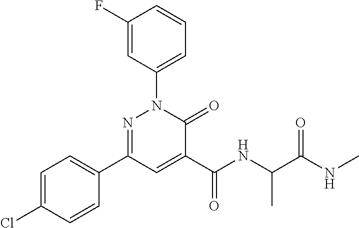 [SMILES: CNC(=O)C(C)NC(=O)c1cc(-c2ccc(Cl)cc2)nn(-c2cccc(F)c2)c1=O] |
| 13 | 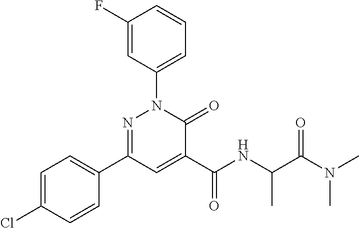 [SMILES: CC(NC(=O)c1cc(-c2ccc(Cl)cc2)nn(-c2cccc(F)c2)c1=O)C(=O)N(C)C] |
| 14 | 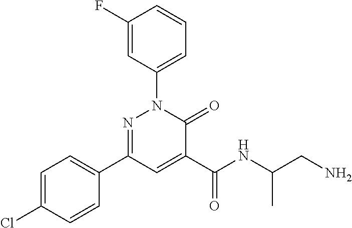 [SMILES: CC(CN)NC(=O)c1cc(-c2ccc(Cl)cc2)nn(-c2cccc(F)c2)c1=O] |
| 15 | 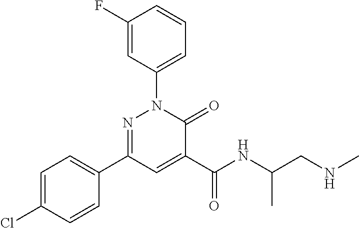 [SMILES: CNCC(C)NC(=O)c1cc(-c2ccc(Cl)cc2)nn(-c2cccc(F)c2)c1=O] |
| 16 | 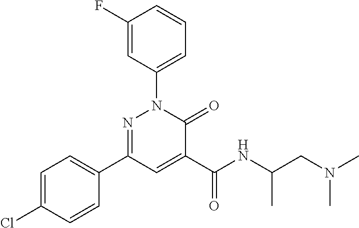 [SMILES: CC(CN(C)C)NC(=O)c1cc(-c2ccc(Cl)cc2)nn(-c2cccc(F)c2)c1=O] |
| 17 | 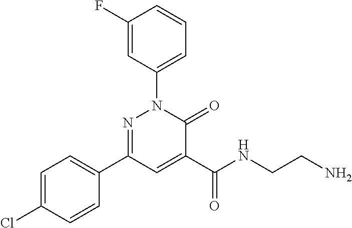 [SMILES: NCCNC(=O)c1cc(-c2ccc(Cl)cc2)nn(-c2cccc(F)c2)c1=O] |
| 18 | 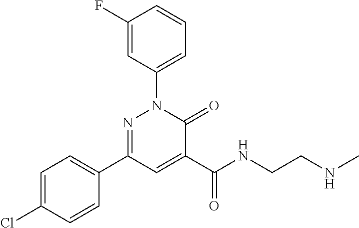 [SMILES: CNCCNC(=O)c1cc(-c2ccc(Cl)cc2)nn(-c2cccc(F)c2)c1=O] |
| 19 | 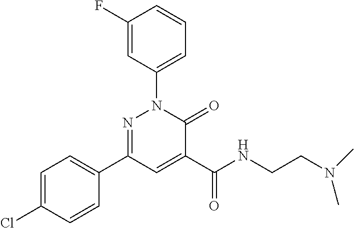 [SMILES: CN(C)CCNC(=O)c1cc(-c2ccc(Cl)cc2)nn(-c2cccc(F)c2)c1=O] |
| 20 | 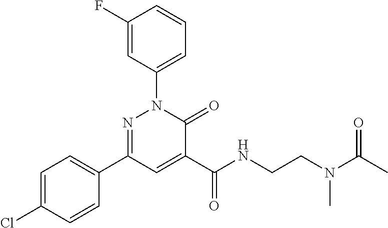 [SMILES: CC(=O)N(C)CCNC(=O)c1cc(-c2ccc(Cl)cc2)nn(-c2cccc(F)c2)c1=O] |
| 21 | 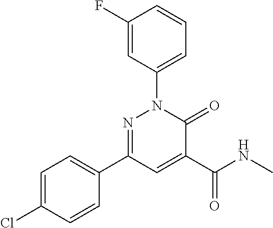 [SMILES: CNC(=O)c1cc(-c2ccc(Cl)cc2)nn(-c2cccc(F)c2)c1=O] |
| 22 | 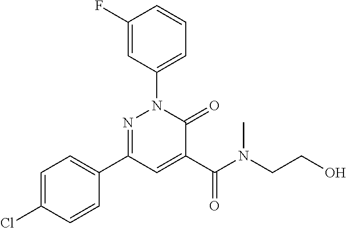 [SMILES: CN(CCO)C(=O)c1cc(-c2ccc(Cl)cc2)nn(-c2cccc(F)c2)c1=O] |
| 23 | 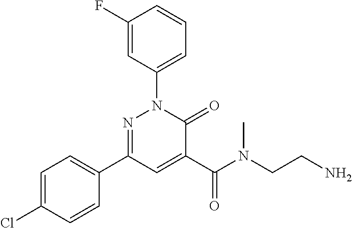 [SMILES: CN(CCN)C(=O)c1cc(-c2ccc(Cl)cc2)nn(-c2cccc(F)c2)c1=O] |
| 24 | 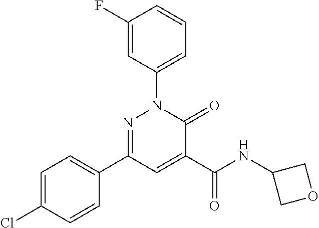 [SMILES: O=C(NC1COC1)c1cc(-c2ccc(Cl)cc2)nn(-c2cccc(F)c2)c1=O] |
| 25 | 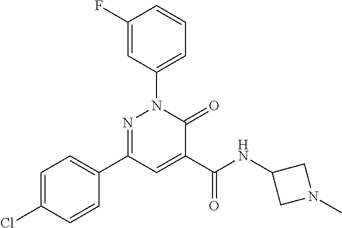 [SMILES: CN1CC(NC(=O)c2cc(-c3ccc(Cl)cc3)nn(-c3cccc(F)c3)c2=O)C1] |
| 26 | 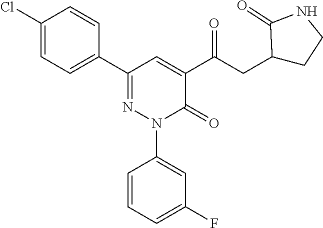 [SMILES: O=C(CC1CCNC1=O)c1cc(-c2ccc(Cl)cc2)nn(-c2cccc(F)c2)c1=O] |
| 27 | 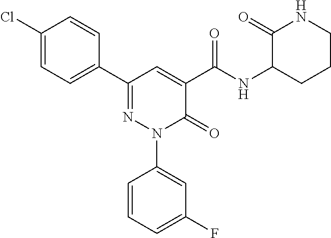 [SMILES: O=C(NC1CCCNC1=O)c1cc(-c2ccc(Cl)cc2)nn(-c2cccc(F)c2)c1=O] |
| 28 | 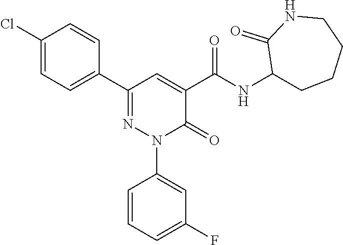 [SMILES: O=C(NC1CCCCNC1=O)c1cc(-c2ccc(Cl)cc2)nn(-c2cccc(F)c2)c1=O] |
| 28- stereo pure | 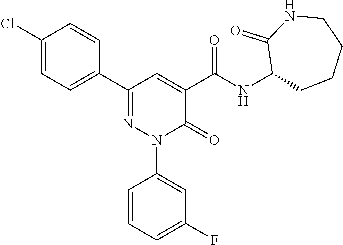 [SMILES: O=C(N[C@H]1CCCCNC1=O)c1cc(-c2ccc(Cl)cc2)nn(-c2cccc(F)c2)c1=O] |
| 29 | 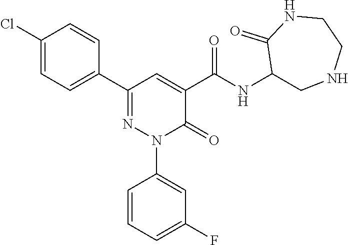 [SMILES: O=C(NC1CNCCNC1=O)c1cc(-c2ccc(Cl)cc2)nn(-c2cccc(F)c2)c1=O] |
| 30 | 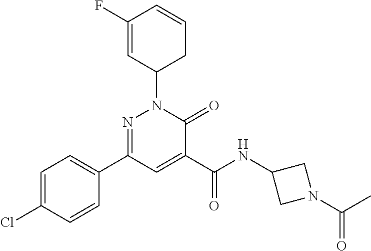 [SMILES: CC(=O)N1CC(NC(=O)c2cc(-c3ccc(Cl)cc3)nn(C3C=C(F)C=CC3)c2=O)C1] |
| 31 | 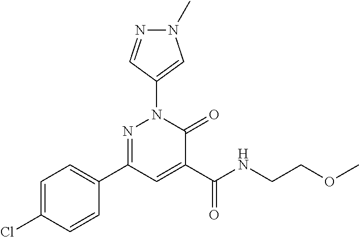 [SMILES: COCCNC(=O)c1cc(-c2ccc(Cl)cc2)nn(-c2cnn(C)c2)c1=O] |
| 32 | 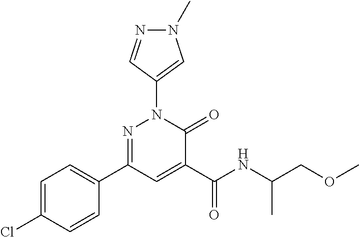 [SMILES: COCC(C)NC(=O)c1cc(-c2ccc(Cl)cc2)nn(-c2cnn(C)c2)c1=O] |
| 33 | 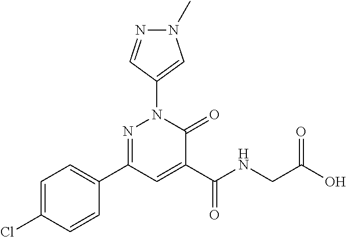 [SMILES: Cn1cc(-n2nc(-c3ccc(Cl)cc3)cc(C(=O)NCC(=O)O)c2=O)cn1] |
| 34 | 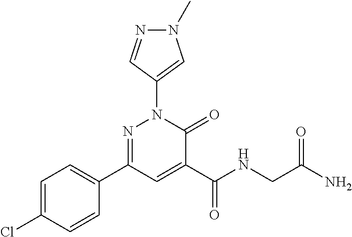 [SMILES: Cn1cc(-n2nc(-c3ccc(Cl)cc3)cc(C(=O)NCC(N)=O)c2=O)cn1] |
| 35 | 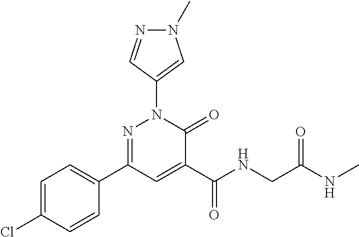 [SMILES: CNC(=O)CNC(=O)c1cc(-c2ccc(Cl)cc2)nn(-c2cnn(C)c2)c1=O] |
| 36 | 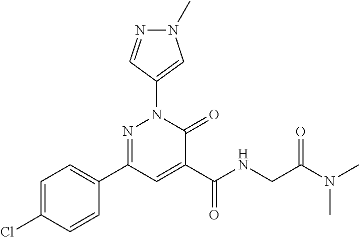 [SMILES: CN(C)C(=O)CNC(=O)c1cc(-c2ccc(Cl)cc2)nn(-c2cnn(C)c2)c1=O] |
| 37 | 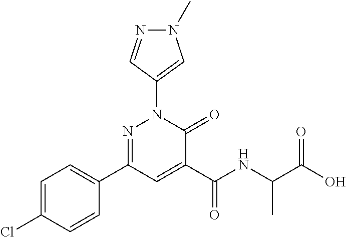 [SMILES: CC(NC(=O)c1cc(-c2ccc(Cl)cc2)nn(-c2cnn(C)c2)c1=O)C(=O)O] |
| 38 | 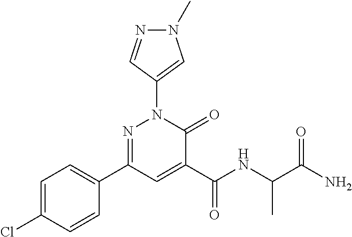 [SMILES: CC(NC(=O)c1cc(-c2ccc(Cl)cc2)nn(-c2cnn(C)c2)c1=O)C(N)=O] |
| 39 | 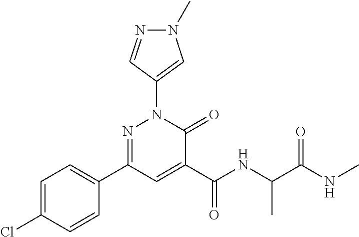 [SMILES: CNC(=O)C(C)NC(=O)c1cc(-c2ccc(Cl)cc2)nn(-c2cnn(C)c2)c1=O] |
| 40 | 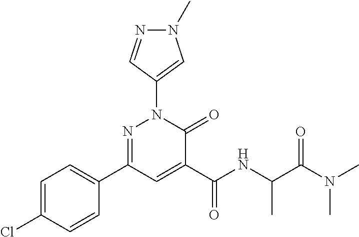 [SMILES: CC(NC(=O)c1cc(-c2ccc(Cl)cc2)nn(-c2cnn(C)c2)c1=O)C(=O)N(C)C] |
| 41 | 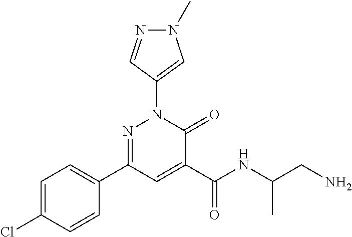 [SMILES: CC(CN)NC(=O)c1cc(-c2ccc(Cl)cc2)nn(-c2cnn(C)c2)c1=O] |
| 42 | 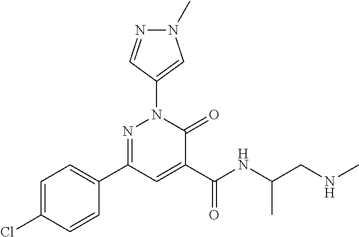 [SMILES: CNCC(C)NC(=O)c1cc(-c2ccc(Cl)cc2)nn(-c2cnn(C)c2)c1=O] |
| 43 | 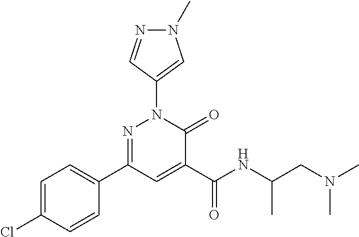 [SMILES: CC(CN(C)C)NC(=O)c1cc(-c2ccc(Cl)cc2)nn(-c2cnn(C)c2)c1=O] |
| 44 | 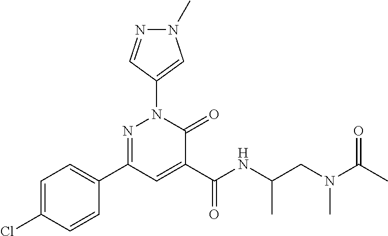 [SMILES: CC(=O)N(C)CC(C)NC(=O)c1cc(-c2ccc(Cl)cc2)nn(-c2cnn(C)c2)c1=O] |
| 45 | 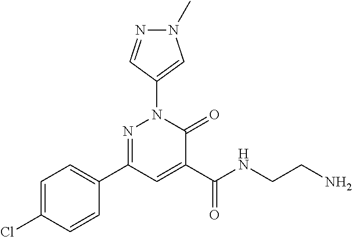 [SMILES: Cn1cc(-n2nc(-c3ccc(Cl)cc3)cc(C(=O)NCCN)c2=O)cn1] |
| 46 | 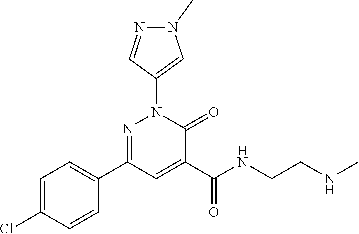 [SMILES: CNCCNC(=O)c1cc(-c2ccc(Cl)cc2)nn(-c2cnn(C)c2)c1=O] |
| 47 | 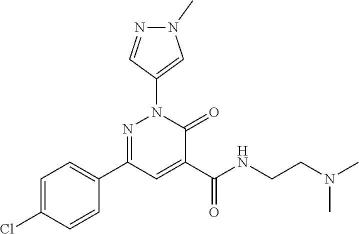 [SMILES: CN(C)CCNC(=O)c1cc(-c2ccc(Cl)cc2)nn(-c2cnn(C)c2)c1=O] |
| 48 | 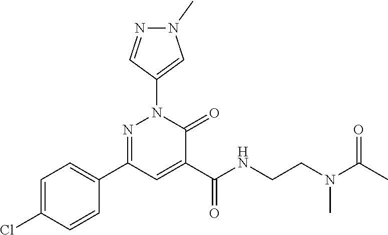 [SMILES: CC(=O)N(C)CCNC(=O)c1cc(-c2ccc(Cl)cc2)nn(-c2cnn(C)c2)c1=O] |
| 49 | 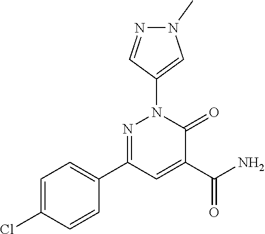 [SMILES: Cn1cc(-n2nc(-c3ccc(Cl)cc3)cc(C(N)=O)c2=O)cn1] |
| 50 | 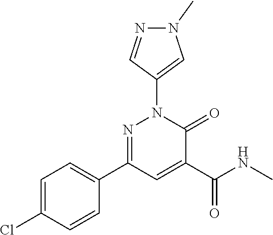 [SMILES: CNC(=O)c1cc(-c2ccc(Cl)cc2)nn(-c2cnn(C)c2)c1=O] |
| 51 | 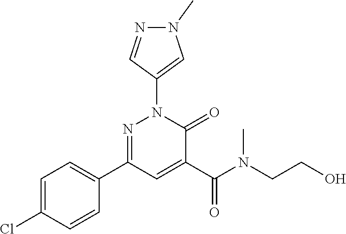 [SMILES: CN(CCO)C(=O)c1cc(-c2ccc(Cl)cc2)nn(-c2cnn(C)c2)c1=O] |
| 52 | 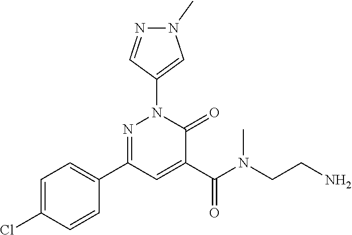 [SMILES: CN(CCN)C(=O)c1cc(-c2ccc(Cl)cc2)nn(-c2cnn(C)c2)c1=O] |
| 53 | 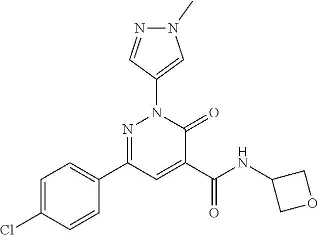 [SMILES: Cn1cc(-n2nc(-c3ccc(Cl)cc3)cc(C(=O)NC3COC3)c2=O)cn1] |
| 54 | 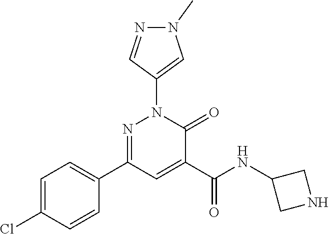 [SMILES: Cn1cc(-n2nc(-c3ccc(Cl)cc3)cc(C(=O)NC3CNC3)c2=O)cn1] |
| 55 | 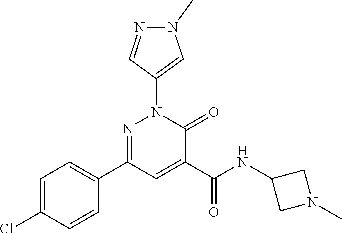 [SMILES: CN1CC(NC(=O)c2cc(-c3ccc(Cl)cc3)nn(-c3cnn(C)c3)c2=O)C1] |
| 56 | 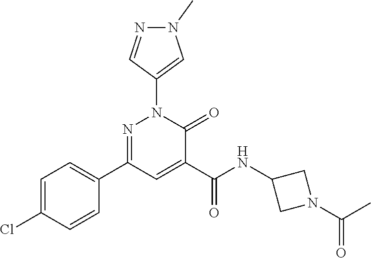 [SMILES: CC(=O)N1CC(NC(=O)c2cc(-c3ccc(Cl)cc3)nn(-c3cnn(C)c3)c2=O)C1] |
| 57 | 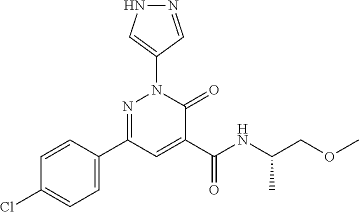 [SMILES: COC[C@H](C)NC(=O)c1cc(-c2ccc(Cl)cc2)nn(-c2cn[nH]c2)c1=O] |
| 58 | 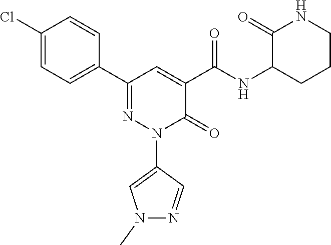 [SMILES: Cn1cc(-n2nc(-c3ccc(Cl)cc3)cc(C(=O)NC3CCCNC3=O)c2=O)cn1] |
| 59 | 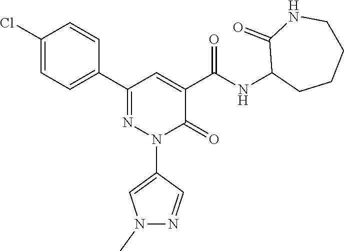 [SMILES: Cn1cc(-n2nc(-c3ccc(Cl)cc3)cc(C(=O)NC3CCCCNC3=O)c2=O)cn1] |
| 59- stereo pure | 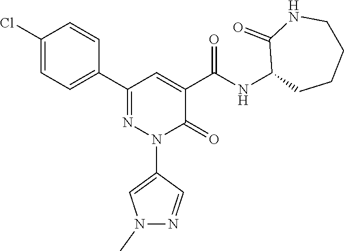 [SMILES: Cn1cc(-n2nc(-c3ccc(Cl)cc3)cc(C(=O)N[C@H]3CCCCNC3=O)c2=O)cn1] |
| 60 | 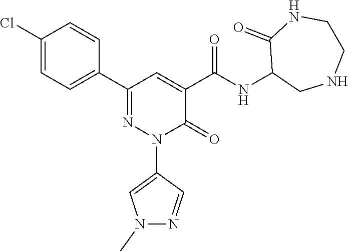 [SMILES: Cn1cc(-n2nc(-c3ccc(Cl)cc3)cc(C(=O)NC3CNCCNC3=O)c2=O)cn1] |
| 61 | 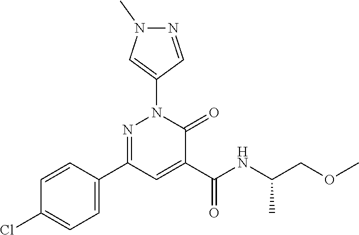 [SMILES: COC[C@H](C)NC(=O)c1cc(-c2ccc(Cl)cc2)nn(-c2cnn(C)c2)c1=O] |
| 62 | 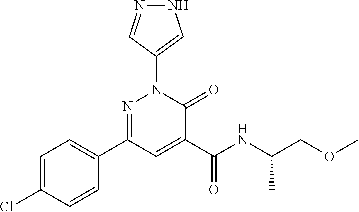 [SMILES: COC[C@H](C)NC(=O)c1cc(-c2ccc(Cl)cc2)nn(-c2cn[nH]c2)c1=O] |
| 63 | 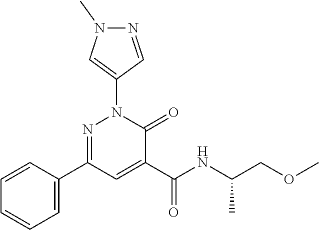 [SMILES: COC[C@H](C)NC(=O)c1cc(-c2ccccc2)nn(-c2cnn(C)c2)c1=O] |
| 64 | 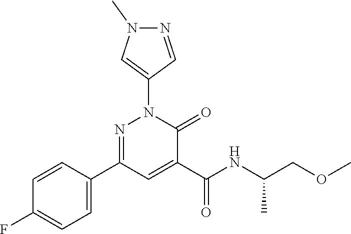 [SMILES: COC[C@H](C)NC(=O)c1cc(-c2ccc(F)cc2)nn(-c2cnn(C)c2)c1=O] |
| 65 | 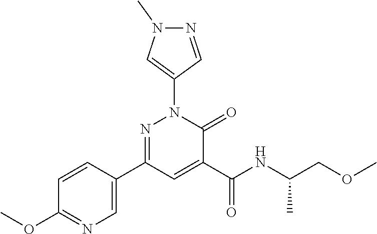 |
| 66 | 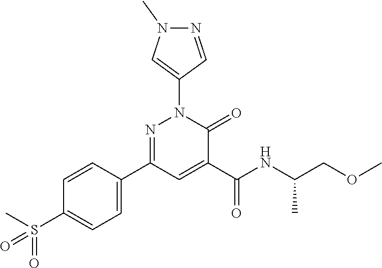 [SMILES: COC[C@H](C)NC(=O)c1cc(-c2ccc(S(C)(=O)=O)cc2)nn(-c2cnn(C)c2)c1=O] |
| 67 | 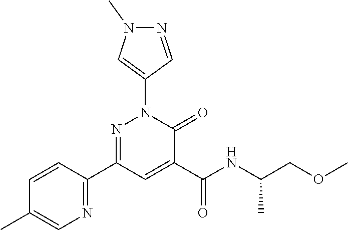 |
| 68 | 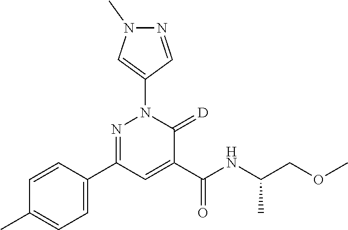 |
| 69 | 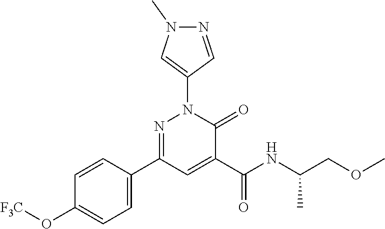 [SMILES: COC[C@H](C)NC(=O)c1cc(-c2ccc(OC(F)(F)F)cc2)nn(-c2cnn(C)c2)c1=O] |
| 70 | 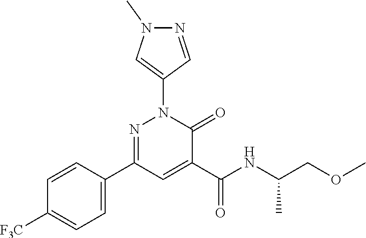 [SMILES: COC[C@H](C)NC(=O)c1cc(-c2ccc(C(F)(F)F)cc2)nn(-c2cnn(C)c2)c1=O] |
| 71 | 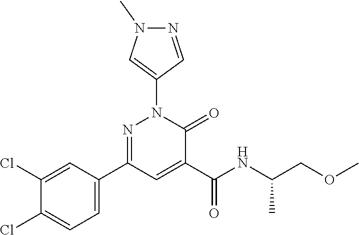 [SMILES: COC[C@H](C)NC(=O)c1cc(-c2ccc(Cl)c(Cl)c2)nn(-c2cnn(C)c2)c1=O] |
| 72 | 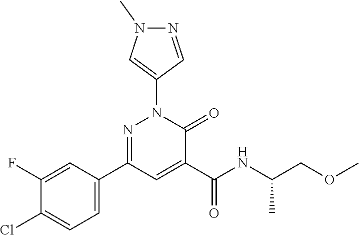 [SMILES: COC[C@H](C)NC(=O)c1cc(-c2ccc(Cl)c(F)c2)nn(-c2cnn(C)c2)c1=O] |
| 73 | 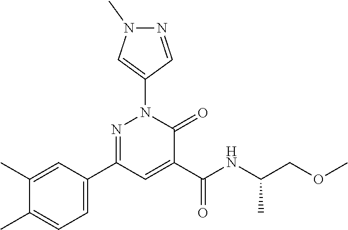 |
| 74 | 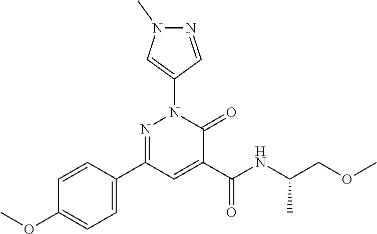 |
| 75 | 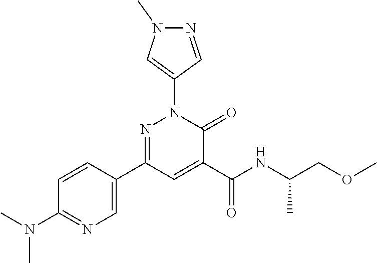 |
| 76 | 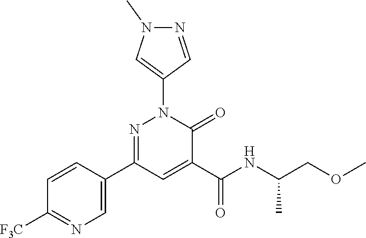 [SMILES: COC[C@H](C)NC(=O)c1cc(-c2ccc(C(F)(F)F)nc2)nn(-c2cnn(C)c2)c1=O] |
| 77 | 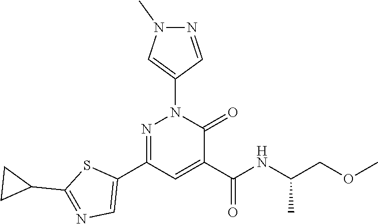 [SMILES: COC[C@H](C)NC(=O)c1cc(-c2cnc(C3CC3)s2)nn(-c2cnn(C)c2)c1=O] |
| 78 | 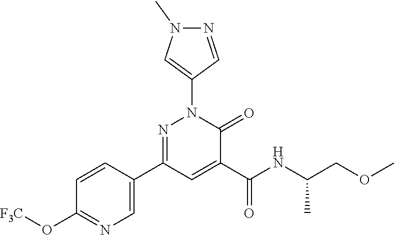 [SMILES: COC[C@H](C)NC(=O)c1cc(-c2ccc(OC(F)(F)F)nc2)nn(-c2cnn(C)c2)c1=O] |
| 79 | 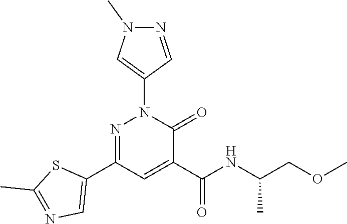 |
| 80 | 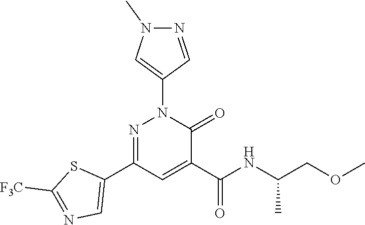 [SMILES: COC[C@H](C)NC(=O)c1cc(-c2cnc(C(F)(F)F)s2)nn(-c2cnn(C)c2)c1=O] |
| 81 | 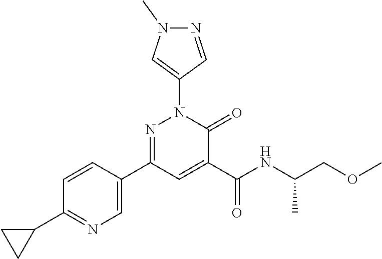 [SMILES: COC[C@H](C)NC(=O)c1cc(-c2ccc(C3CC3)nc2)nn(-c2cnn(C)c2)c1=O] |
| 82 | 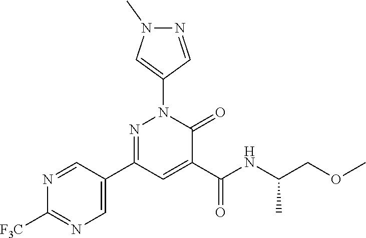 [SMILES: COC[C@H](C)NC(=O)c1cc(-c2cnc(C(F)(F)F)nc2)nn(-c2cnn(C)c2)c1=O] |
| 83 | 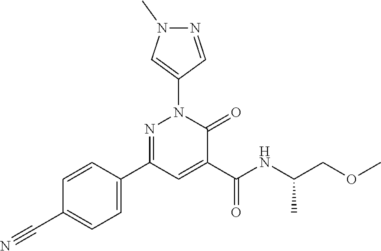 [SMILES: COC[C@H](C)NC(=O)c1cc(-c2ccc(C#N)cc2)nn(-c2cnn(C)c2)c1=O] |
| 84 | 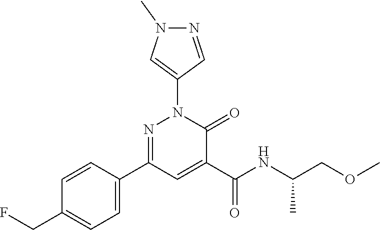 [SMILES: COC[C@H](C)NC(=O)c1cc(-c2ccc(CF)cc2)nn(-c2cnn(C)c2)c1=O] |
| 85 | 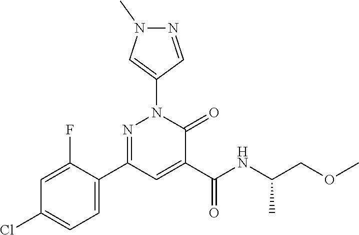 [SMILES: COC[C@H](C)NC(=O)c1cc(-c2ccc(Cl)cc2F)nn(-c2cnn(C)c2)c1=O] |
| 86 | 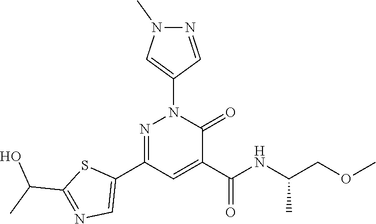 [SMILES: COC[C@H](C)NC(=O)c1cc(-c2cnc(C(C)O)s2)nn(-c2cnn(C)c2)c1=O] |
| 87 | 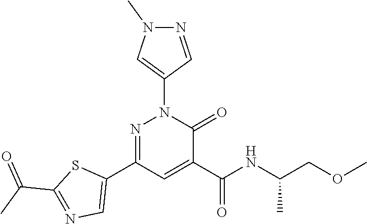 [SMILES: COC[C@H](C)NC(=O)c1cc(-c2cnc(C(C)=O)s2)nn(-c2cnn(C)c2)c1=O] |
| 88 | 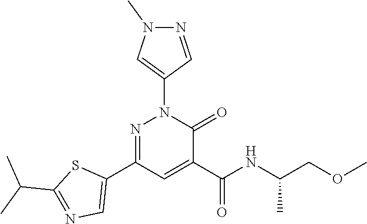 [SMILES: COC[C@H](C)NC(=O)c1cc(-c2cnc(C(C)C)s2)nn(-c2cnn(C)c2)c1=O] |
| 89 | 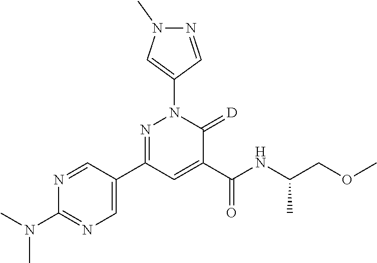 |
| 90 | 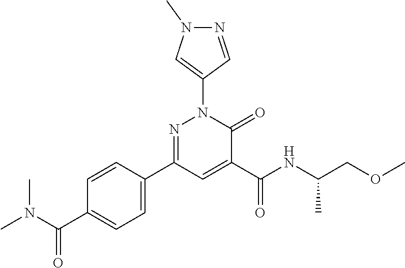 |
| 91 | 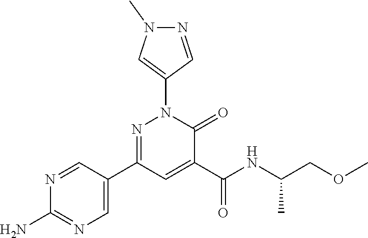 [SMILES: COC[C@H](C)NC(=O)c1cc(-c2cnc(N)nc2)nn(-c2cnn(C)c2)c1=O] |
| 92 | 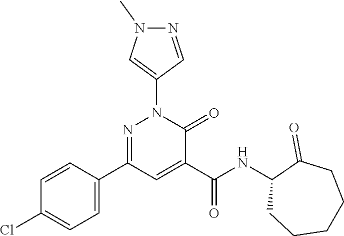 [SMILES: Cn1cc(-n2nc(-c3ccc(Cl)cc3)cc(C(=O)N[C@H]3CCCCCC3=O)c2=O)cn1] |
| 93 | 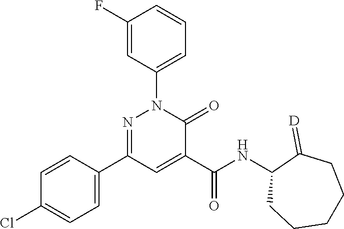 |
| 94 | 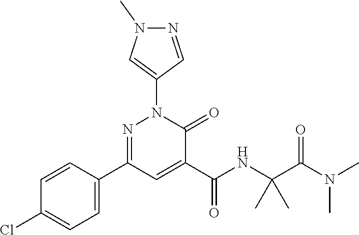 [SMILES: CN(C)C(=O)C(C)(C)NC(=O)c1cc(-c2ccc(Cl)cc2)nn(-c2cnn(C)c2)c1=O] |
| 95 | 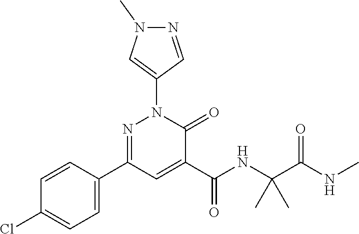 [SMILES: CNC(=O)C(C)(C)NC(=O)c1cc(-c2ccc(Cl)cc2)nn(-c2cnn(C)c2)c1=O] |
| 96 | 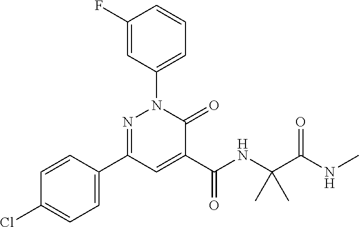 [SMILES: CNC(=O)C(C)(C)NC(=O)c1cc(-c2ccc(Cl)cc2)nn(-c2cccc(F)c2)c1=O] |
| 97 | 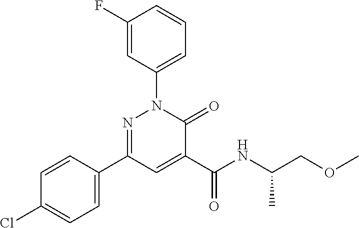 [SMILES: COC[C@H](C)NC(=O)c1cc(-c2ccc(Cl)cc2)nn(-c2cccc(F)c2)c1=O] |
| 98 | 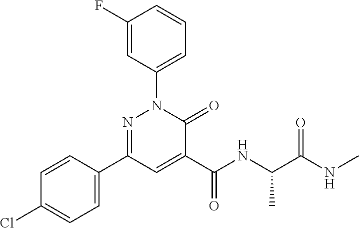 [SMILES: CNC(=O)[C@H](C)NC(=O)c1cc(-c2ccc(Cl)cc2)nn(-c2cccc(F)c2)c1=O] |
| 99 | 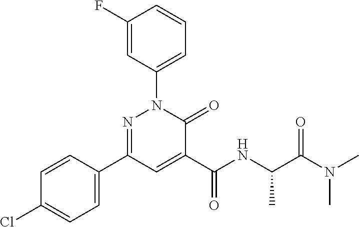 [SMILES: C[C@H](NC(=O)c1cc(-c2ccc(Cl)cc2)nn(-c2cccc(F)c2)c1=O)C(=O)N(C)C] |
| 100 | 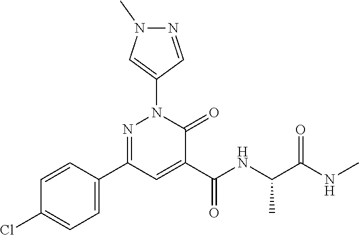 [SMILES: CNC(=O)[C@H](C)NC(=O)c1cc(-c2ccc(Cl)cc2)nn(-c2cnn(C)c2)c1=O] |
| 101 | 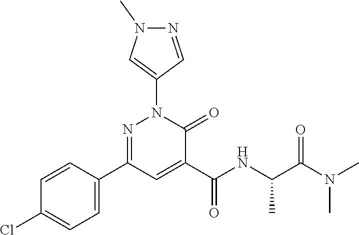 [SMILES: C[C@H](NC(=O)c1cc(-c2ccc(Cl)cc2)nn(-c2cnn(C)c2)c1=O)C(=O)N(C)C] |
| 102 | 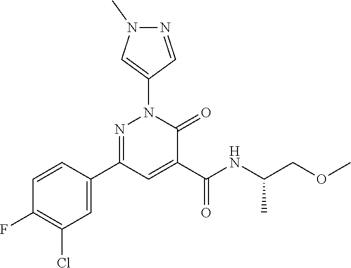 [SMILES: COC[C@H](C)NC(=O)c1cc(-c2ccc(F)c(Cl)c2)nn(-c2cnn(C)c2)c1=O] |
| 103 | 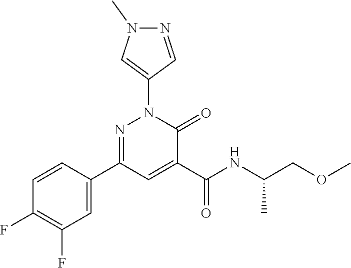 [SMILES: COC[C@H](C)NC(=O)c1cc(-c2ccc(F)c(F)c2)nn(-c2cnn(C)c2)c1=O] |
| 104 | 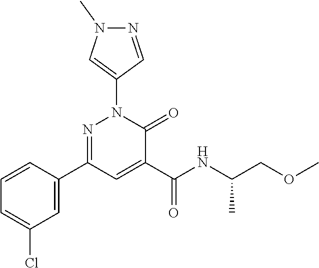 [SMILES: COC[C@H](C)NC(=O)c1cc(-c2cccc(Cl)c2)nn(-c2cnn(C)c2)c1=O] |
| 105 | 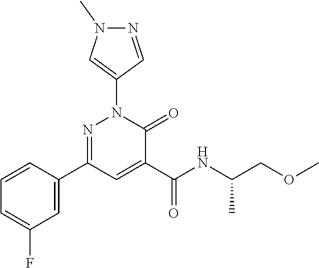 [SMILES: COC[C@H](C)NC(=O)c1cc(-c2cccc(F)c2)nn(-c2cnn(C)c2)c1=O] |
| 106 | 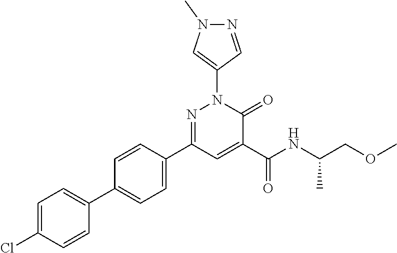 [SMILES: COC[C@H](C)NC(=O)c1cc(-c2ccc(-c3ccc(Cl)cc3)cc2)nn(-c2cnn(C)c2)c1=O] |
| 107 | 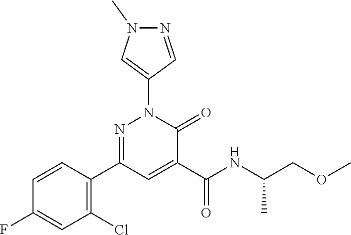 [SMILES: COC[C@H](C)NC(=O)c1cc(-c2ccc(F)cc2Cl)nn(-c2cnn(C)c2)c1=O] |
| 108 | 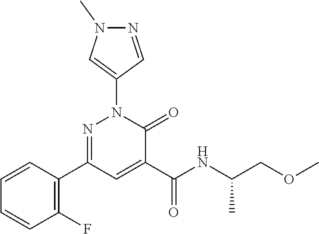 [SMILES: COC[C@H](C)NC(=O)c1cc(-c2ccccc2F)nn(-c2cnn(C)c2)c1=O] |
| 109 | 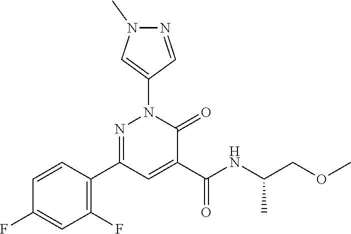 [SMILES: COC[C@H](C)NC(=O)c1cc(-c2ccc(F)cc2F)nn(-c2cnn(C)c2)c1=O] |
| 110 | 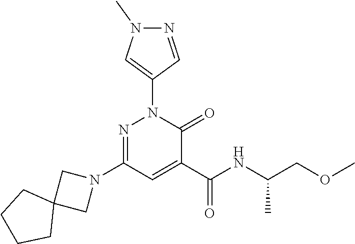 [SMILES: COC[C@H](C)NC(=O)c1cc(N2CC3(CCCC3)C2)nn(-c2cnn(C)c2)c1=O] |
| 111 | 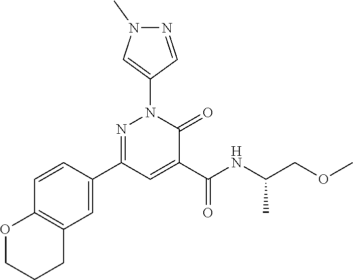 [SMILES: COC[C@H](C)NC(=O)c1cc(-c2ccc3c(c2)CCCO3)nn(-c2cnn(C)c2)c1=O] |
| 112 | 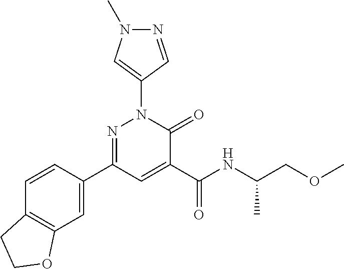 [SMILES: COC[C@H](C)NC(=O)c1cc(-c2ccc3c(c2)OCC3)nn(-c2cnn(C)c2)c1=O] |
| 113 | 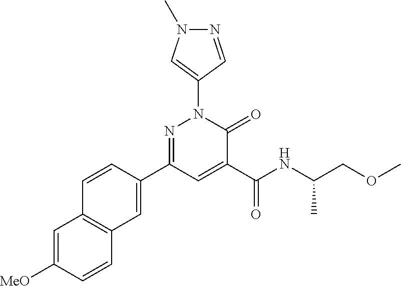 [SMILES: COC[C@H](C)NC(=O)c1cc(-c2ccc3cc(OC)ccc3c2)nn(-c2cnn(C)c2)c1=O] |
| 114 | 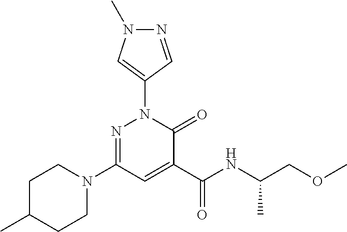 |
| 115 | 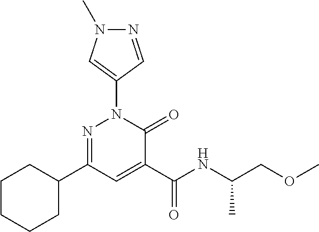 [SMILES: COC[C@H](C)NC(=O)c1cc(C2CCCCC2)nn(-c2cnn(C)c2)c1=O] |
| 116 | 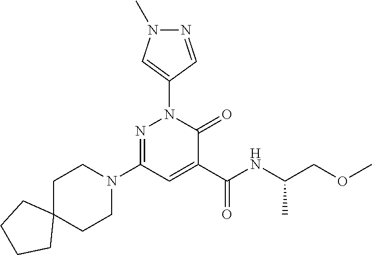 [SMILES: COC[C@H](C)NC(=O)c1cc(N2CCC3(CCCC3)CC2)nn(-c2cnn(C)c2)c1=O] |
| 117 | 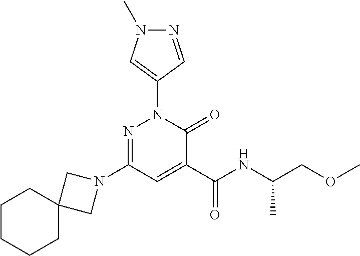 [SMILES: COC[C@H](C)NC(=O)c1cc(N2CC3(CCCCC3)C2)nn(-c2cnn(C)c2)c1=O] |
| 118 | 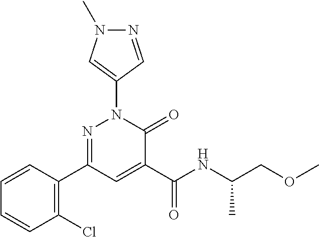 [SMILES: COC[C@H](C)NC(=O)c1cc(-c2ccccc2Cl)nn(-c2cnn(C)c2)c1=O] |
| 119 | 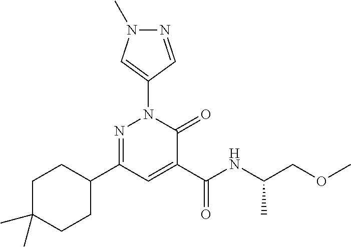 |
| 120 | 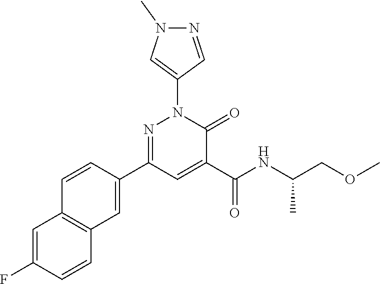 [SMILES: COC[C@H](C)NC(=O)c1cc(-c2ccc3cc(F)ccc3c2)nn(-c2cnn(C)c2)c1=O] |
| 121 | 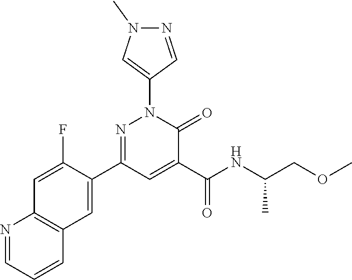 [SMILES: COC[C@H](C)NC(=O)c1cc(-c2cc3cccnc3cc2F)nn(-c2cnn(C)c2)c1=O] |
| 122 | 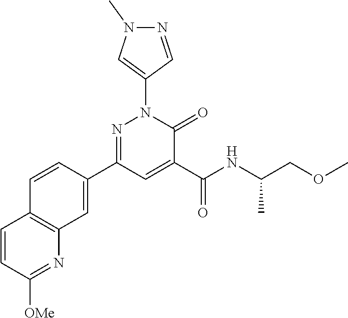 [SMILES: COC[C@H](C)NC(=O)c1cc(-c2ccc3ccc(OC)nc3c2)nn(-c2cnn(C)c2)c1=O] |
| 123 | 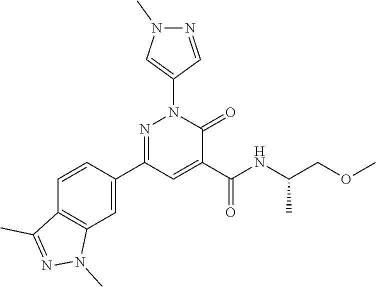 |
| 124 | 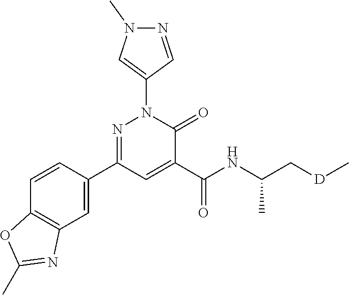 |
| 125 | 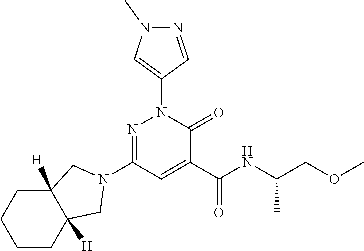 [SMILES: COC[C@H](C)NC(=O)c1cc(N2C[C@H]3CCCC[C@H]3C2)nn(-c2cnn(C)c2)c1=O] |
| 126 | 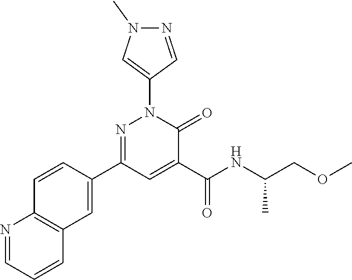 [SMILES: COC[C@H](C)NC(=O)c1cc(-c2ccc3ncccc3c2)nn(-c2cnn(C)c2)c1=O] |
| 127 | 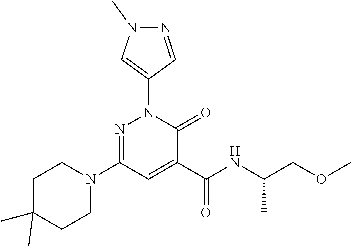 |
| 128 | 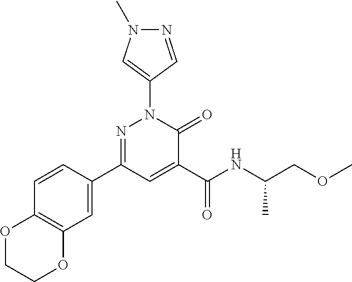 [SMILES: COC[C@H](C)NC(=O)c1cc(-c2ccc3c(c2)OCCO3)nn(-c2cnn(C)c2)c1=O] |
| 129 | 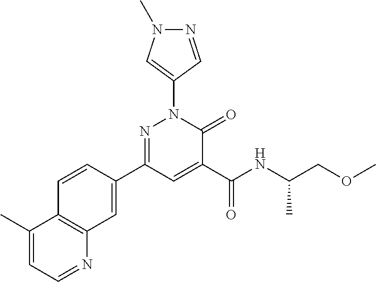 |
| 130 | 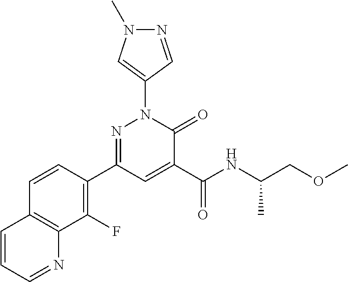 [SMILES: COC[C@H](C)NC(=O)c1cc(-c2ccc3cccnc3c2F)nn(-c2cnn(C)c2)c1=O] |
| 131 | 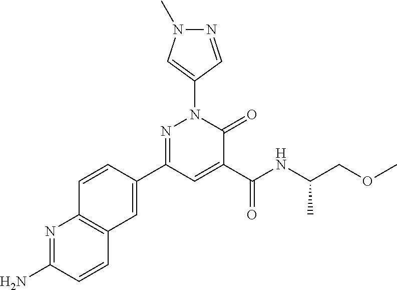 [SMILES: COC[C@H](C)NC(=O)c1cc(-c2ccc3nc(N)ccc3c2)nn(-c2cnn(C)c2)c1=O] |
| 132 | 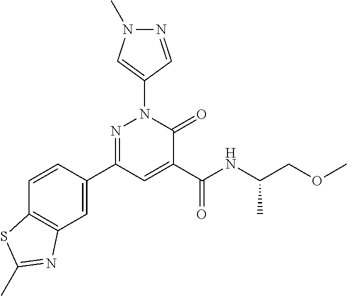 |
| 133 | 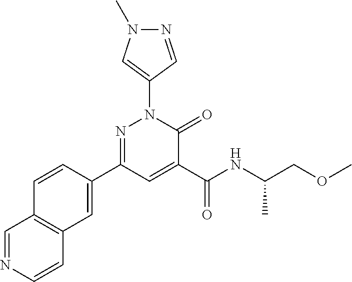 [SMILES: COC[C@H](C)NC(=O)c1cc(-c2ccc3cnccc3c2)nn(-c2cnn(C)c2)c1=O] |
| 134 | 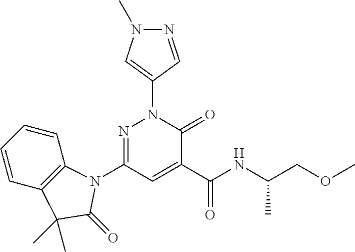 [SMILES: COC[C@H](C)NC(=O)c1cc(N2C(=O)C(C)(C)c3ccccc32)nn(-c2cnn(C)c2)c1=O] |
| 135 | 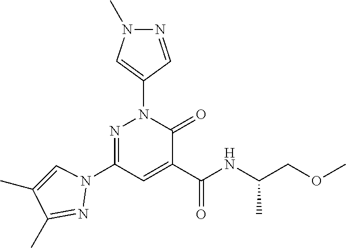 |
| 136 | 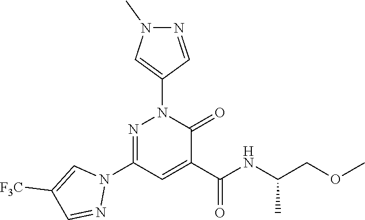 [SMILES: COC[C@H](C)NC(=O)c1cc(-n2cc(C(F)(F)F)cn2)nn(-c2cnn(C)c2)c1=O] |
| 137 | 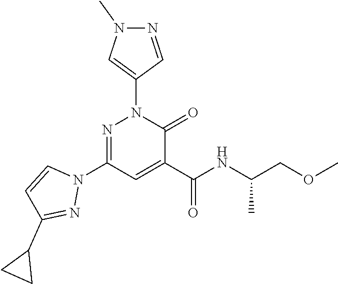 [SMILES: COC[C@H](C)NC(=O)c1cc(-n2ccc(C3CC3)n2)nn(-c2cnn(C)c2)c1=O] |
| 138 | 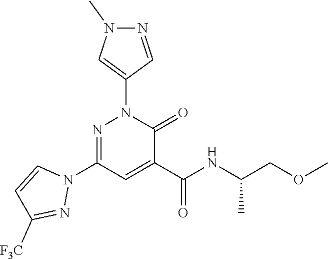 [SMILES: COC[C@H](C)NC(=O)c1cc(-n2ccc(C(F)(F)F)n2)nn(-c2cnn(C)c2)c1=O] |
| 139 | 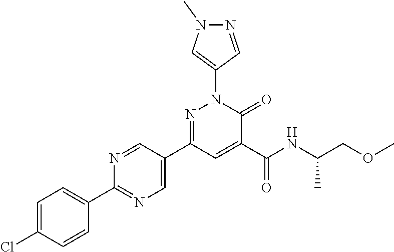 [SMILES: COC[C@H](C)NC(=O)c1cc(-c2cnc(-c3ccc(Cl)cc3)nc2)nn(-c2cnn(C)c2)c1=O] |
| 140 | 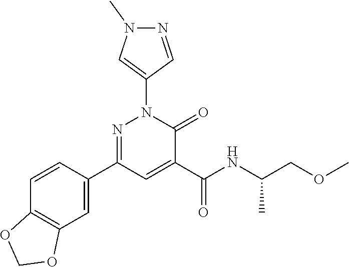 [SMILES: COC[C@H](C)NC(=O)c1cc(-c2ccc3c(c2)OCO3)nn(-c2cnn(C)c2)c1=O] |
| 141 | 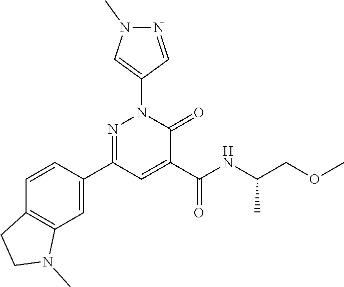 [SMILES: COC[C@H](C)NC(=O)c1cc(-c2ccc3c(c2)N(C)CC3)nn(-c2cnn(C)c2)c1=O] |
| 142 | 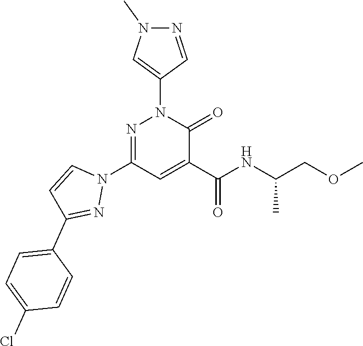 [SMILES: COC[C@H](C)NC(=O)c1cc(-n2ccc(-c3ccc(Cl)cc3)n2)nn(-c2cnn(C)c2)c1=O] |
| 143 | 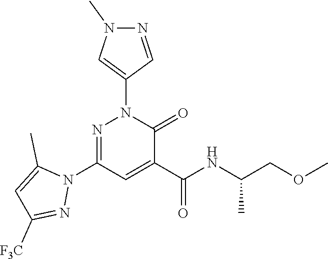 [SMILES: COC[C@H](C)NC(=O)c1cc(-n2nc(C(F)(F)F)cc2C)nn(-c2cnn(C)c2)c1=O] |
| 144 | 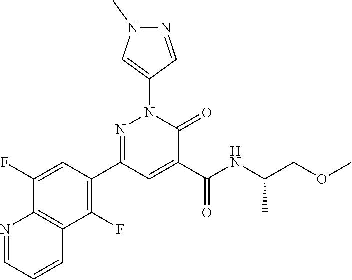 [SMILES: COC[C@H](C)NC(=O)c1cc(-c2cc(F)c3ncccc3c2F)nn(-c2cnn(C)c2)c1=O] |
| 145 | 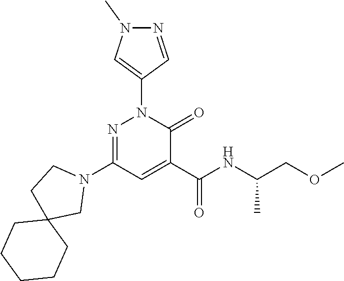 [SMILES: COC[C@H](C)NC(=O)c1cc(N2CCC3(CCCCC3)C2)nn(-c2cnn(C)c2)c1=O] |
| 146 | 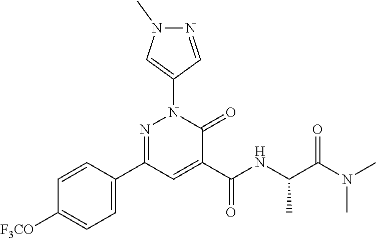 [SMILES: C[C@H](NC(=O)c1cc(-c2ccc(OC(F)(F)F)cc2)nn(-c2cnn(C)c2)c1=O)C(=O)N(C)C] |
| 147 | 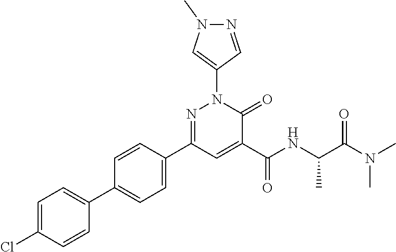 [SMILES: C[C@H](NC(=O)c1cc(-c2ccc(-c3ccc(Cl)cc3)cc2)nn(-c2cnn(C)c2)c1=O)C(=O)N(C)C] |
| 148 | 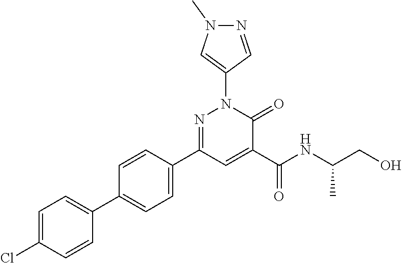 [SMILES: C[C@@H](CO)NC(=O)c1cc(-c2ccc(-c3ccc(Cl)cc3)cc2)nn(-c2cnn(C)c2)c1=O] |
| 149 | 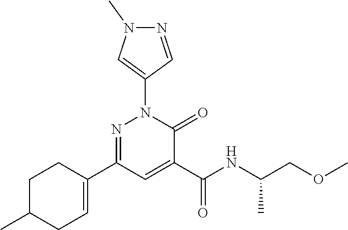 |
| 150 | 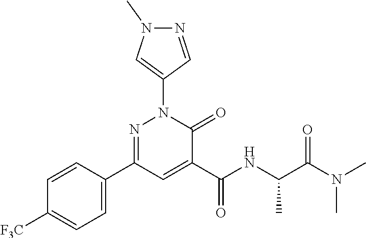 [SMILES: C[C@H](NC(=O)c1cc(-c2ccc(C(F)(F)F)cc2)nn(-c2cnn(C)c2)c1=O)C(=O)N(C)C] |
| 151 | 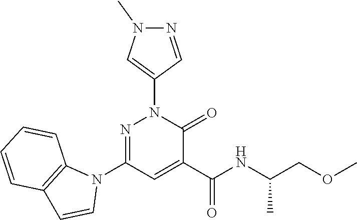 [SMILES: COC[C@H](C)NC(=O)c1cc(-n2ccc3ccccc32)nn(-c2cnn(C)c2)c1=O] |
| 152 | 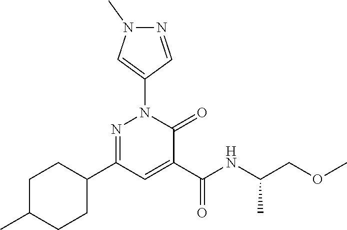 |
| 153 | 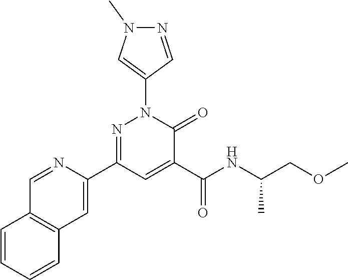 [SMILES: COC[C@H](C)NC(=O)c1cc(-c2cc3ccccc3cn2)nn(-c2cnn(C)c2)c1=O] |
| 154 | 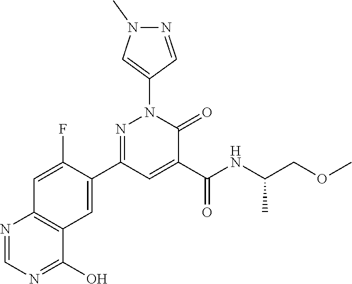 [SMILES: COC[C@H](C)NC(=O)c1cc(-c2cc3c(O)ncnc3cc2F)nn(-c2cnn(C)c2)c1=O] |
| 155 | 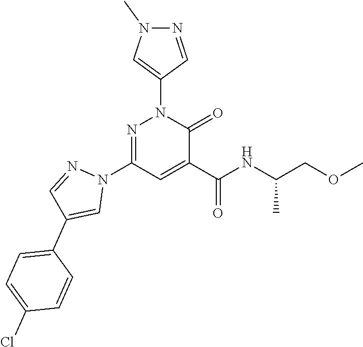 [SMILES: COC[C@H](C)NC(=O)c1cc(-n2cc(-c3ccc(Cl)cc3)cn2)nn(-c2cnn(C)c2)c1=O] |
| 156 | 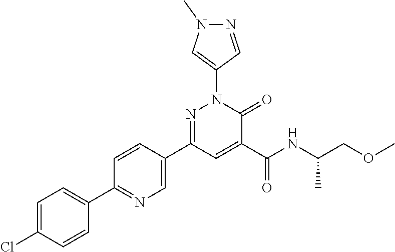 [SMILES: COC[C@H](C)NC(=O)c1cc(-c2ccc(-c3ccc(Cl)cc3)nc2)nn(-c2cnn(C)c2)c1=O] |
| 157 | 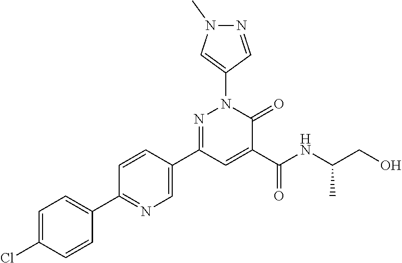 [SMILES: C[C@@H](CO)NC(=O)c1cc(-c2ccc(-c3ccc(Cl)cc3)nc2)nn(-c2cnn(C)c2)c1=O] |
| 158 | 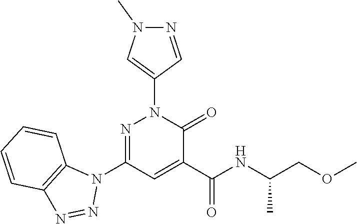 [SMILES: COC[C@H](C)NC(=O)c1cc(-n2nnc3ccccc32)nn(-c2cnn(C)c2)c1=O] |
| 159 | 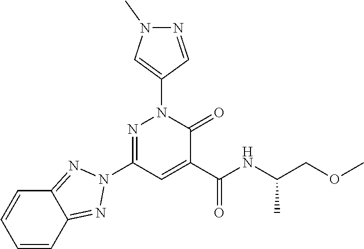 [SMILES: COC[C@H](C)NC(=O)c1cc(-n2nc3ccccc3n2)nn(-c2cnn(C)c2)c1=O] |
| 160 | 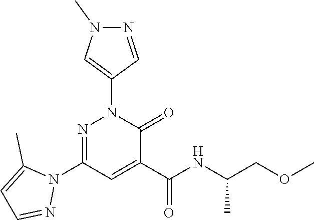 [SMILES: COC[C@H](C)NC(=O)c1cc(-n2nccc2C)nn(-c2cnn(C)c2)c1=O] |
| 161 | 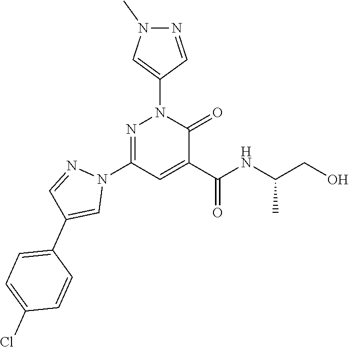 [SMILES: C[C@@H](CO)NC(=O)c1cc(-n2cc(-c3ccc(Cl)cc3)cn2)nn(-c2cnn(C)c2)c1=O] |
| 162 | 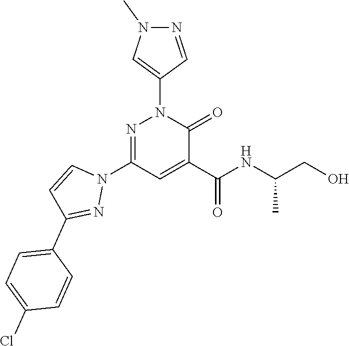 [SMILES: C[C@@H](CO)NC(=O)c1cc(-n2ccc(-c3ccc(Cl)cc3)n2)nn(-c2cnn(C)c2)c1=O] |
| 163 | 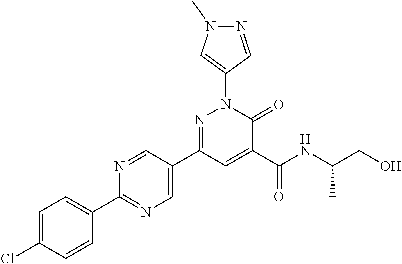 [SMILES: C[C@@H](CO)NC(=O)c1cc(-c2cnc(-c3ccc(Cl)cc3)nc2)nn(-c2cnn(C)c2)c1=O] |
| 164 | 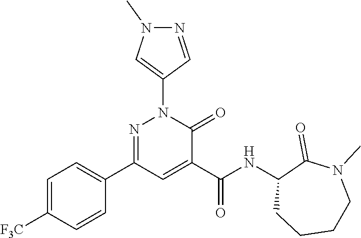 [SMILES: CN1CCCC[C@H](NC(=O)c2cc(-c3ccc(C(F)(F)F)cc3)nn(-c3cnn(C)c3)c2=O)C1=O] |
| 165 | 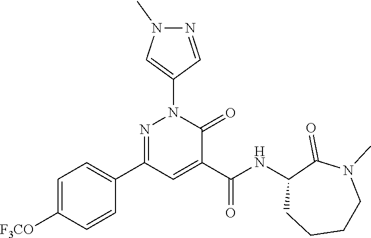 [SMILES: CN1CCCC[C@H](NC(=O)c2cc(-c3ccc(OC(F)(F)F)cc3)nn(-c3cnn(C)c3)c2=O)C1=O] |
| 166 | 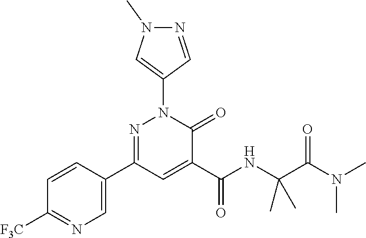 [SMILES: CN(C)C(=O)C(C)(C)NC(=O)c1cc(-c2ccc(C(F)(F)F)nc2)nn(-c2cnn(C)c2)c1=O] |
| 167 | 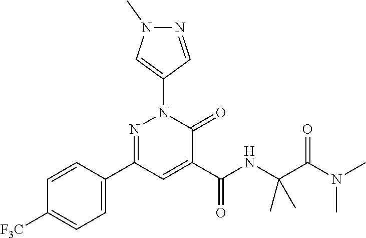 [SMILES: CN(C)C(=O)C(C)(C)NC(=O)c1cc(-c2ccc(C(F)(F)F)cc2)nn(-c2cnn(C)c2)c1=O] |
| 168 | 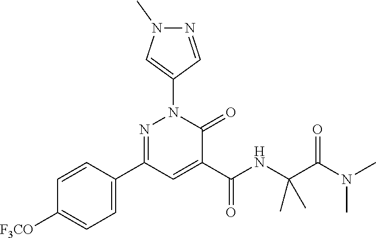 [SMILES: CN(C)C(=O)C(C)(C)NC(=O)c1cc(-c2ccc(OC(F)(F)F)cc2)nn(-c2cnn(C)c2)c1=O] |
| 169 | 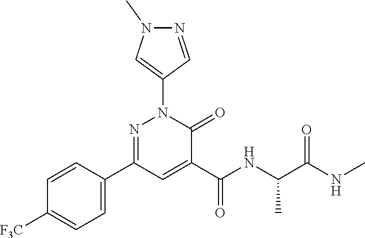 [SMILES: CNC(=O)[C@H](C)NC(=O)c1cc(-c2ccc(C(F)(F)F)cc2)nn(-c2cnn(C)c2)c1=O] |
| 170 | 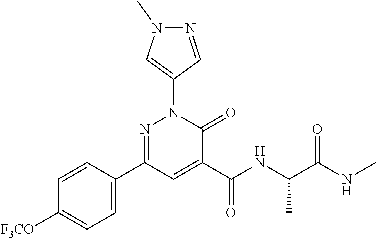 [SMILES: CNC(=O)[C@H](C)NC(=O)c1cc(-c2ccc(OC(F)(F)F)cc2)nn(-c2cnn(C)c2)c1=O] |
| 171 | 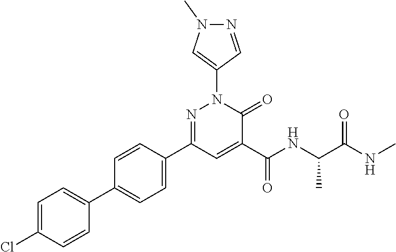 [SMILES: CNC(=O)[C@H](C)NC(=O)c1cc(-c2ccc(-c3ccc(Cl)cc3)cc2)nn(-c2cnn(C)c2)c1=O] |
| 172 | 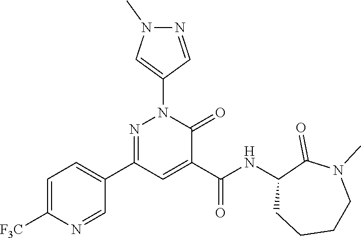 [SMILES: CN1CCCC[C@H](NC(=O)c2cc(-c3ccc(C(F)(F)F)nc3)nn(-c3cnn(C)c3)c2=O)C1=O] |
| 173 | 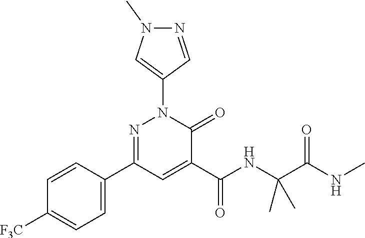 [SMILES: CNC(=O)C(C)(C)NC(=O)c1cc(-c2ccc(C(F)(F)F)cc2)nn(-c2cnn(C)c2)c1=O] |
| 174 | 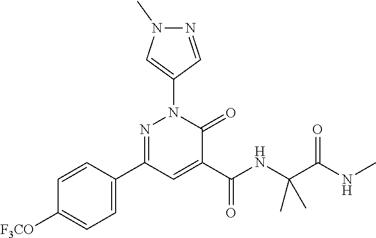 [SMILES: CNC(=O)C(C)(C)NC(=O)c1cc(-c2ccc(OC(F)(F)F)cc2)nn(-c2cnn(C)c2)c1=O] |
| 175 | 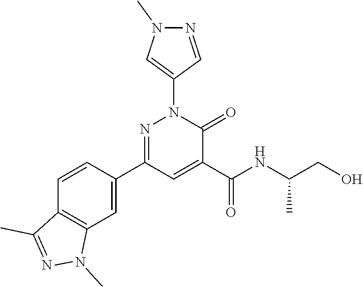 [SMILES: Cc1nn(C)c2cc(-c3cc(C(=O)N[C@@H](C)CO)c(=O)n(-c4cnn(C)c4)n3)ccc12] |
| 176 | 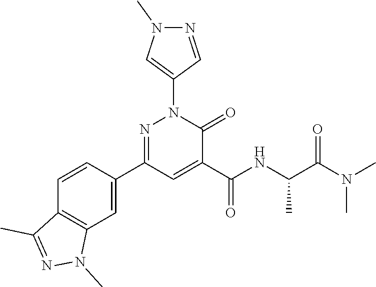 |
| 177 | 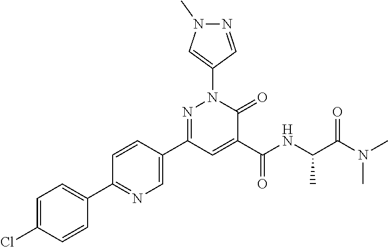 [SMILES: C[C@H](NC(=O)c1cc(-c2ccc(-c3ccc(Cl)cc3)nc2)nn(-c2cnn(C)c2)c1=O)C(=O)N(C)C] |
| 178 | 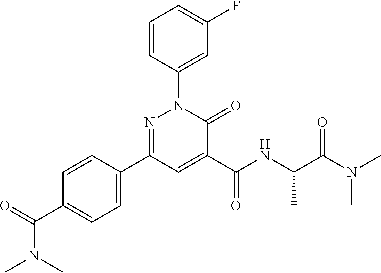 [SMILES: C[C@H](NC(=O)c1cc(-c2ccc(C(=O)N(C)C)cc2)nn(-c2cccc(F)c2)c1=O)C(=O)N(C)C] |
| 179 | 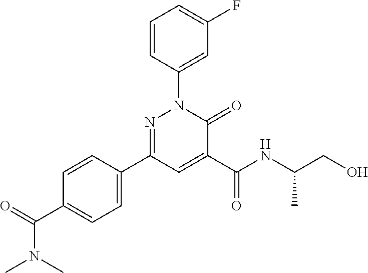 [SMILES: C[C@@H](CO)NC(=O)c1cc(-c2ccc(C(=O)N(C)C)cc2)nn(-c2cccc(F)c2)c1=O] |
| 180 | 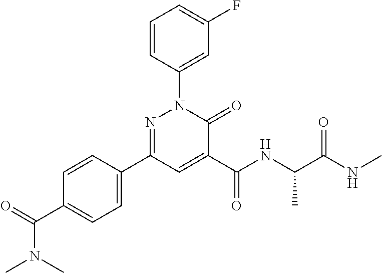 [SMILES: CNC(=O)[C@H](C)NC(=O)c1cc(-c2ccc(C(=O)N(C)C)cc2)nn(-c2cccc(F)c2)c1=O] |
| 181 | 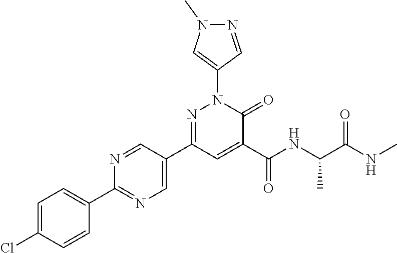 [SMILES: CNC(=O)[C@H](C)NC(=O)c1cc(-c2cnc(-c3ccc(Cl)cc3)nc2)nn(-c2cnn(C)c2)c1=O] |
| 182 | 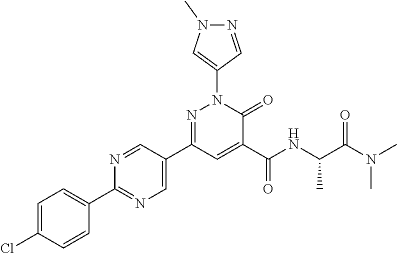 [SMILES: C[C@H](NC(=O)c1cc(-c2cnc(-c3ccc(Cl)cc3)nc2)nn(-c2cnn(C)c2)c1=O)C(=O)N(C)C] |
| 183 | 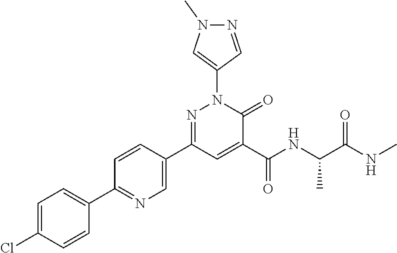 [SMILES: CNC(=O)[C@H](C)NC(=O)c1cc(-c2ccc(-c3ccc(Cl)cc3)nc2)nn(-c2cnn(C)c2)c1=O] |
| 184 | 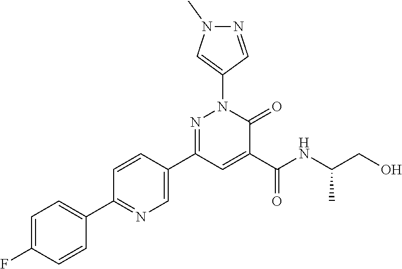 [SMILES: C[C@@H](CO)NC(=O)c1cc(-c2ccc(-c3ccc(F)cc3)nc2)nn(-c2cnn(C)c2)c1=O] |
| 185 | 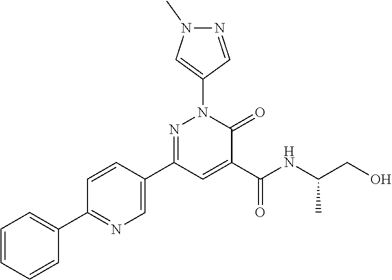 [SMILES: C[C@@H](CO)NC(=O)c1cc(-c2ccc(-c3ccccc3)nc2)nn(-c2cnn(C)c2)c1=O] |
| 186 | 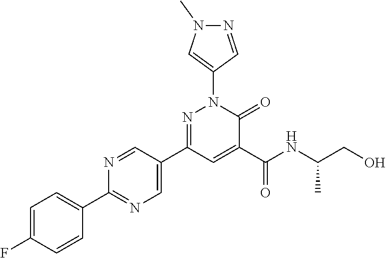 [SMILES: C[C@@H](CO)NC(=O)c1cc(-c2cnc(-c3ccc(F)cc3)nc2)nn(-c2cnn(C)c2)c1=O] |
| 187 | 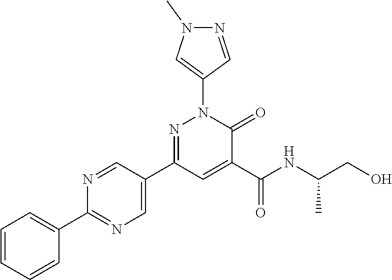 [SMILES: C[C@@H](CO)NC(=O)c1cc(-c2cnc(-c3ccccc3)nc2)nn(-c2cnn(C)c2)c1=O] |
| 188 | 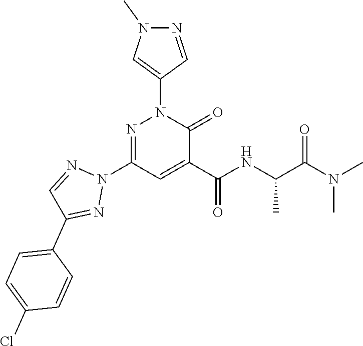 [SMILES: C[C@H](NC(=O)c1cc(-n2ncc(-c3ccc(Cl)cc3)n2)nn(-c2cnn(C)c2)c1=O)C(=O)N(C)C] |
| 189 | 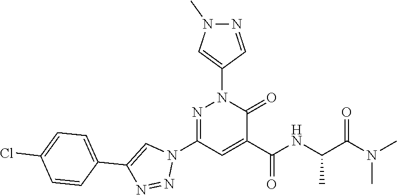 [SMILES: C[C@H](NC(=O)c1cc(-n2cc(-c3ccc(Cl)cc3)nn2)nn(-c2cnn(C)c2)c1=O)C(=O)N(C)C] |
| 190 | 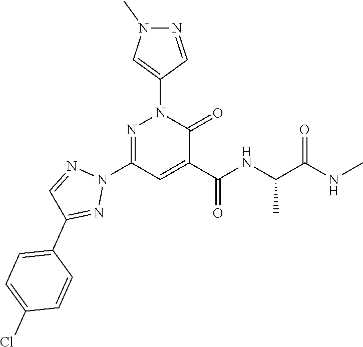 [SMILES: CNC(=O)[C@H](C)NC(=O)c1cc(-n2ncc(-c3ccc(Cl)cc3)n2)nn(-c2cnn(C)c2)c1=O] |
| 191 | 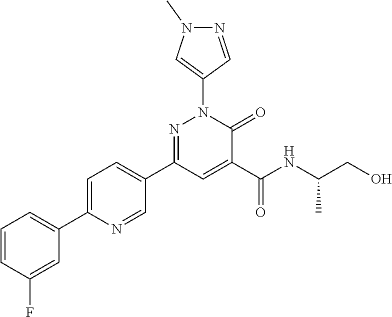 [SMILES: C[C@@H](CO)NC(=O)c1cc(-c2ccc(-c3cccc(F)c3)nc2)nn(-c2cnn(C)c2)c1=O] |
| 192 | 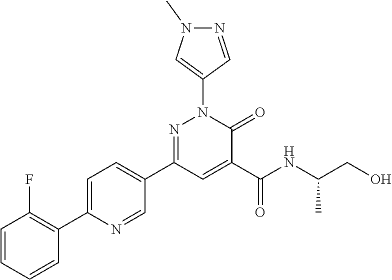 [SMILES: C[C@@H](CO)NC(=O)c1cc(-c2ccc(-c3ccccc3F)nc2)nn(-c2cnn(C)c2)c1=O] |
| 193 | 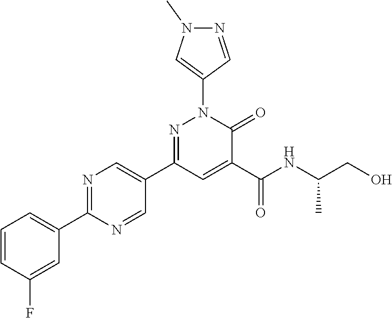 [SMILES: C[C@@H](CO)NC(=O)c1cc(-c2cnc(-c3cccc(F)c3)nc2)nn(-c2cnn(C)c2)c1=O] |
| 194 | 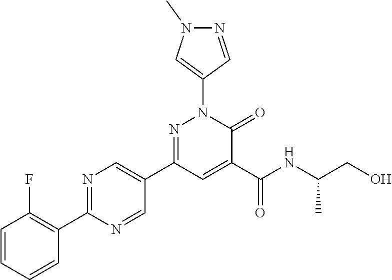 [SMILES: C[C@@H](CO)NC(=O)c1cc(-c2cnc(-c3ccccc3F)nc2)nn(-c2cnn(C)c2)c1=O] |
| 195 | 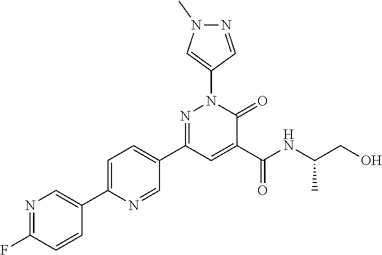 [SMILES: C[C@@H](CO)NC(=O)c1cc(-c2ccc(-c3ccc(F)nc3)nc2)nn(-c2cnn(C)c2)c1=O] |
| 196 | 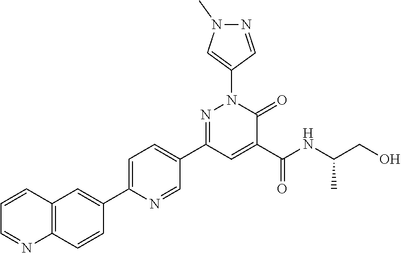 [SMILES: C[C@@H](CO)NC(=O)c1cc(-c2ccc(-c3ccc4ncccc4c3)nc2)nn(-c2cnn(C)c2)c1=O] |
| 197 | 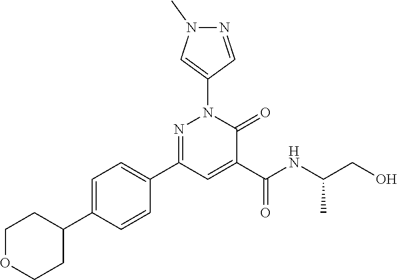 [SMILES: C[C@@H](CO)NC(=O)c1cc(-c2ccc(C3CCOCC3)cc2)nn(-c2cnn(C)c2)c1=O] |
| 198 | 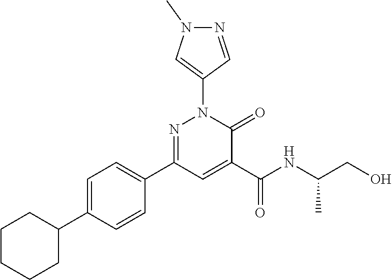 [SMILES: C[C@@H](CO)NC(=O)c1cc(-c2ccc(C3CCCCC3)cc2)nn(-c2cnn(C)c2)c1=O] |
| 199 | 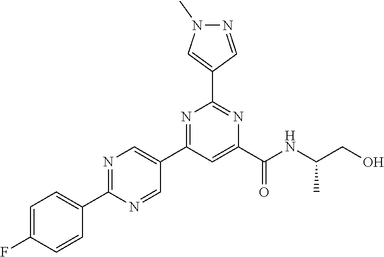 [SMILES: C[C@@H](CO)NC(=O)c1cc(-c2cnc(-c3ccc(F)cc3)nc2)nc(-c2cnn(C)c2)n1] |
| 200 | 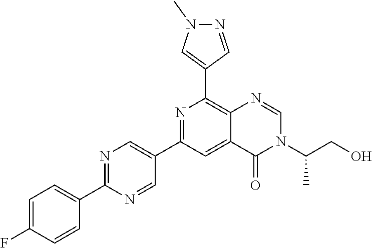 [SMILES: C[C@@H](CO)n1cnc2c(-c3cnn(C)c3)nc(-c3cnc(-c4ccc(F)cc4)nc3)cc2c1=O] |
| 201 | 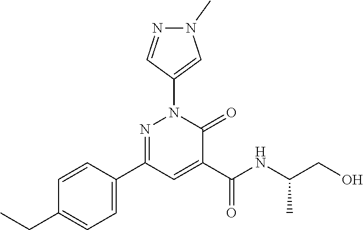 [SMILES: CCc1ccc(-c2cc(C(=O)N[C@@H](C)CO)c(=O)n(-c3cnn(C)c3)n2)cc1] |
| 202 | 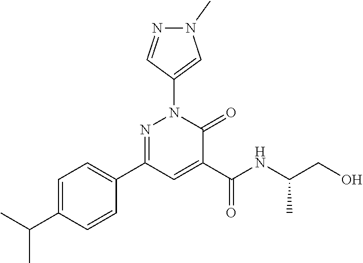 [SMILES: CC(C)c1ccc(-c2cc(C(=O)N[C@@H](C)CO)c(=O)n(-c3cnn(C)c3)n2)cc1] |
| 203 | 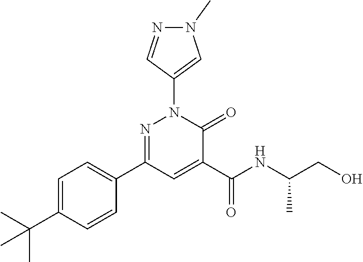 [SMILES: C[C@@H](CO)NC(=O)c1cc(-c2ccc(C(C)(C)C)cc2)nn(-c2cnn(C)c2)c1=O] |
| 204 | 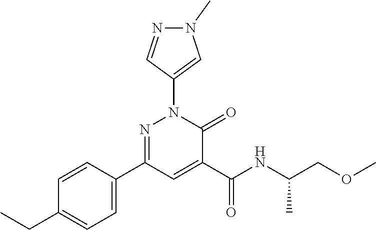 [SMILES: CCc1ccc(-c2cc(C(=O)N[C@@H](C)COC)c(=O)n(-c3cnn(C)c3)n2)cc1] |
| 205 | 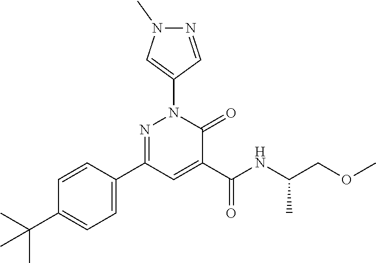 [SMILES: COC[C@H](C)NC(=O)c1cc(-c2ccc(C(C)(C)C)cc2)nn(-c2cnn(C)c2)c1=O] |
| 206 | 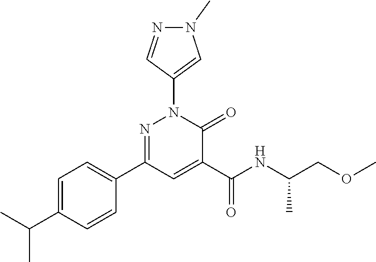 [SMILES: COC[C@H](C)NC(=O)c1cc(-c2ccc(C(C)C)cc2)nn(-c2cnn(C)c2)c1=O] |
| 207 | 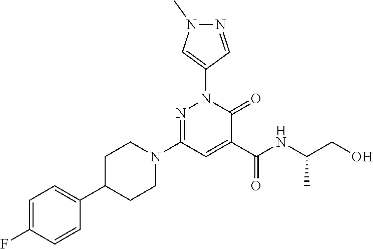 [SMILES: C[C@@H](CO)NC(=O)c1cc(N2CCC(c3ccc(F)cc3)CC2)nn(-c2cnn(C)c2)c1=O] |
| 208 | 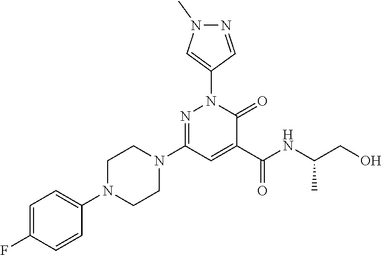 [SMILES: C[C@@H](CO)NC(=O)c1cc(N2CCN(c3ccc(F)cc3)CC2)nn(-c2cnn(C)c2)c1=O] |
| 209 | 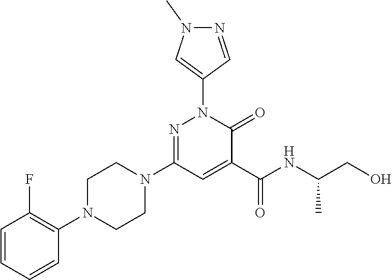 [SMILES: C[C@@H](CO)NC(=O)c1cc(N2CCN(c3ccccc3F)CC2)nn(-c2cnn(C)c2)c1=O] |
| 210 | 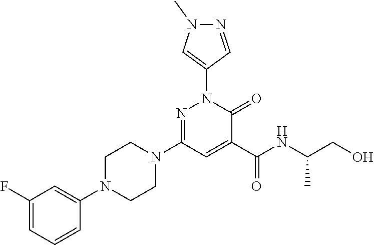 [SMILES: C[C@@H](CO)NC(=O)c1cc(N2CCN(c3cccc(F)c3)CC2)nn(-c2cnn(C)c2)c1=O] |

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

The compounds of Formula (I) can have any stereochemical configuration at the sp³ carbon atoms. In some cases, the compounds of the disclosure are optically pure. As used herein, “optically pure” refers to the presence of only one enantiomer of a compound if multiple stereochemical configurations can exist. In various cases, the chiral moieties present in the compounds of the disclosure are derived from either natural or unnatural amino acids or saccharides.

Unless otherwise indicated, all tautomeric forms of the compounds of the disclosure are within the scope of the disclosure.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure. Such compounds are useful, for example, as analytical tools or probes in biological assays. Such compounds, especially deuterium analogs, can also be therapeutically useful. Thus, further disclosed herein are deuterated compounds or salts of Formula (I), in which one or more isotopes of hydrogen have been replaced with deuterium.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

As used herein, “alkyl” refers to straight chained and branched saturated hydrocarbon groups containing one to thirty carbon atoms, for example, one to six carbon atoms (e.g., 1, 2, 3, 4, 5, or 6). The term Cₙ means the alkyl group has “n” carbon atoms. For example, C₃ alkyl refers to an alkyl group that has 3 carbon atoms. C₁₋₆alkyl refers to an alkyl group having a number of carbon atoms encompassing the entire range (i.e., 1 to 6 carbon atoms), as well as all subgroups (e.g., 1-2, 1-3, 1-4, 1-5, 1-6, 2-3, 2-4, 2-5, 2-6, 3-4, 3-5, 3-6, 4-5, 4-6, 5-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, “alkylene” refers to a bivalent saturated aliphatic radical. The term C₀ means the alkylene group has “n” carbon atoms. For example, C₁₋₆alkylene refers to an alkylene group having a number of carbon atoms encompassing the entire range, as well as all subgroups, as previously described for “alkyl” groups.

As used herein, the term “haloalkyl” refers to an alkyl group in which one or more of the hydrogen atoms are replaced by halogen. Such groups include but are not limited to, chloromethyl, fluoromethyl, difluoromethyl, trifluoromethyl, 1,1-difluoroethyl, 2-fluoroethyl, 1-chloro-2-fluoromethyl and 2-fluoroisobutyl. A haloalkyl may be further substituted or unsubstituted, and some cases relate to a haloalkyl having e.g., 1 to 6 carbon atoms, such as C₁₋₆ haloalkyl.

As used herein, the term “hydroxyalkyl” refers to an alkyl group in which one or more of the hydrogen atoms are replaced by a hydroxyl group (OH). Such groups include but are not limited to, hydroxymethyl, hydroxyethyl, and the like. A hydroxyalkyl may be further substituted or unsubstituted, and some cases relate to a hydroxyalkyl having e.g., 1 to 6 carbon atoms, such as C₁₋₆ hydroxyalkyl.

As used herein, the term “cycloalkyl” refers to an aliphatic cyclic hydrocarbon group containing three to eight carbon atoms (e.g., 3, 4, 5, 6, 7, or 8 carbon atoms). The term Cₙ means the cycloalkyl group has “n” carbon atoms. For example, C₅ cycloalkyl refers to a cycloalkyl group that has 5 carbon atoms in the ring. C₃—C cycloalkyl refers to cycloalkyl groups having a number of carbon atoms encompassing the entire range (e.g., 3 to 8 carbon atoms), as well as all subgroups (e.g., 3-4, 3-5, 3-6, 3-7, 3-8, 4-5, 4-6, 4-7, 4-8, 5-6, 5-7, 5-8, 6-7, 6-8, 7-8, 3, 4, 5, 6, 7, and 8 carbon atoms). Nonlimiting examples of cycloalkyl groups include cyclopropyl, cyclobutyl, cyclopentyl, cyclohexyl, cycloheptyl, and cyclooctyl. The cycloalkyl groups described herein can be isolated or fused to another cycloalkyl group, a heterocycloalkyl group, an aryl group and/or a heteroaryl group. When a cycloalkyl group is fused to another cycloalkyl group, then each of the cycloalkyl groups can contain three to eight carbon atoms unless specified otherwise. Unless otherwise indicated, a cycloalkyl group can be unsubstituted or substituted.

The term “heterocyclyl” as used herein refers to a non-aromatic monocyclic, fused, spiro or bridged ring system which can be saturated or contain one or more units of unsaturation, having 4 to 12 total ring atoms in which 1-3 (e.g., one to three, or one, two, or three) ring atoms is a heteroatom selected from, N, S, and O. In cases where the heterocyclyl is spiro, the group can comprise, e.g., a 4-membered ring and spiro 5-membered ring; a 4-membered ring and spiro 6-membered ring; a 4-membered ring and spiro 7-membered ring; a 5-membered ring and spiro 6-membered ring; a 5-membered ring and spiro 7-membered ring; a 6-membered ring and spiro 6-membered ring; a 6-membered ring and spiro 7-membered ring; or a 7-membered ring and spiro 7-membered ring; and each ring of the spiro system may contain at least one ring heteroatom, or only one ring may contain at least one ring heteroatom and the other ring can be carbocyclic. In some cases, the heterocyclyl comprises 5-6 ring members. In some cases, the heterocyclyl comprises 5 ring members. In some cases, the heterocyclyl comprises 6 ring members. Examples of heterocyclyl groups include, but are not limited to, oxetanyl, azetidinyl, piperidinyl, piperazinyl, pyrrolidinyl, pyrazolidinyl, imidazolidinyl, azepanyl, diazepanyl, triazepanyl, oxazolidinyl, isoxazolidinyl, thiazolidinyl, isothiazolidinyl, oxazepanyl, thiazepanyl, tetrahydrofuranyl, tetrahydrothiophenyl, morpholino (including, for example, 3-morpholino, 4-morpholino), 2-thiomorpholino, 3-thiomorpholino, 4-thiomorpholino, 1-pyrrolidinyl, 2-pyrrolidinyl, 3-pyrrolidinyl, pyrrolidin-2-one, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, and 1,3-dihydro-imidazol-2-onyl. In some cases, heterocyclyl is oxetanyl, azetidinyl, diazepanyl (such as 1,4-diazepanyl), azepanyl, piperidinyl (such as 1-piperidinyl, 2-piperidinyl, 3-piperidinyl, or 4-piperidinyl), or pyrrolidinyl (such as 1-pyrrolidinyl, 2-pyrrolidinyl, or 3-pyrrolidinyl). In some cases, heterocyclyl is oxetanyl, azetidinyl, diazepanyl or azepanyl. In some cases, heterocyclyl is diazepanyl or azepanyl. In some cases, heterocyclyl is 1,4-diazepanyl or azepanyl. In some cases, heterocyclyl is 1,4-diazepanyl. In some cases, heterocyclyl is azepanyl. A heterocyclyl ring is unsubstituted or substituted as described herein.

As used herein, the term “aryl” refers to a monocyclic or bicyclic aromatic group, having 6 to 10 ring atoms. Unless otherwise indicated, an aryl group can be unsubstituted or substituted. Aryl groups can be isolated (e.g., phenyl) or fused to another aryl group (e.g., naphthyl, or anthracenyl) or cyclic group (e.g., indene, dihydroindene).

As used herein, the term “heteroaryl” refers to a heterocycyl group that is aromatic, having five to twelve total ring atoms (e.g., a monocyclic aromatic ring with 5-6 total ring atoms), and containing 1-3 heteroatoms selected from nitrogen, oxygen, and sulfur atom in the aromatic ring. Unless otherwise indicated, a heteroaryl group can be unsubstituted or substituted. Heteroaryl groups can be isolated (e.g., pyridyl) or fused to another heteroaryl group (e.g., purinyl), a cycloalkyl group (e.g., tetrahydroquinolinyl), a non-aromatic heterocyclyl group (e.g., dihydronaphthyridinyl), and/or an aryl group (e.g., benzothiazolyl and quinolyl).

Examples of heteroaryl groups include, but are not limited to, pyrazolyl, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl. When a heteroaryl group is fused to another heteroaryl group, then each ring can contain five or six total ring atoms and one to three heteroatoms in its aromatic ring.

As used herein, the term “alkoxy” as used herein refers to a “—O-alkyl” group. The alkoxy group can be unsubstituted or substituted.

As used herein, the term “haloalkoxy” as used herein refers to a “—O-haloalkyl” group. The haloalkoxy group can be unsubstituted or substituted.

As used herein, the term “halo” refers to a fluoro (F), chloro (Cl), bromo (Br), or iodo (I) group.

A “substituted” functional group (e.g., a substituted heterocyclyl, aryl, or heteroaryl refers to an alkyl, heterocyclyl, aryl, or heteroaryl) is a functional, group having at least one hydrogen radical that is substituted with a non-hydrogen radical (i.e., a substituent). Examples of non-hydrogen radicals (or substituents) include, but are not limited to, alkyl, cycloalkyl, alkenyl, cycloalkenyl, alkynyl, ether, aryl, heteroaryl, heterocycloalkyl, hydroxyl, oxy (or oxo), alkoxyl, ester, thioester, acyl, carboxyl, cyano, nitro, amino, sulfhydryl, and halo. When a substituted alkyl group includes more than one non-hydrogen radical, the substituents can be bound to the same carbon or two or more different carbon atoms. Other substitutions are discussed in relation to specific functional groups or moieties herein.

### Pharmaceutically Acceptable Salts

The compounds described herein can exist in free form, or where appropriate, as salts. Those salts that are pharmaceutically acceptable are of particular interest since they are useful in administering the compounds described herein for medical purposes. Salts that are not pharmaceutically acceptable are useful in manufacturing processes, for isolation and purification purposes, and in some cases, for use in separating stereoisomeric forms of the compounds of the disclosure or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxyl group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, although not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Formulations

Also provided herein are pharmaceutical formulations that include an effective amount of compounds of the disclosure and one or more pharmaceutically acceptable excipients. As used herein, the term “formulation” is used interchangeable with “composition.”

An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females.

As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The compounds of the disclosure can be administered alone or as part of a pharmaceutically acceptable composition or formulation. In addition, the compounds can be administered all at once, as for example, by a bolus injection, multiple times, e.g. by a series of tablets, or delivered substantially uniformly over a period of time, as for example, using transdermal delivery. It is also noted that the dose of the compound can be varied over time.

The compounds disclosed herein and other pharmaceutically active compounds, if desired, can be administered to a subject or patient by any suitable route, e.g. orally, topically, rectally, parenterally, (for example, subcutaneous injections, intravenous, intramuscular, intrasternal, and intrathecal injection or infusion techniques), or as a buccal, inhalation, or nasal spray. The administration can be to provide a systemic effect (e.g. enteral or parenteral). All methods that can be used by those skilled in the art to administer a pharmaceutically active agent are contemplated. In some cases, the disclosed formulations can be administered orally or topically.

Suitable oral compositions or formulations in accordance with the disclosure include without limitation tablets, troches, lozenges, aqueous or oily suspensions, dispersible powders or granules, emulsion, hard or soft capsules, syrups or elixirs. Compositions or formulations suitable for oral use may be prepared according to any method known to the art for the manufacture of pharmaceutical compositions.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The pharmaceutical compositions and formulations described herein may also be administered topically or transdermally, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract, e.g., can be effected in a rectal suppository formulation or in a suitable enema formulation. Dosage forms for topical or transdermal administration of a compound described herein include ointments, pastes, creams, lotions, gels, powders, solutions, sprays, inhalants, suppositories, or patches.

For topical applications, the pharmaceutical compositions may be formulated in a suitable ointment, cream, lotion, or gel, containing the active component suspended or dissolved in one or more carriers, and any needed preservatives or buffers as may be required. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Treatment

The compounds disclosed herein (e.g., the compounds of Formula (I), Table 1, and salts thereof) can modulate the aryl hydrocarbon receptor (AHR) pathway, e.g., by modulating AHR in a cell. AHR is a ligand-activated transcription factor that has been implicated in a variety of conditions, including by modulating the immune system during steady state and during infection and inflammation. The AHR pathway has been recognized for its role in the pathogenesis of diseases and disorders including cancer and viral infections.

Thus, the disclosure provides a method of modulating the aryl hydrocarbon receptor (AHR) in a cell comprising contacting the cell with a therapeutically effective amount of a compound or salt disclosed herein (such as a compound of Formula (I), Table 1, and salts thereof), or a formulation thereof, in an amount effective to modulate the AHR. In some cases, the contacting occurs in vitro. In some cases, the contacting occurs in vivo.

In some cases, the contacting comprises administering to a subject in need thereof. As used herein, the terms “patient” and “subject” may be used interchangeably and mean animals, such as dogs, cats, cows, horses, and sheep (i.e., non-human animals) and humans. Particular patients are mammals (e.g., humans). In some cases, the subject suffers from a disease or disorder, e.g., cancer or a viral infection.

Another aspect of the disclosure provides a method of treating a disease or disorder in a subject, comprising administering to the subject a therapeutically effective amount of a compound or salt disclosed herein (such as a compound of Formula (I), Table 1, and salts thereof), or a composition thereof. In some cases, the terms “treating”, “treat” or “treatment” and the like can include preventative (e.g., prophylactic) and palliative treatment. In some cases, the disease or disorder is cancer or a viral infection.

In some cases, the disease or disorder is cancer. In some cases, a cancer to be treated using the compounds and methods described herein include, but are not limited to, a hematological cancer, a lymphoma, a myeloma, a leukemia, a neurological cancer, skin cancer, breast cancer, a prostate cancer, a cancer of the respiratory tract, a cancer of the reproductive organs, a cancer of the digestive tract, a colorectal cancer, lung cancer, head and neck cancer, a gastrointestinal cancer, a liver cancer, a pancreatic cancer, a genitourinary cancer, a bone cancer, renal cancer, and a vascular cancer. In some cases, the cancer is cancer is a liquid or solid tumor, hematological cancer, lymphoma, myeloma, leukemia, sarcoma, eye cancer, thyroid cancer, parathyroid cancer, neurological cancer, skin cancer, breast cancer, endocrine, uterine cancer, endometrial cancer, prostate cancer, colorectal cancer, lung cancer, head and neck cancer, gastrointestinal cancer, liver cancer, pancreatic cancer, genitourinary cancer, bone cancer, renal cancer, or vascular cancer. In some cases, the cancer is bone cancer. Contemplated bone cancers include, but are not limited to, Histiocytoma of Bone. In some cases, the cancer is endocrine cancer. Contemplated endocrine cancers include but are not limited to Multiple Endocrine Neoplasia Syndrome, adrenal cancer (such as without limitation adrenocortical cancer, adrenocortical carcinoma, or adrenocortical adenoma), thyroid cancer (such as without limitation anaplastic thyroid cancer), and parathyroid cancer. In some cases, the cancer is breast cancer. Contemplated breast cancers include, but are not limited to, triple negative breast cancer, invasive ductal carcinoma, invasive lobular carcinoma, ductal carcinoma in situ, and lobular carcinoma in situ. In some cases, the cancer is a cancer of the respiratory tract. Contemplated cancers of the respiratory tract include, but are not limited to, bronchogenic carcinoma, lung carcinoma (such as without limitation small-cell and non-small-cell lung carcinoma), as well as bronchial adenoma and pleuropulmonary blastoma. In some cases, the cancer is central nervous system cancer. Contemplated central nervous system cancers include, but are not limited to, spinal axis tumors, neurofibromatosis-1 associated malignant peripheral nerve sheath tumors (MPNST), and brain cancer. In some cases, the cancer is brain cancer. Contemplated brain cancers include, but are not limited to, glioma, low-grade glioma, astrocytoma (e.g. Grade I—Pilocytic Astrocytoma, Grade II—Low-grade Astrocytoma, Grade III—Anaplastic Astrocytoma, or Grade IV—Glioblastoma (GBM)), glioblastoma multiforme (GBM, also known as glioblastoma), medulloblastoma, craniopharyngioma, ependymoma, Pineal Parenchymal Tumors of Intermediate Differentiation, pinealoma, hemangioblastoma, acoustic neuroma, oligodendroglioma, meningioma, melanoma, neuroblastoma, Esthesioneuroblastoma, or retinoblastoma, medullary carcinoma, brain stem and hypophtalmic glioma, cerebellar and cerebral astrocytoma, as well as neuroectodermal and pineal tumor, chordoma, mixed glioma, optic nerve glioma, subependymoma, metastatic brain tumor, pituitary tumors, pituitary adenoma, primitive neuroectodermal (PNET) tumor, or schwannoma. In some cases, the cancer is a type found more commonly in children than adults, such as brain stem glioma, juvenile pilocytic astrocytoma, optic nerve glioma, pineal tumor, primitive neuroectodermal tumors (PNET), or rhabdoid tumor. In some cases, the patient is an adult human. In some cases, the patient is a child or pediatric patient. In some cases, the cancer is a cancer of the male reproductive organs. Contemplated cancers of the male reproductive organs include, but are not limited to, embryonal carcinoma, seminoma, penile cancer, prostate cancer, and testicular cancer. In some cases, the cancer is a cancer of the female reproductive system. Contemplated cancers of the female reproductive organs include, but are not limited to, ovarian epithelial cancer, fallopian tube cancer, uterine papillary serous carcinoma (UPSC), choriocarcinoma, endometrial, cervical, ovarian, vaginal, and vulvar cancer, as well as sarcoma of the uterus. In some cases, the cancer is an ovarian cancer. Contemplated ovarian cancers include, but are not limited to serous tumor, papillary serous cystadenocarcinoma, endometrioid tumor, mucinous cystadenocarcinoma, granulosa cell tumor, Sertoli-Leydig cell tumor and arrhenoblastoma. In some cases, the cancer is cervical cancer. Contemplated cervical cancers include, but are not limited to squamous cell carcinoma, adenocarcinoma, adenosquamous carcinoma, small cell carcinoma, neuroendocrine tumor, glassy cell carcinoma and villoglandular adenocarcinoma. In some cases, the cancer is a cancer of the digestive tract. Contemplated cancers of the digestive tract include, but are not limited to, anal, colon, colon carcinoma, colorectal, esophageal, stomach, gallbladder, gastrointestinal, Gastrointestinal Carcinoid Tumor, Gastrointestinal Stromal Tumors (GIST), duodenal, gastric, pancreatic, rectal, small-intestine, and salivary gland cancers. In some cases, the cancer is esophageal cancer. Contemplated esophageal cancer include, but are not limited to esophageal cell carcinomas and adenocarcinomas, as well as squamous cell carcinomas, leiomyosarcoma, malignant melanoma, rhabdomyosarcoma and lymphoma. In some cases, the cancer is gastric cancer. Contemplated gastric cancers include, but are not limited to intestinal type and diffuse type gastric adenocarcinoma. In some cases, the cancer is pancreatic cancer. Contemplated pancreatic cancers include, but are not limited to pancreatic ductal carcinoma, pancreatic adenocarcinoma, ductal adenocarcinoma, adenosquamous carcinomas and pancreatic endocrine tumors. In some cases, the cancer is cancer of the urinary tract. Contemplated cancers of the urinary tract include, but are not limited to, bladder, penile, kidney, renal pelvis, ureter, urethral and human papillary renal cancers. In some cases, the cancer is a kidney cancer. Contemplated kidney cancers include, but are not limited to renal cell carcinoma, carcinoma of the renal pelvis, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor. In some cases, the cancer is bladder cancer. Contemplated bladder cancers include, but are not limited to bladder carcinoma, transitional cell carcinoma, squamous cell carcinoma, adenocarcinoma, sarcoma and small cell carcinoma. In some cases, the cancer is eye cancer. Contemplated eye cancers include, but are not limited to, intraocular melanoma and retinoblastoma. In some cases, the cancer is liver cancer. Contemplated liver cancers include, but are not limited to, cystadenoma, hepatoma, bile duct carcinoma, Extrahepatic Bile Duct Cancer, hepatobilliary (hepatic and billiary duct) cancer, hepatoblastoma, hepatocellular carcinoma (liver cell carcinomas with or without fibrolamellar variant), cholangiocarcinoma (intrahepatic bile duct carcinoma), hepatocholangiocarcinoma and mixed hepatocellular cholangiocarcinoma. In some cases, the cancer is skin cancer. Contemplated skin cancers include, but are not limited to, basal cell carcinoma, sweat gland carcinoma, sebaceous gland carcinoma, squamous cell carcinoma, Kaposi's sarcoma, malignant melanoma, Merkel cell skin cancer, and non-melanoma skin cancer. In some cases, the cancer is head-and-neck cancer. Contemplated head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell. In some cases, the cancer is lymphoma. Contemplated lymphomas include, but are not limited to, lymphocytic lymphoma, AIDS-related lymphoma, non-Hodgkin's lymphoma, cutaneous T-cell lymphoma, Burkitt lymphoma, Hodgkin's disease, and lymphoma of the central nervous system. In some cases, the cancer is sarcoma. Contemplated sarcomas include, but are not limited to, sarcoma of the soft tissue, fibrosarcoma, neurofibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, malignant fibrous histiocytoma, lymphosarcoma, and rhabdomyosarcoma. In some cases, the cancer is leukemia. Contemplated leukemias include, but are not limited to, acute leukemia (such as without limitation acute myeloid leukemia, acute lymphocytic leukemia, acute lymphoblastic leukemia, chronic lymphocytic leukemia, acute myelocytic leukemia, acute myeloblastic leukemia, acute promyelocytic leukemia, acute myelomonocytic leukemia, acute monocytic leukemia, and acute erythroleukemia), chronic leukemia (such as without limitation chronic myelocytic leukemia and chronic myelogenous leukemia), and hairy cell leukemia.

Other cancers contemplated for treatment with the compounds of the disclosure (such as a compound of Formula (I), Table 1, and salts thereof) and methods described herein can be selected from, e.g., urothelial carcinomas, including, but not limited to, bladder cancer and all transitional cell carcinomas; head and neck squamous cell carcinoma; melanoma, including, but not limited to, uveal melanoma; ovarian cancer, including, but not limited to, a serous subtype of ovarian cancer; renal cell carcinoma, including, but not limited to, clear cell renal cell carcinoma subtype; cervical cancer; gastrointestinal/stomach (GIST) cancer, including but not limited to, stomach cancer; non-small cell lung cancer (NSCLC); acute myeloid leukemia (AML); and esophageal cancers. In some cases, the cancer is a urothelial carcinoma. In some cases, the cancer is bladder cancer. In some cases, the cancer is a transitional cell carcinoma. In some cases, the cancer is head and neck squamous cell carcinoma. In some cases, the cancer is a melanoma. In some cases, the cancer is a uveal melanoma. In some cases, the cancer is ovarian cancer. In some cases, the cancer is a serous subtype of ovarian cancer. In some cases, the cancer is renal cell carcinoma. In some cases, the cancer is a clear cell renal cell carcinoma subtype. In some cases, the cancer is cervical cancer. In some cases, the cancer is a gastrointestinal/stomach (GIST) cancer. In some cases, the cancer is a stomach cancer. In some cases, the cancer is non-small cell lung cancer (NSCLC). In some cases, the cancer is advanced and/or metastatic NSCLC. In some cases, the cancer is an esophageal cancer.

Other contemplated cancers include without limitation polycythemia vera, Waldenstrom's macroglobulinemia, multiple myeloma, heavy chain disease, epithelial carcinoma, Appendix Cancer, Atypical Teratoid/Rhabdoid Tumor, Central Nervous System Atypical Teratoid/Rhabdoid Tumor, Central Nervous System Embryonal Tumors, Bronchial Tumors, Carcinoid Tumor, Carcinoma of Unknown Primary, Central Nervous System Cancer, Childhood Cancers, Chordoma, Chronic Myeloproliferative Disorders, Ductal Carcinoma In Situ (DCIS), Embryonal Tumors, Endometrial Cancer, Ependymoblastoma, Extracranial Germ Cell Tumor, Extragonadal Germ Cell Tumor, Eye Cancer, FibrousGerm Cell Tumor, Gestational Trophoblastic Tumor, Heart Cancer, Histiocytosis, Langerhans Cell Cancer, Hypopharyngeal Cancer, Islet Cell Tumors, Langerhans Cell Histiocytosis, Laryngeal Cancer, Lip and Oral Cavity Cancer, Lobular Carcinoma In Situ (LCIS), Macroglobulinemia, Medulloepithelioma, Merkel Cell Carcinoma, Midline Tract Carcinoma Involving NUT Gene, Mouth Cancer, Multiple Endocrine Neoplasia Syndrome, Plasma Cell Neoplasm, Mycosis Fungoides, Myelodysplastic Syndrome, Myelodysplastic/Myeloproliferative Neoplasm, Myeloma, Chronic Myeloproliferative Disorder, Nasal Cavity Cancer, Paranasal Sinus Cancer, Nasopharyngeal Cancer, Oral Cancer, Oral Cavity Cancer, Lip Cancer, Oropharyngeal Cancer, Papillomatosis, Paraganglioma, Paranasal Sinus Cancer, Nasal Cavity Cancer, Pharyngeal Cancer, Pheochromocytoma, Pineoblastoma, Plasma Cell Neoplasm, Pleuropulmonary Blastoma, Transitional Cell Cancer, Sezary Syndrome, Supratentorial Primitive Neuroectodermal Tumors, Throat Cancer, Thymoma, Thymic Carcinoma, Transitional Cell Gestational Trophoblastic Tumor, Unknown Primary, Unusual Cancer of Childhood, or diffuse large B-cell lymphoma (DLBCL).

Also provided are methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-1 in leukemic cells; as well as virus-associated tumors in gastric cancer, nasopharyngeal carcinoma, cervical cancer, vaginal cancer, vulvar cancer, squamous cell carcinoma of the head and neck, and Merkel cell carcinoma.

In some cases, the compounds of the disclosure (such as a compound of Formula (I), Table 1, and salts thereof), or a composition thereof are useful for the treatment of a viral infection. In some cases, the viral infection to be treated using the compounds and methods described herein include, but are not limited to, coronavirus infections and flavivirus infections.

In some cases, the viral infection is a coronavirus infection. The term “coronavirus infection” as used herein means a disease caused by an infection with a coronavirus. Coronaviruses are a family of viruses that cause diseases in mammals and birds. Coronaviruses are in the subfamily Orthocoronavirinae in the family Coronaviridae, in the order Nidovirales. There are four main genera of coronaviruses, known as alpha, beta, gamma, and delta. Coronaviruses that affect humans include Human coronavirus 229E (HCoV-229E), Human coronavirus OC43 (HCoV—OC43), Severe acute respiratory syndrome-related coronavirus (SARS-CoV), Human coronavirus NL63 (HCoV-NL63, New Haven coronavirus), Human coronavirus HKU1, Middle East respiratory syndrome-related coronavirus (MERS-CoV, previously known as novel coronavirus 2012 and HCoV-EMC), and SARS-CoV-2 (also known as 2019-nCoV).

In humans, coronaviruses cause respiratory infections, including the common cold, which are typically mild, though rarer forms such as SARS, MERS and SARS-CoV-2 (the cause of the 2019-20 COVID-19 outbreak) can be lethal. Symptoms vary in other species: in chickens, they cause an upper respiratory disease, while in cows and pigs coronaviruses cause diarrhea. There are no vaccines or antiviral drugs to prevent or treat human coronavirus infections. The coronaviruses HCoV-229E, -NL63, —OC43, and —HKU1 continually circulate in the human population and cause respiratory infections in adults and children worldwide. Non-limiting examples of coronaviruses include severe acute respiratory syndrome-related coronavirus (SARS), Middle East respiratory syndrome-related coronavirus (MERS), and SARS-CoV-2 virus (also known as 2019-nCoV). Thus, in some cases, the compounds of the disclosure (such as a compound of Formula (I), a compound of Table 1, and salts thereof), or pharmaceutically acceptable salt thereof can be used to treat SARS, MERS, and COVID-19 (i.e., SARS-CoV infection, MERS-CoV infection, and SARS-CoV-2 infection, respectively).

In some cases, the viral infection is a flavivirus infection. The term “flavivirus infection” as used herein means the disease caused by an infection with a flavivirus.

Flaviviruses are a family of viruses that cause diseases in mammals and insects. Flaviviruses are in the family Flaviviridae, in the order Amarillovirales. Flaviviruses have positive-sense, single-stranded RNA genomes which are non-segmented and around 10-11 kbp in length. In humans, flaviviruses cause hemorrhagic fever, encephalitis, and the birth defect microcephaly. Flavivirus infections range from being asymptomatic to causing death. There are vaccines or antiviral drugs to prevent or treat some, but not all, human flavivirus infections. Non-limiting examples of flaviviruses include West Nile virus, dengue virus, tick-borne encephalitis virus, yellow fever virus, Zika virus and several other viruses which may cause encephalitis. Thus, in some cases, the compounds of the disclosure (such as a compound of Formula (I), a compound of Table 1, and salts thereof), or pharmaceutically acceptable salt thereof can be used to treat flavivirus infections, such as West Nile fever, dengue fever, tick-borne encephalitis, yellow fever, and Zika fever (i.e., West Nile virus infection, dengue virus infection, tick-borne encephalitis virus infection, yellow fever virus infection, and Zika virus infection, respectively).

Another aspect of the disclosure provides the use of a compound or salt disclosed herein (such as a compound of Formula (I), a compound of Table 1, and salts thereof), or a composition comprising a compound or salt disclosed herein (such as a compound of Formula (I), a compound of Table 1, and salts thereof) in the treatment of a disease or disorder (e.g., cancer or a viral infection).

Also contemplated is the use of a compound or salt disclosed herein (such as a compound of Formula (I), a compound of Table 1, and salts thereof), or a composition comprising a compound or salt disclosed herein (such as a compound of Formula (I), a compound of Table 1, and salts thereof) in the manufacture of a medicament in the treatment of a disease or disorder (e.g., cancer or a viral infection).

Use of a compound as disclosed herein (such as a compound of Formula (I), a compound of Table 1, and salts thereof), or a pharmaceutically acceptable salt thereof to treat a disease or disorder (e.g., cancer or a viral infection) in a subject also is contemplated. Further, the use of a compound as disclosed herein (such as a compound of Formula (I), a compound of Table 1, and salts thereof), or a pharmaceutically acceptable salt thereof, also is contemplated. Furthermore, use of a compound as disclosed herein (such as a compound of Formula (I), a compound of Table 1, and salts thereof), or a pharmaceutically acceptable salt in the preparation of a medicament for use in treating the aforementioned conditions also are contemplated.

In some cases, the compound as disclosed herein (such as a compound of Formula (I), a compound of Table 1, and salts thereof), or pharmaceutically acceptable salt thereof, can be administered in combination with another therapeutic agent to treat a disease or disorder, (e.g., cancer or a viral infection). Thus, in any of the methods disclosed herein, treatment of a disease or disorder (e.g., cancer or a viral infection) includes co-administration of the compound of Formula (I), Table 1, or a pharmaceutically acceptable salt thereof in combination with another therapeutic agent. In some cases, the therapeutic agent is an immune checkpoint inhibitor. Non-limiting examples of immune checkpoint inhibitors include PD1 or PDL1 antibodies, such as pembrolizumab, nivolumad, cemiplimab, atezolizumab, dostarlimab, durvalumab, or avelumab.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Synthesis of the Compounds of the Disclosure

The compounds of the disclosure can be synthesized by any method known in the art. For example, the compounds of Formula (I) or Table 1, and salts thereof, can be synthesized according to Scheme 1 or Scheme 2.

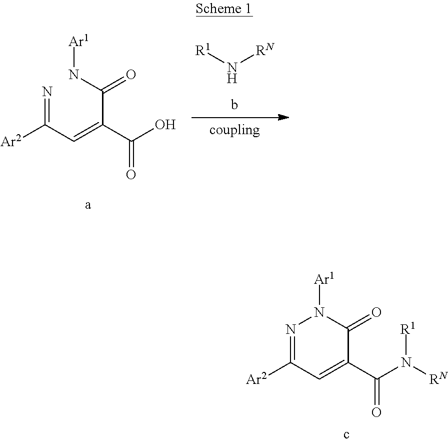

In some cases, compounds of Formula (I) having structure c can be synthesized using the procedure shown in Scheme 1. Coupling of a substituted 3-oxo-2,3-dihydropyridazine-4-carboxylic acid a with an amine compound b produces substituted 3-oxo-2,3-dihydropyridazine-4-amide compounds having structure c.

Alternatively, compound c can also be made by connecting an Ar² group to the scaffold as the last step via cross coupling reactions, such as the Suzuki coupling, as shown in Scheme 2.

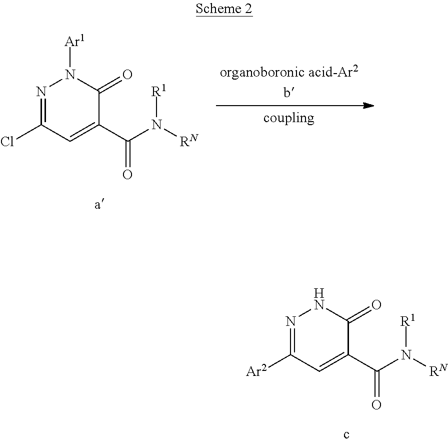

Optional further derivatization steps produce compounds as described herein, i.e., compounds of Formula I having structure c or a modified structure c.

The coupling of compounds a and b and a′ and b′ can be catalyzed or facilitated by appropriate reagents selected based on the precise nature of compounds a and b and a′ and b′. For example, the coupling of carboxylic acid compound a and amine compound b can be carried out using carbodiimide chemistry e.g., by using CDI or DIEA/HATU in an appropriate solvent, e.g., DMF.

Compounds a and b and a′ and b′ can be purchased commercially or prepared by a variety of methods from commercially-available starting materials. Optional derivatization reactions to transform compounds having structure c into compounds having a modified structure c can be selected based on the nature of the substituent R¹ in compound c, and the functionality desired in the modified structure c. For example, when compound c has a primary amine group on R¹, the terminal amine functionality can be further derivatized by methods known in the art to form a variety of functional groups. For example, the amine moiety can be acylated with e.g., an acid chloride or other appropriate reagent.

Also disclosed herein are compounds useful as synthetic intermediates in the synthesis of the AHR modulators disclosed herein. For example, provided herein are compounds of formula (A) or (B):

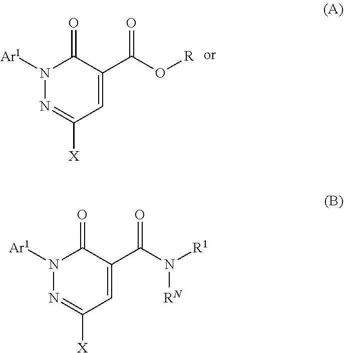

wherein Ar¹, R³, R¹ and R^{N} are as defined above, X is a leaving group, and R is H, C₁₋₆alkyl, or phenyl. As used herein, a leaving group is a functional group that is compatible with aryl coupling, e.g., a Suzuki coupling, such as halo (e.g., chloro), alkylsulfonyloxy or arylsulfonyloxy (e.g., methanesulfonyloxy, ethanesulfonyloxy, benzenesulfonyloxy, tosyloxy, triflyl). In some cases, X is chloro, fluoro, mesyl, tosyl, or triflyl. Compounds of Formula A or B can react with an Ar² precursor under suitable coupling conditions (e.g., in the presence of a coupling catalyst), to couple Ar² to the Compound of Formula A or B, at the “X” position, and thereby form the compound of Formula I. A coupling catalyst can be, e.g., a palladium catalyst (e.g., chloro(crotyl)(tri-tert-butylphosphine)palladium(II), or Pd(dppf)Cl₂). The Ar² precursor can be a Ar² boronic acid or boronic ester.

In some cases, Compound (A) or (B) can be reacted with an amine nucleophile which can then be reacted to form the Ar² moiety, and further modified as necessary (e.g., to insert the —NR^{N}R¹ moiety), to form the compound of Formula (I).

### EXAMPLES

The following examples are provided for illustration and are not intended to limit the scope of the invention.

### Example 1: Analog Preparation Scheme A

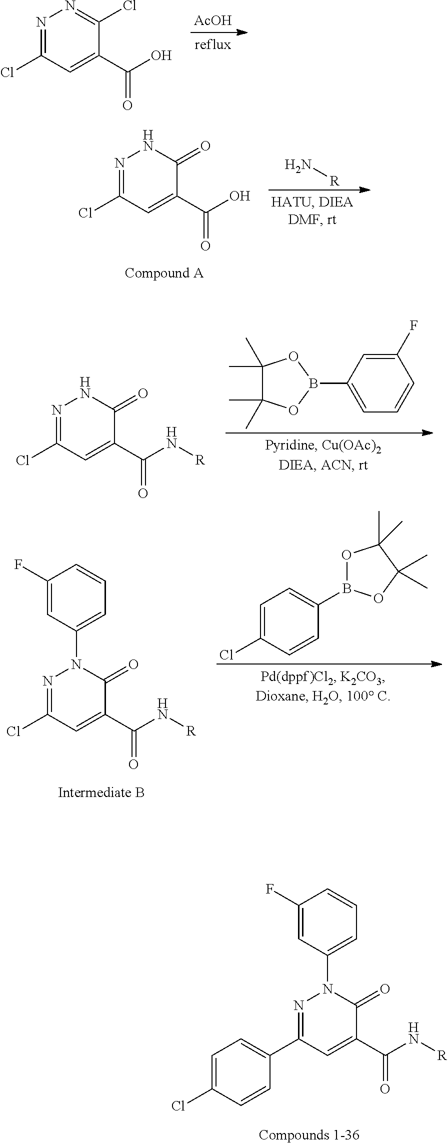

A solution of 3,6-dichloropyridazine-4-carboxylic acid (15.0 g, 0.078 mol) in AcOH (150 ml) was heated to 120° C. and stirred for 4 hours. After cooling, the reaction mixture was poured into water (300 ml) and a solid was precipitated, filtered, washed with water, and dried to afford compound A. LCMS (ESI) calcd for C₅H₃ClN₂O₃ [M+H]⁺ ms/z=175.1, found 175.1.

To a solution of 6-chloro-3-oxo-2,3-dihydropyridazine-4-carboxylic acid (174 mg, 1.0 mmol) in DMF (1.0 ml) was added DIEA (387 mg, 3.0 mmol) and HATU (760 g, 2.0 mmol). After 10 min, the corresponding amine (1.2 mmol) was added. The reaction mixture was stirred at 25° C. for 2 h. The LCMS showed the desired MS was detected. The solvent was removed under vacuum and the residue was extracted with EtOAc (20 mL). The organic layer was washed with brine, dried over Na₂SO₄, filtered and concentrated to yield the desired product which was purified by flash chromatography to yield the corresponding amide.

A solution of obtained amide compound (1.0 mmol), 2-(3-fluorophenyl)-4,4,5,5-tetramethyl-1,3,2-dioxaborolane (266.0 mg, 1.2 mmol), DIEA (154.8 mg, 1.2 mmol), pyridine (2.0 mmol) and Cu(OAc)₂ (218 mg, 1.3 mmol) in ACN (2.0 ml) was stirred for 16 h at 25° C. The solvent was removed under vacuum and the residue was extracted with EtOAc (20 mL). The organic layer was washed with brine, dried over Na₂SO₄, filtered and concentrated to yield the desired product, which was then purified by flash chromatography (eluting with DCM/MeOH from 100:0 to 90:10 in 30 min) to afford intermediate B.

To a solution of intermediate B (0.2 mmol) in 1,4-dioxane/H₂O (2 mL/0.2 mL) was added 2-(4-chlorophenyl)-4,4,5,5-tetramethyl-1,3,2-dioxaborolane (0.24 mmol), Pd(dppf)Cl₂ (0.02 mmol) and K₂CO₃ (0.4 mmol). The reaction mixture was stirred at 100° C. for 16 h under nitrogen. The LCMS showed the desired MS was detected. The solvent was removed under vacuum. The residue was purified by flash chromatography (eluting with DCM/MeOH from 100:0 to 90:10 in 30 min) to afford the desired product, which was purified by prep-HPLC (columns: Gemini 5 μm C18 150×21.2 mm, mobile phase: MeCN—H₂O (0.1% FA), gradient: 50-95, 6.5 min) to afford final analogs.

### Example 2. Analog Preparation Scheme B

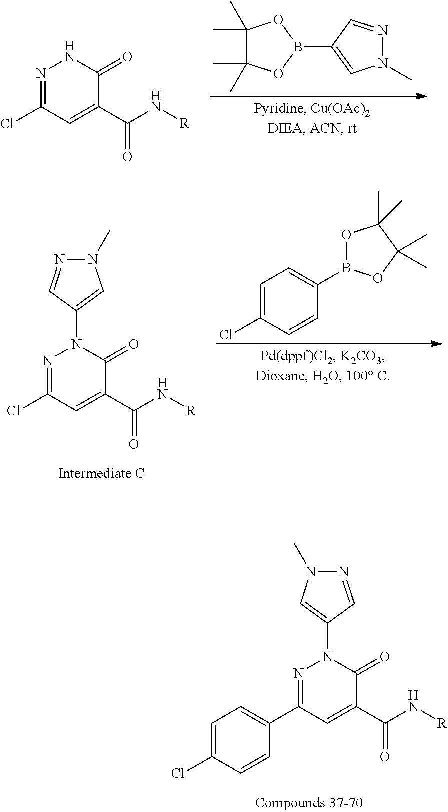

A solution of corresponding amide (1.0 mmol), 1-methyl-4-(4,4,5,5-tetramethyl-1,3,2-dioxaborolan-2-yl) pyrazole (1.2 mmol), DIEA (154.8 mg, 1.2 mmol), pyridine (2.0 mmol) and Cu(OAc)₂ (218 mg, 1.3 mmol) in ACN (2.0 ml) was stirred for 16 h at 25° C. The solvent was removed under vacuum and the residue was extracted with EtOAc (20 mL). The organic layer was washed with brine, dried over Na₂SO₄, filtered and concentrated to get the desired product which was purified by flash chromatography (eluting with DCM/MeOH from 100:0 to 90:10 in 30 min) to afford intermediate C.

To a solution of intermediate C (0.2 mmol) in 1,4-dioxane/H₂O (2 mL/0.2 mL) was added 2-(4-chlorophenyl)-4,4,5,5-tetramethyl-1,3,2-dioxaborolane (0.24 mmol), Pd(dppf)Cl₂ (0.02 mmol) and K₂CO₃ (0.4 mmol). The reaction mixture was stirred at 100° C. for 16 h under nitrogen. The LCMS showed the desired MS was detected. The solvent was removed under vacuum. The residue was purified by flash chromatography (eluting with DCM/MeOH from 100:0 to 90:10 in 30 min) to afford the desired product, which was then purified by prep-HPLC (columns: Gemini 5 μm C18 150×21.2 mm, mobile phase: MeCN—H₂O (0.1% FA), gradient: 50-95, 6.5 min) to afford final analogs.

### Example 3. Analog Preparation Scheme C

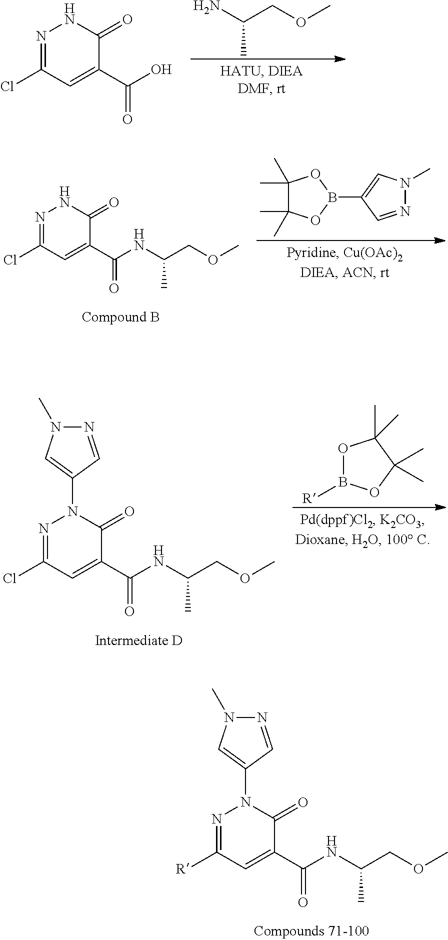

To a solution of 6-chloro-3-oxo-2,3-dihydropyridazine-4-carboxylic acid (3.0 g, 0.017 mol) in DMF (20 ml) was added DIEA (6.66 g, 0.052 mol) and HATU (13.1 g, 0.034 mol). After 10 min, (2S)-1-methoxypropan-2-amine (1.84 g, 0.021 mol) was added. The reaction mixture was stirred at 25° C. for 2 h. The solvent was removed under vacuum and the residue was extracted with EtOAc (20 mL). The organic layer was washed with brine, dried over Na₂SO₄, filtered, and concentrated to yield the desired product which was purified by flash chromatography (eluting with PE/EtOAc from 100:0 to 50:50 in 30 min) to afford compound B. LCMS (ESI) calcd for C₉H₁₂ClN₃O₃ [M+H]⁺ ms/z=246.2, found 246.2.

A solution of compound B (300.0 mg, 1.22 mmol), 1-methyl-4-(4,4,5,5-tetramethyl-1,3,2-dioxaborolan-2-yl) pyrazole (1.47 mmol), DIEA (189.4 mg, 1.47 mmol), pyridine (193.2 mg, 2.44 mmol) and Cu(OAc)₂ (277.3 g, 1.53 mmol) in ACN (10 ml) was stirred for 16 h at 25° C., The LCMS showed the desired MS was detected. The solvent was removed under vacuum and the residue was extracted with EtOAc (20 mL). The organic layer was washed with brine, dried over Na₂SO₄, filtered, and concentrated to yield the desired product which was purified by flash chromatography (eluting with DCM/MeOH from 100:0 to 90:10 in 30 min) to afford intermediate D. LCMS (ESI) calcd for C₁₃H₁₆ClN₅O₃ [M+H]⁺ ms/z=326.2, found 326.2.

To a solution of intermediate D (0.2 mmol) in 1,4-dioxane/H₂O (2 mL/0.2 mL) was added the corresponding borate (0.24 mmol), Pd(dppf)Cl₂ (0.02 mmol) and K₂CO₃ (0.4 mmol). The reaction mixture was stirred at 100° C. for 16 h under nitrogen. The LCMS showed the desired MS was detected. The solvent was removed under vacuum. The residue was purified by flash chromatography (eluting with DCM/MeOH from 100:0 to 90:10 in 30 min) to afford the desired product, which was purified by prep-HPLC (columns: Gemini 5 μm C18 150×21.2 mm, mobile phase: MeCN—H₂O (0.1% FA), gradient: 50-95, 6.5 min) to afford analogs.

### Example 4. Preparation of Compound 57

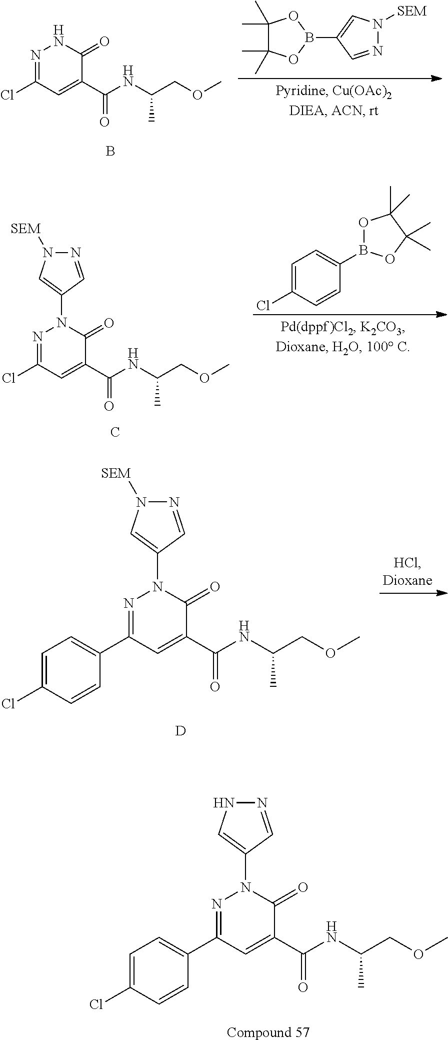

A solution of compound B (300.0 mg, 1.22 mmol), 4-(4,4,5,5-tetramethyl-1,3,2-dioxaborolan-2-yl)-1-([2-(trimethylsilyl)ethoxy]methyl)-1H-pyrazole (476.7 mg, 1.47 mmol), DIEA (189.4 mg, 1.47 mmol), pyridine (193.2 mg, 2.44 mmol) and Cu(OAc)₂ (277.3 g, 1.53 mmol) in ACN (10 ml) was stirred for 16 h at 25° C. The solvent was removed under vacuum and the residue was extracted with EtOAc (20 mL). The organic layer was washed with brine, dried over Na₂SO₄, filtered and concentrated to yield the desired product which was purified by flash chromatography (eluting with DCM/MeOH from 100:0 to 90:10 in 30 min) to afford compound C.

To a solution of compound C (53.0 mg, 0.2 mmol) in 1,4-dioxane/H₂O (2 mL/0.2 mL) was added 2-(4-chlorophenyl)-4,4,5,5-tetramethyl-1,3,2-dioxaborolane (57.2 mg, 0.24 mmol), Pd(dppf)Cl₂ (0.02 mmol) and K₂CO₃ (0.4 mmol). The reaction mixture was stirred at 100° C. for 16 h under nitrogen. The solvent was removed under vacuum. The residue was purified by flash chromatography (eluting with DCM/MeOH from 100:0 to 90:10 in 30 min) to yield the desired product was purified by prep-HPLC (columns: Gemini 5 μm C18 150×21.2 mm, mobile phase: MeCN—H₂O (0.1% FA), gradient: 50-95, 6.5 min) to afford compound D. LCMS (ESI) calcd for C₂₄H₃₂ClN₅O₄Si [M+H]⁺ ms/z=518.2, found 518.2.

A solution of compound D (40.0 mg, 0.093 mmol) in HCl-dioxane was stirred for 120 h at 25° C. The solvent was removed under vacuum to yield the desired product which was purified by prep-HPLC (columns: Gemini 5 μm C18 150×21.2 mm, mobile phase: ACN—H₂O (0.1% NH₃·H₂O), gradient: 50-95, 11.25 min) to afford compound 101. LCMS (ESI) calcd for C₁₈H₁₈ClN₅O₃ [M+H]⁺ ms/z=388.0, found 388.0.

### Example 5—Additional Synthesis Routes

### Method 1—Compounds 134, 135, 136, 137, 138, 142, 143, 151, 158, 159, 160, 161, 162, 188, 189, and 190

To a solution of 6-chloro-3-oxopyridazine-4-carboxamide derivatives (0.092 mmol), corresponding diazole or triazole or indole derivatives (0.111 mmol), cesium carbonate (60.0 mg, 0.1842 mmol) and catalytic agent [Copper(I) iodide (17.5 mg, 0.092 mmol) or Pd-PEPPSI-IPentCl₂-MePy (3.7 mg, 0.0046 mmol)] in dry DMF (3 ml). The reaction mixture was stirred at 100° C. for 1-16 h. The LCMS showed the desired MS was detected. The reaction mixture was concentrated. The crude product was purified by prep-TLC (PE/EtOAc=1/1) to get crude product which was purified by prep-HPLC (columns: Gemini 5 um C18 150×21.2 mm, mobile phase: ACN—H₂O (0.1% FA), gradient: 30-95, 11.25 min) to afford desired product.

### Method 2—Compounds 110, 114, 116, 117, 125, 127, 145

To a solution of 6-chloro-N-[(2S)-1-methoxypropan-2-yl]-2-(1-methylpyrazol-4-yl)-3-oxopyridazine-4-carboxamide (30.0 mg, 0.0880 mmol) in MeCN (3 mL) was added corresponding secondary amine (0.176 mmol) and potassium carbonate (24.3 mg, 0.176 mmol) stirred for 12 h at 100° C. The LCMS showed the desired MS was detected. The reaction mixture was concentrated. The residue was purified by prep-HPLC (columns: Gemini 5 um C18 150×21.2 mm, mobile phase: ACN—H₂O (0.1% FA), gradient: 30-95, 10.25 min) to afford desired product.

### Method 3—Compounds 115, 119, 149, 152

To a solution of 6-chloro-N-[(2S)-1-methoxypropan-2-yl]-2-(1-methylpyrazol-4-yl)-3-oxopyridazine-4-carboxamide (50.0 mg, 0.1535 mmol) in 1,4-dioxane/H₂O=5:1 (3 mL) was added borate derivatives (0.1842 mmol), Pd(dppf)Cl₂ (11.2 mg, 0.01535 mmol) and K₂CO₃ (42.4 mg, 0.307 mmol) stirred for 12 h at 100° C. The LCMS showed the desired MS was detected. The reaction mixture was concentrated. The reaction mixture was filtered out and concentrated. The residue was purified by prep-HPLC (columns: Gemini 5 um C18 150×21.2 mm, mobile phase: ACN—H₂O (0.1% FA), gradient: 40-95, 11.00 min) to afford desired product.

Mass spectrometry data for the compounds synthesized herein are presented in Table 2, below. Table 3 shows NMR and mass spectrometry data for compounds synthesized via the above described methods.

TABLE 2

| Cpd No. | LCMS (ESI): [M + H] ^{+.3s7y} |
|---|---|
| 1 | 402.1 |
| 2 | 429.1 |
| 3 | 457.1 |
| 4 | 344.0 |
| 5 | 399.1 |
| 6 | 402.1 |
| 7 | 416.1 |
| 8 | 401.1 |
| 9 | 415.1 |
| 10 | 416.1 |
| 11 | 415.1 |
| 12 | 429.1 |
| 13 | 443.1 |
| 14 | 401.1 |
| 15 | 415.1 |
| 16 | 429.1 |
| 17 | 387.1 |
| 18 | 401.1 |
| 19 | 415.1 |
| 20 | 443.1 |
| 21 | 358.0 |
| 22 | 402.1 |
| 23 | 401.1 |
| 24 | 400.0 |
| 25 | 413.1 |
| 26 | 427.1 |
| 27 | 441.1 |
| 28 | 455.1 |
| 29 | 456.1 |
| 30 | 441.1 |
| 31 | 416.1 |
| 32 | 402.1 |
| 33 | 388.1 |
| 34 | 387.1 |
| 35 | 401.1 |
| 36 | 415.1 |
| 37 | 402.1 |
| 38 | 388.1 |
| 39 | 387.1 |
| 40 | 401.1 |
| 41 | 415.1 |
| 42 | 402.1 |
| 43 | 401.1 |
| 44 | 415.1 |
| 45 | 429.1 |
| 46 | 387.1 |
| 47 | 401.1 |
| 48 | 415.1 |
| 49 | 443.1 |
| 50 | 373.1 |
| 51 | 387.1 |
| 52 | 401.1 |
| 53 | 429.1 |
| 54 | 330.0 |
| 55 | 344.0 |
| 56 | 388.0 |
| 57 | 387.1 |
| 58 | 386.1 |
| 59 | 385.1 |
| 60 | 399.1 |
| 61 | 427.1 |
| 62 | 388.1 |
| 63 | 427.1 |
| 64 | 441.1 |
| 65 | 442.1 |
| 66 | 446.1 |
| 67 | 383.2 |
| 68 | 382.1 |
| 69 | 452.1− |
| 70 | 436.2 |
| 71 | 402.1 |
| 72 | 368.2 |
| 73 | 386.1 |
| 74 | 398.2 |
| 75 | 412.2 |
| 76 | 382.1 |
| 77 | 415.1 |
| 78 | 452.1− |
| 79 | 389.1 |
| 80 | 443.1 |
| 81 | 396.2 |
| 82 | 398.1 |
| 83 | 393.2 |
| 85 | 420.1 |
| 87 | 417.1 |
| 88 | 417.1 |
| 89 | 413.2 |
| 90 | 439.2 |
| 91 | 385.2 |
| 92 | 441.1 |
| 93 | 455.1 |
| 94 | 443.2 |
| 95 | 429.1 |
| 96 | 443.1 |
| 97 | 416.1 |
| 98 | 429.1 |
| 99 | 443.1 |
| 100 | 415.1 |
| 101 | 388.0 |

TABLE 3

| | LCMS | |
|---|---|---|
| Cpd | (ESI): | |
| No. | [M + H] ⁺ | ¹H NMR |
| 102 | 420.1 | ¹H NMR (400 MHz, DMSO-d₆, ppm) 9.50 (d, J = 8.0 Hz, 1H), 8.60 (s, 1H), |
| | | 8.56 (s, 1H), 8.29 (dd, J = 7.2, 2.0 Hz, 1H), 8.14 (s, 1H), 8.11-8.08 (m, 1H), |
| | | 7.57 (t, J = 8.8 Hz, 1H), 4.25-4.19 (m, 1H), 3.93 (s, 3H), 3.46-3.38 (m, 2H), |
| | | 3.31 (s, 3H), 1.20 (d, J = 6.7 Hz, 3H). |
| 103 | 404.1 | ¹H NMR (400 MHz, CDCl₃, ppm) 9.68 (d, J = 7.2 Hz, 1H), 8.67 (s, 1H), 8.37 |
| | | (s, 1H), 8.14 (s, 1H), 7.82-7.77 (m, 1H), 7.67-7.65 (m, 1H), 7.34-7.26 (m, |
| | | 1H), 4.44-4.37 (m, 1H), 4.00 (s, 3H), 3.49 (d, J = 5.2 Hz, 2H), 3.42 (s, 3H), |
| | | 1.32 (d, J = 6.8 Hz, 3H). |
| 104 | 402.1 | ¹H NMR (400 MHz, DMSO-d₆, ppm) 9.50 (d, J = 8.0 Hz, 1H), 8.59 (s, 1H), |
| | | 8.56 (s, 1H), 8.13 (s, 1H), 8.09 (d, J = 2.0 Hz, 1H), 8.04-7.99 (m, 1H), 7.61- |
| | | 7.55 (m, 2H), 4.27-4.17 (m, 1H), 3.93 (s, 3H), 3.46-3.38 (m, 2H), 3.31 (s, |
| | | 3H), 1.20 (d, J = 6.8 Hz, 3H). |
| 105 | 386.1 | ¹H NMR (400 MHz, DMSO-d₆, ppm) 9.51 (d, J = 8.0 Hz, 1H), 8.58 (d, J = |
| | | 7.2 Hz, 2H), 8.14 (s, 1H), 7.93-7.90 (m, 2H), 7.62-7.56 (m, 1H), 7.40-7.34 |
| | | (m, 1H), 4.27-4.17 (m, 1H), 3.93 (s, 3H), 3.49-3.38 (m, 2H), 3.31 (s, 3H), |
| | | 1.21 (d, J = 6.8 Hz, 3H). |
| 106 | 448.1 | ¹H NMR (400 MHz, DMSO-d₆, ppm) 9.55 (d, J = 8.0 Hz, 1H), 8.62 (s, 1H), |
| | | 8.57 (s, 1H), 8.23-8.05 (t, J = 6.8 Hz, 3H), 7.85 (d, J = 8.4 Hz, 2H), 7.79 (d, |
| | | J = 8.4 Hz, 2H), 7.56 (d, J = 8.4 Hz, 2H), 4.27-4.18 (m, 1H), 3.94 (s, 3H), |
| | | 3.47-3.39 (m, 2H), 3.32 (s, 3H), 1.21 (d, J = 6.8 Hz, 3H). |
| 107 | 420.1 | ¹H NMR (400 MHz, DMSO-d₆, ppm) 9.50 (d, J = 8.0 Hz, 1H), 8.48 (s, 1H), |
| | | 8.32 (s, 1H), 7.97 (s, 1H), 7.79 (dd, J = 8.8, 6.4 Hz, 1H), 7.68 (dd, J = 8.8, |
| | | 2.4 Hz, 1H), 7.45-7.40 (m, 1H), 4.25-4.15 (m, 1H), 3.91 (s, 3H), 3.45-3.38 |
| | | (m, 2H), 3.31 (s, 3H), 1.19 (d, J = 6.8 Hz, 3H). |
| 108 | 386.1 | ¹H NMR (400 MHz, DMSO-d₆, ppm) 9.52 (d, J = 8.0 Hz, 1H), 8.51 (s, 1H), |
| | | 8.43 (s, 1H), 8.03 (s, 1H), 7.92 (t, J = 7.8 Hz, 1H), 7.61-7.56 (m, 1H), 7.44- |
| | | 7.37 (m, 2H), 4.23-4.17 (m, 1H), 3.92 (s, 3H), 3.45-3.38 (m, 2H), 3.32 (s, |
| | | 3H), 1.20 (d, J = 6.8 Hz, 3H). |
| 109 | 404.2 | ¹H NMR (400 MHz, DMSO-d₆, ppm) 9.52 (d, J = 8.0 Hz, 1H), 8.52 (s, 1H), |
| | | 8.41 (d, J = 2.0 Hz, 1H), 8.04 (s, 1H), 8.02-7.97 (m, 1H), 7.53-7.47 (m, 1H), |
| | | 7.31-7.26 (m, 1H), 4.25-4.16 (m, 1H), 3.92 (s, 3H), 3.45-3.38 (m, 2H), 3.31 |
| | | (s, 3H), 1.20 (d, J = 6.8 Hz, 3H). |
| 110 | 401.2 | ¹H NMR (400 MHz, DMSO-d₆, ppm) 9.82 (d, J = 8.0 Hz, 1H), 8.36 (s, 1H), |
| | | 7.89 (s, 1H), 7.61 (s, 1H), 4.22-4.12 (m, 1H), 3.88 (d, J = 1.6 Hz, 7H), 3.45- |
| | | 3.35 (m, 2H), 3.29 (s, 3H), 1.86-1.76 (m, 4H), 1.63-1.55 (m, 4H), 1.17 (d, J = |
| | | 6.8 Hz, 3H). |
| 111 | 424.2 | ¹H NMR (400 MHz, DMSO-d₆, ppm) 9.58 (d, J = 8.0 Hz, 1H), 8.52 (d, J = |
| | | 3.2 Hz, 2H), 8.10 (s, 1H), 7.78-7.73 (m, 2H), 6.90-6.84 (m, 1H), 4.25-4.16 |
| | | (m, 3H), 3.93 (s, 3H), 3.47-3.37 (m, 2H), 3.31 (s, 3H), 2.85 (t, J = 6.4 Hz, |
| | | 2H), 2.03-1.90 (m, 2H), 1.20 (d, J = 6.8 Hz, 3H). |
| 112 | 410.2 | ¹H NMR (400 MHz, DMSO-d₆, ppm) 9.59 (d, J = 8.0 Hz, 1H), 8.52 (d, J = |
| | | 8.0 Hz, 2H), 8.10 (s, 1H), 7.94 (s, 1H), 7.78 (d, J = 8.4 Hz, 1H), 6.89 (d, J = |
| | | 8.4 Hz, 1H), 4.62 (t, J = 8.8 Hz, 2H), 4.24-4.17 (m, 1H), 3.92 (s, 3H), 3.47- |
| | | 3.37 (m, 2H), 3.31 (s, 3H), 3.27 (t, J = 8.8 Hz, 2H), 1.20 (d, J = 6.8 Hz, 3H). |
| 113 | 448.2 | ¹H NMR (400 MHz, DMSO-d₆, ppm) 9.59 (d, J = 8.0 Hz, 1H), 8.75 (d, J = |
| | | 3.2 Hz, 1H), 8.59 (s, 2H), 8.22-8.16 (m, 2H), 8.04 (dd, J = 9.2, 2.0 Hz, 1H), |
| | | 7.95 (dd, J = 8.8,2.0 Hz, 1H), 7.41 (s, 1H), 7.28-7.20 (m, 1H), 4.29-4.20 (m, |
| | | 1H), 3.95 (s, 3H), 3.91 (s, 3H), 3.49-3.39 (m, 2H), 3.32 (s, 3H), 1.22 (d, J = |
| | | 6.8 Hz, 3H). |
| 114 | 389.2 | ¹H NMR (400 MHz, DMSO-d₆, ppm) 9.79 (d, J = 8.0 Hz, 1H), 8.38 (s, 1H), |
| | | 8.08 (s, 1H), 7.93 (s, 1H), 4.22-4.12 (m, 1H), 3.99 (d, J = 12.8 Hz, 2H), 3.88 |
| | | (s, 3H), 3.45-3.35 (m, 2H), 3.29 (s, 3H), 2.90-2.77 (m, 2H), 1.75-1.66 (m, |
| | | 2H), 1.63-1.53 (m, 1H), 1.27-1.18 (m, 2H), 1.17 (d, J = 6.8 Hz, 3H), 0.93 (d, |
| | | J = 6.4 Hz, 3H). |
| 115 | 374.2 | ¹H NMR (400 MHz, DMSO-d₆, ppm) 9.57 (d, J = 8.0 Hz, 1H), 8.42 (s, 1H), |
| | | 8.07 (s, 1H), 7.94 (s, 1H), 4.24-4.15 (m, 1H), 3.90 (s, 3H), 3.44-3.34 (m, |
| | | 2H), 3.29 (s, 3H), 2.80-2.70 (m, 1H), 1.91 (d, J = 12.8 Hz, 2H), 1.80 (d, J = |
| | | 12.4 Hz, 2H), 1.69 (d, J = 12.8 Hz, 1H), 1.53-1.32 (m, 4H), 1.30-1.22 (m, |
| | | 1H), 1.17 (d, J = 6.4 Hz, 3H). |
| 116 | 429.3 | ¹H NMR (400 MHz, DMSO-d₆, ppm) 9.80 (d, J = 8.0 Hz, 1H), 8.39 (s, 1H), |
| | | 8.09 (s, 1H), 7.94 (s, 1H), 4.22-4.12 (m, 1H), 3.88 (s, 3H), 3.44-3.36 (m, |
| | | 6H), 3.29 (s, 3H), 1.63-1.57 (m, 4H), 1.55-1.48 (m, 4H), 1.48-1.43 (m, 4H), |
| | | 1.17 (d, J = 6.8 Hz, 3H). |
| 117 | 415.2 | ¹H NMR (400 MHz, DMSO-d₆, ppm) 9.82 (d, J = 8.0 Hz, 1H), 8.36 (s, 1H), |
| | | 7.90 (s, 1H), 7.61 (s, 1H), 4.22-4.12 (m, 1H), 3.88 (s, 3H), 3.71 (s, 4H), |
| | | 3.43-3.34 (m, 2H), 3.29 (s, 3H), 1.70-1.60 (m, 4H), 1.49-1.31 (m, 6H), 1.17 |
| | | (d, J = 6.8 Hz, 3H). |
| 118 | 402.1 | ¹H NMR (400 MHz, DMSO-d₆, ppm) 9.51 (d, J = 8.0 Hz, 1H), 8.49 (s, 1H), |
| | | 8.33 (s, 1H), 7.98 (s, 1H), 7.74-7.71 (m, 1H), 7.66 (d, J = 7.6 Hz, 1H), 7.59- |
| | | 7.51 (m, 2H), 4.23-4.17 (m, 1H), 3.91 (s, 3H), 3.45-3.38 (m, 2H), 3.31 (s, |
| | | 3H), 1.20 (d, J = 6.8 Hz, 3H). |
| 119 | 402.2 | ¹H NMR (400 MHz, DMSO-d₆, ppm) 9.57 (d, J = 8.0 Hz, 1H), 8.43 (s, 1H), |
| | | 8.08 (s, 1H), 7.95 (s, 1H), 4.25-4.15 (m, 1H), 3.90 (s, 3H), 3.45-3.35 (m, |
| | | 2H), 3.29 (s, 3H), 2.73-2.62 (m, 1H), 1.82-1.73 (m, 2H), 1.70-1.57 (m, 2H), |
| | | 1.52-1.43 (m, 2H), 1.38-1.28 (m, 2H), 1.23 (s, 1H), 1.17 (d, J = 6.4 Hz, 3H), |
| | | 0.95 (s, 6H). |
| 120 | 436.2 | ¹H NMR (400 MHz, DMSO-d₆, ppm) 9.58 (d, J = 8.0 Hz, 1H), 8.79 (s, 1H), |
| | | 8.74 (s, 1H), 8.60 (s, 1H), 8.30 (d, J = 8.8 Hz, 1H), 8.24 (dd, J = 9.2, 6.0 Hz, |
| | | 1H), 8.20 (s, 1H), 8.07 (d, J = 8.8 Hz, 1H), 7.81 (dd, J = 10.4, 2.4 Hz, 1H), |
| | | 7.53 (d, J = 2.4 Hz, 1H), 4.31-4.19 (m, 1H), 3.95 (s, 3H), 3.49-3.39 (m, 2H), |
| | | 3.32 (s, 3H), 1.22 (d, J = 6.8 Hz, 3H). |
| 121 | 437.2 | ¹H NMR (400 MHz, DMSO-d₆, ppm) 9.55 (d, J = 8.0 Hz, 1H), 9.01 (dd, J = |
| | | 4.0, 1.6 Hz, 1H), 8.65 (d, J = 8.8 Hz, 1H), 8.61 (d, J = 8.4 Hz, 1H), 8.58-8.55 |
| | | (m, 2H), 8.13 (s, 1H), 7.98 (d, J = 12.8 Hz, 1H), 7.63 (dd, J = 8.4, 4.4 Hz, |
| | | 1H), 4.27-4.18 (m, 1H), 3.95 (s, 3H), 3.48-3.38 (m, 2H), 3.32 (s, 3H), 1.21 |
| | | (d, J = 6.4 Hz, 3H). |
| 122 | 449.2 | ¹H NMR (400 MHz, DMSO-d₆, ppm) 9.54 (d, J = 8.0 Hz, 1H), 8.71 (s, 1H), |
| | | 8.59 (s, 1H), 8.35-8.30 (m, 2H), 8.17-8.12 (m, 2H), 8.05 (d, J = 8.8 Hz, 1H), |
| | | 7.10 (d, J = 8.8 Hz, 1H), 4.30-4.20 (m, 1H), 4.04 (s, 3H), 3.95 (s, 3H), 3.48- |
| | | 3.38 (m, 2H), 3.32 (s, 3H), 1.22 (d, J = 6.8 Hz, 3H). |
| 123 | 436.2 | ¹H NMR (400 MHz, DMSO-d₆, ppm) 9.57 (d, J = 8.0 Hz, 1H), 8.79 (s, 1H), |
| | | 8.58 (s, 1H), 8.30 (s, 1H), 8.17 (s, 1H), 7.83 (s, 2H), 4.30-4.17 (m, 1H), 4.09 |
| | | (s, 3H), 3.95 (s, 3H), 3.48-3.38 (m, 2H), 3.32 (s, 6H), 1.22 (d, J = 6.8 Hz, |
| | | 3H). |
| 124 | 423.2 | ¹H NMR (400 MHz, DMSO-d₆, ppm) 9.56 (d, J = 8.0 Hz, 1H), 8.63 (s, 1H), |
| | | 8.58 (s, 1H), 8.32 (d, J = 1.6 Hz, 1H), 8.14 (s, 1H), 8.05 (dd, J = 8.8, 1.6 Hz, |
| | | 1H), 7.81 (d, J = 8.4 Hz, 1H), 4.30-4.17 (m, 1H), 3.94 (s, 3H), 3.48-3.38 (m, |
| | | 2H), 3.32 (s, 3H), 2.66 (s, 3H), 1.21 (d, J = 6.8 Hz, 3H). |
| 125 | 415.2 | ¹H NMR (400 MHz, DMSO-d₆, ppm) 9.92 (d, J = 8.0 Hz, 1H), 8.38 (s, 1H), |
| | | 7.95 (s, 1H), 7.79 (s, 1H), 4.22-4.10 (m, 1H), 3.88 (s, 3H), 3.52-3.43 (m, |
| | | 2H), 3.42-3.36 (m, 2H), 3.29 (s, 3H), 2.33 (s, 2H), 1.67-1.57 (m, 2H), 1.55- |
| | | 1.41 (m, 4H), 1.39-1.31 (m, 2H), 1.24-1.13 (m, 2H), 1.17 (d, J = 6.8 Hz, 3H). |
| 126 | 419.2 | ¹H NMR (400 MHz, DMSO-d₆, ppm) 9.57 (d, J = 8.0 Hz, 1H), 8.98 (dd, J = |
| | | 4.0, 1.6 Hz, 1H), 8.82 (s, 1H), 8.78 (d, J = 2.0 Hz, 1H), 8.61 (s, 1H), 8.59 (d, |
| | | J = 8.4 Hz, 1H), 8.51 (dd, J = 9.0, 2.0 Hz, 1H), 8.20 (s, 1H), 8.15 (d, J = 8.8 |
| | | Hz, 1H), 7.64 (dd, J = 8.4, 4.0 Hz, 1H), 4.31-4.19 (m, 1H), 3.95 (s, 3H), |
| | | 3.52-3.40 (m, 2H), 3.33 (s, 3H), 1.22 (d, J = 6.8 Hz, 3H). |
| 127 | 403.2 | ¹H NMR (400 MHz, DMSO-d₆, ppm) 9.80 (d, J = 7.6 Hz, 1H), 8.39 (s, 1H), |
| | | 8.09 (s, 1H), 7.94 (s, 1H), 4.17 (s, 1H), 3.88 (s, 3H), 3.45-3.35 (m, 6H), 3.29 |
| | | (s, 3H), 1.47-1.38 (m, 4H), 1.17 (d, J = 6.4 Hz, 3H), 0.97 (s, 6H). |
| 128 | 426.2 | ¹H NMR (400 MHz, DMSO-d₆, ppm) 9.55 (d, J = 8.0 Hz, 1H), 8.53 (s, 1H), |
| | | 8.49 (s, 1H), 8.08 (s, 1H), 7.52 (s, 1H), 7.50 (d, J = 2.0 Hz, 1H), 7.01 (d, J = |
| | | 8.8 Hz, 1H), 4.32 (s, 4H), 4.25-4.16 (m, 1H), 3.92 (s, 3H), 3.45-3.38 (m, |
| | | 2H), 3.31 (s, 3H), 1.20 (d, J = 6.8 Hz, 3H). |
| 129 | 433.2 | ¹H NMR (400 MHz, DMSO-d₆, ppm) 9.54 (d, J = 8.0 Hz, 1H), 8.85 (d, J = |
| | | 4.4 Hz, 1H), 8.75 (s, 1H), 8.60 (s, 1H), 8.57 (s, 1H), 8.36-8.28 (m, 8.8 Hz, |
| | | 1H), 8.25 (d, J = 8.8 Hz, 1H), 8.15 (s, 1H), 7.46 (d, J = 4.4 Hz, 1H), 4.30- |
| | | 4.20 (m, 1H), 3.95 (s, 3H), 3.49-3.39 (m, 2H), 3.32 (s, 3H), 2.74 (s, 3H), |
| | | 1.22 (d, J = 6.8 Hz, 3H). |
| 130 | 437.2 | ¹H NMR (400 MHz, DMSO-d₆, ppm) 9.54 (d, J = 8.0 Hz, 1H), 9.02 (d, J = |
| | | 3.6 Hz, 1H), 8.82 (s, 1H), 8.70-8.60 (m, 3H), 8.34 (d, J = 12.0 Hz, 1H), 8.25 |
| | | (s, 1H), 7.73 (dd, J = 8.0, 4.0 Hz, 1H), 4.25-4.20 (m, 1H), 3.95 (s, 3H), 3.50- |
| | | 3.40 (m, 2H), 3.32 (s, 3H), 1.22 (d, J = 6.4 Hz, 3H). |
| 131 | 434.2 | ¹H NMR (400 MHz, DMSO-d₆, ppm) 9.60 (d, J = 8.0 Hz, 1H), 9.00 (s, 1H), |
| | | 8.73 (s, 1H), 8.58 (s, 1H), 8.55 (s, 1H), 8.19-8.16 (m, 2H), 7.64 (d, J = 8.8 |
| | | Hz, 1H), 6.65 (s, 1H), 6.22 (s, 2H), 4.31-4.16 (m, 1H), 3.94 (s, 3H), 3.51- |
| | | 3.37 (m, 2H), 3.32 (s, 3H), 1.22 (d, J = 6.8 Hz, 3H). |
| 132 | 439.2 | ¹H NMR (400 MHz, DMSO-d₆, ppm) 9.55 (d, J = 8.0 Hz, 1H), 8.66 (s, 1H), |
| | | 8.59 (s, 1H), 8.52 (d, J = 1.2 Hz, 1H), 8.20 (d, J = 8.4 Hz, 1H), 8.15 (s, 1H), |
| | | 8.09 (dd, J = 8.4, 1.6 Hz, 1H), 4.28-4.18 (m, 1H), 3.94 (s, 3H), 3.47-3.39 (m, |
| | | 2H), 3.32 (s, 3H), 2.85 (s, 3H), 1.21 (d, J = 6.8 Hz, 3H). |
| 133 | 419.2 | ¹H NMR (400 MHz, DMSO-d₆, ppm) 9.55 (d, J = 8.0 Hz, 1H), 9.40 (s, 1H), |
| | | 8.82 (s, 1H), 8.74 (s, 1H), 8.62 (s, 1H), 8.59 (d, J = 5.6 Hz, 1H), 8.42 (dd, J = |
| | | 8.8, 1.6 Hz, 1H), 8.27 (d, J = 8.8 Hz, 1H), 8.21 (s, 1H), 8.03 (d, J = 5.6 Hz, |
| | | 1H), 4.32-4.17 (m, 1H), 3.95 (s, 3H), 3.52-3.37 (m, 2H), 3.33-3.32 (m, 3H), |
| | | 1.22 (d, J = 6.8 Hz, 3H). |
| 134 | 451.2 | ¹H NMR (400 MHz, DMSO-d₆, ppm) 9.54 (d, J = 8.0 Hz, 1H), 8.52 (s, 1H), |
| | | 8.47 (s, 1H), 7.92 (s, 1H), 7.53-7.47 (m, 2H), 7.34 (t, J = 7.6 Hz, 1H), 7.20 (t, |
| | | J = 7.6 Hz, 1H), 4.27-4.17 (m, 1H), 3.92 (s, 3H), 3.31-3.29 (m, 5H), 1.44 (s, |
| | | 6H), 1.20 (d, J = 6.7 Hz, 3H). |
| 135 | 386.2 | ¹H NMR (400 MHz, DMSO-d₆, ppm) 9.62 (d, J = 8.0 Hz, 1H), 8.63 (s, 1H), |
| | | 8.56 (s, 1H), 8.43 (s, 1H), 8.16 (s, 1H), 4.27-4.14 (m, 1H), 3.92 (s, 3H), |
| | | 3.46-3.37 (m, 2H), 3.31 (s, 3H), 2.23 (s, 3H), 2.06 (s, 3H), 1.20 (d, J = 6.8 |
| | | Hz, 3H). |
| 136 | 426.1 | ¹H NMR (400 MHz, DMSO-d₆, ppm) 9.54 (d, J = 8.4 Hz, 1H), 9.50 (s, 1H), |
| | | 8.65 (d, J = 4.0 Hz, 2H), 8.40 (s, 1H), 8.34 (s, 1H), 4.28-4.17 (m, 1H), 3.93 |
| | | (s, 2H), 3.47-3.37 (m, 1H), 3.31 (s, 2H), 1.20 (d, J = 6.8 Hz, 2H). |
| 137 | 397.2 | ¹H NMR (400 MHz, DMSO-d₆, ppm) 9.61 (d, J = 8.0 Hz, 1H), 8.63 (s, 1H), |
| | | 8.61 (d, J = 2.4 Hz, 1H), 8.58 (s, 1H), 8.18 (s, 1H), 6.42 (d, J = 2.6 Hz, 1H), |
| | | 4.28-4.15 (m, 1H), 3.91 (s, 3H), 3.46-3.37 (m, 2H), 3.31 (s, 3H), 2.09-2.00 |
| | | (m, 1H), 1.20 (d, J = 6.8 Hz, 3H), 1.02-0.94 (m, 2H), 0.85-0.75 (m, 2H). |
| 138 | 426.1 | ¹H NMR (400 MHz, DMSO-d₆, ppm) 9.53 (d, J = 8.0 Hz, 1H), 9.04 (s, 1H), |
| | | 8.62 (d, J = 5.2 Hz, 2H), 8.25 (s, 1H), 7.23 (d, J = 2.4 Hz, 1H), 4.24-4.17 (m, |
| | | 1H), 3.93 (s, 3H), 3.45-3.41 (m, 2H), 3.31 (s, 3H), 1.21 (d, J = 6.8 Hz, 3H). |
| 139 | 480.2 | ¹H NMR (400 MHz, DMSO-d₆, ppm) 9.58 (s, 2H), 9.49 (d, J = 8.0 Hz, 1H), |
| | | 8.76 (s, 1H), 8.65 (s, 1H), 8.48 (d, J = 8.8 Hz, 2H), 8.23 (s, 1H), 7.65 (d, J = |
| | | 8.8 Hz, 2H), 4.29-4.17 (d, J = 6.8 Hz, 1H), 3.94 (s, 3H), 3.48-3.38 (m, 2H), |
| | | 3.31 (s, 3H), 1.22 (d, J = 6.8 Hz, 3H). |
| 140 | 412.2 | ¹H NMR (400 MHz, DMSO-d₆, ppm) 9.56 (d, J = 8.3 Hz, 1H), 8.60 (s, 1H), |
| | | 8.52 (s, 1H), 8.13 (s, 1H), 7.65 (d, J = 1.6 Hz, 1H), 7.55 (dd, J = 8.0, 1.6 Hz, |
| | | 1H), 7.06 (d, J = 8.0 Hz, 1H), 6.13 (s, 2H), 4.25-4.15 (m, 1H), 3.92 (s, 3H), |
| | | 3.47-3.37 (m, 2H), 3.31 (s, 3H), 1.20 (d, J = 6.8 Hz, 3H). |
| 141 | 422.2 | ¹H NMR (400 MHz, DMSO-d₆, ppm) 9.57 (d, J = 8.0 Hz, 1H), 8.53 (d, J = |
| | | 4.8 Hz, 2H), 8.10 (s, 1H), 7.27-7.55 (m, 2H), 7.07 (s, 1H), 4.28-4.15 (m, 1H), |
| | | 3.93 (s, 3H), 3.47-3.37 (m, 2H), 3.35 (s, 2H), 3.31 (s, 3H), 2.94 (t, J = 8.0 |
| | | Hz, 2H), 2.82 (s, 3H), 1.20 (d, J = 6.8 Hz, 3H). |
| 142 | 468.2 | ¹H NMR (400 MHz, DMSO-d₆, ppm) 9.59 (d, J = 8.0 Hz, 1H), 8.84 (d, J = |
| | | 2.8 Hz, 1H), 8.75 (s, 1H), 8.60 (s, 1H), 8.22 (s, 1H), 7.99 (d, J = 8.8 Hz, 2H), |
| | | 7.54 (d, J = 8.8 Hz, 2H), 7.25 (d, J = 2.4 Hz, 1H), 4.29-4.17 (m, 1H), 3.93 (s, |
| | | 3H), 3.48-3.38 (m, 2H), 3.32 (s, 3H), 1.22 (d, J = 6.4 Hz, 3H). |
| 143 | 440.2 | ¹H NMR (400 MHz, DMSO-d₆, ppm) 9.49 (d, J = 8.0 Hz, 1H), 8.51 (s, 1H), |
| | | 8.48 (s, 1H), 7.99 (s, 1H), 6.94 (s, 1H), 4.29-4.17 (m, 1H), 3.93 (s, 3H), |
| | | 3.48-3.38 (m, 2H), 3.31 (s, 3H), 2.66 (s, 3H), 1.20 (d, J = 6.8 Hz, 3H). |
| 144 | 455.2 | ¹H NMR (400 MHz, DMSO-d₆, ppm) 9.53 (d, J = 8.4 Hz, 1H), 9.13 (d, J = |
| | | 2.8 Hz, 1H), 8.73-8.68 (m, 2H), 8.61 (s, 1H), 8.24-8.16 (m, 2H), 7.85 (dd, J = |
| | | 8.4, 4.4 Hz, 1H), 4.30-4.17 (m, 1H), 3.95 (s, 3H), 3.50-3.38 (m, 2H), 3.32 |
| | | (s, 3H), 1.22 (d, J = 6.8 Hz, 3H). |
| 145 | 429.3 | ¹H NMR (400 MHz, DMSO-d₆, ppm) 9.91 (d, J = 8.0 Hz, 1H), 8.39 (s, 1H), |
| | | 7.95 (s, 1H), 7.81 (s, 1H), 4.22-4.12 (m, 1H), 3.88 (s, 3H), 3.50 (t, J = 6.8 |
| | | Hz, 2H), 3.48-3.38 (m, 2H), 3.29 (s, 3H), 3.27 (s, 2H), 1.81 (t, J = 6.8 Hz, |
| | | 2H), 1.55-1.35 (m, 10H), 1.17 (d, J = 6.8 Hz, 3H). |
| 146 | 479.2 | ¹H NMR (400 MHz, DMSO-d₆, ppm) 10.08 (d, J = 7.2 Hz, 1H), 8.59 (d, J = |
| | | 6.4 Hz, 2H), 8.23-8.17 (m, 2H), 8.13 (s, 1H), 7.53 (d, J = 8.4 Hz, 2H), 5.05- |
| | | 4.95 (m, 1H), 3.94 (s, 3H), 3.08 (s, 3H), 2.89 (s, 3H), 1.33 (d, J = 6.8 Hz, |
| | | 3H). |
| 147 | 505.2 | ¹H NMR (400 MHz, DMSO-d₆, ppm) 10.10 (d, J = 7.2 Hz, 1H), 8.64 (s, 1H), |
| | | 8.59 (s, 1H), 8.17 (s, 1H), 8.14 (s, 2H), 7.86 (d, J = 8.4 Hz, 2H), 7.80 (d, J = |
| | | 8.4 Hz, 2H), 7.58 (s, 1H), 7.56 (s, 1H), 5.06-4.96 (m, 1H), 3.95 (s, 3H), 3.09 |
| | | (s, 3H), 2.89 (s, 3H), 1.34 (d, J = 6.8 Hz, 3H). |
| 148 | 464.1 | ¹H NMR (400 MHz, DMSO-d₆, ppm) 9.56 (d, J = 8.0 Hz, 1H), 8.64 (s, 1H), |
| | | 8.58 (s, 1H), 8.17 (s, 1H), 8.14 (d, J = 5.2 Hz, 2H), 7.86 (d, J = 8.4 Hz, 2H), |
| | | 7.80 (d, J = 8.4 Hz, 2H), 7.57 (d, J = 8.4 Hz, 2H), 4.96 (t, J = 5.2 Hz, 1H), |
| | | 4.12-4.02 (m, 1H), 3.94 (s, 3H), 3.52-3.43 (m, 2H), 3.32 (s, 3H), 1.20 (d, J = |
| | | 6.8 Hz, 3H). |
| 149 | 386.2 | ¹H NMR (400 MHz, DMSO-d₆, ppm) 9.54 (d, J = 8.0 Hz, 1H), 8.44 (s, 1H), |
| | | 8.37 (s, 1H), 7.96 (s, 1H), 6.68 (s, 1H), 4.29-4.17 (m, 1H), 3.90 (s, 3H), |
| | | 3.48-3.38 (m, 2H), 3.30 (s, 3H), 2.65 (d, J = 17.2 Hz, 1H), 2.43-2.30 (m, |
| | | 2H), 1.95-1.80 (m, 2H), 1.70 (s, 1H), 1.37-1.22 (m, 1H), 1.18 (d, J = 6.4 Hz, |
| | | 3H), 1.00 (d, J = 6.4 Hz, 3H). |
| 150 | 463.2 | ¹H NMR (400 MHz, DMSO-d₆, ppm) 10.06 (d, J = 7.2 Hz, 1H), 8.65 (s, 1H), |
| | | 8.59 (s, 1H), 8.29 (d, J = 8.4 Hz, 2H), 8.14 (s, 1H), 7.89 (d, J = 8.4 Hz, 2H), |
| | | 5.07-4.95 (m, 1H), 3.94 (s, 3H), 3.08 (s, 3H), 2.89 (s, 3H), 1.34 (d, J = 6.8 |
| | | Hz, 3H). |
| 151 | 407.2 | ¹H NMR (400 MHz, DMSO-d₆, ppm) 9.62 (d, J = 8.0 Hz, 1H), 8.54 (d, J = |
| | | 4.0 Hz, 2H), 8.13 (d, J = 8.4 Hz, 1H), 8.08 (d, J = 3.6 Hz, 1H), 8.04 (s, 1H), |
| | | 7.69 (d, J = 8.0 Hz, 1H), 7.40-7.35 (m, 1H), 7.24 (t, J = 7.6 Hz, 1H), 6.82 (d, |
| | | J = 3.6 Hz, 1H), 4.23 (s, 1H), 3.95 (s, 3H), 3.47-3.37 (m, 2H), 3.31 (s, 3H), |
| | | 1.22 (d, J = 6.8 Hz, 3H). |
| 152 | 388.2 | ¹H NMR (400 MHz, DMSO-d₆, ppm) 9.57 (dd, J = 8.0, 3.6 Hz, 1H), 8.42 (d, |
| | | J = 2.4 Hz, 1H), 8.07 (d, J = 4.8 Hz, 1H), 7.93 (d, J = 4.4 Hz, 1H), 4.24-4.13 |
| | | (m, 1H), 3.90 (d, J = 3.2 Hz, 3H), 3.42-3.35 (m, 2H), 3.29 (s, 3H), 2.95-2.63 |
| | | (m, 1H), 1.97-1.82 (m, 2H), 1.81-1.34 (m, 6H), 1.17 (d, J = 6.8 Hz, 3H), |
| | | 1.12-0.99 (m, 1H), 0.96-0.86 (m, 3H). |
| 153 | 419.2 | ¹H NMR (400 MHz, DMSO-d₆, ppm) 9.57 (d, J = 8.0 Hz, 1H), 9.49 (s, 1H), |
| | | 9.15 (s, 1H), 8.87 (s, 1H), 8.64 (s, 1H), 8.30 (s, 1H), 8.22 (t, J = 7.2 Hz, 2H), |
| | | 7.88 (t, J = 8.0 Hz, 1H), 7.82-7.73 (m, 1H), 4.28-4.18 (m, 1H), 3.97 (s, 3H), |
| | | 3.48-3.38 (m, 2H), 3.32 (s, 3H), 1.22 (d, J = 6.8 Hz, 3H). |
| 154 | 454.2 | ¹H NMR (400 MHz, DMSO-d₆, ppm) 12.45 (brs, 1H), 9.52 (d, J = 8.0 Hz, |
| | | 1H), 8.54 (d, J = 8.4 Hz, 1H), 8.47 (d, J = 2.0 Hz, 1H), 8.22 (s, 1H), 7.98 (s, |
| | | 1H), 7.67 (d, J = 12.4 Hz, 1H), 4.27-4.17 (m, 1H), 3.93 (s, 1H), 3.47-3.37 |
| | | (m, 2H), 3.31 (s, 3H), 1.20 (d, J = 6.8 Hz, 3H). |
| 155 | 468.2 | ¹H NMR (400 MHz, DMSO-d₆, ppm) 9.60 (d, J = 8.0 Hz, 1H), 9.27 (s, 1H), |
| | | 8.71 (s, 1H), 8.64 (s, 1H), 8.46 (s, 1H), 8.36 (s, 1H), 7.90 (d, J = 8.0 Hz, |
| | | 2H), 7.51 (d, J = 8.4 Hz, 2H), 4.30-4.18 (m, 1H), 3.95 (s, 3H), 3.48-3.38 (m, |
| | | 2H), 3.30 (s, 3H), 1.21 d, J = 6.4 Hz, 3H). |
| 156 | 479.2 | ¹H NMR (400 MHz, DMSO-d₆, ppm) 9.52 (d, J = 8.0 Hz, 1H), 9.33 (d, J = |
| | | 2.4 Hz, 1H), 8.68 (s, 1H), 8.60 (s, 1H), 8.55 (dd, J = 8.4, 2.4 Hz, 1H), 8.22 |
| | | (d, J = 8.8 Hz, 2H), 8.17 (s, 1H), 8.15 (d, J = 8.4 Hz, 1H), 7.59 (d, J = 8.4 |
| | | Hz, 2H), 4.23 (m, 1H), 3.94 (s, 3H), 3.48-3.38 (m, 2H), 3.32 (s, 1H), 1.21 (d, |
| | | J = 6.8 Hz, 3H). |
| 157 | 465.1 | ¹H NMR (400 MHz, DMSO-d₆, ppm) 9.52 (d, J = 8.0 Hz, 1H), 9.35 (d, J = |
| | | 2.4 Hz, 1H), 8.70 (s, 1H), 8.62 (s, 1H), 8.57 (dd, J = 8.4, 2.4 Hz, 2H), 8.23 |
| | | (d, J = 8.8 Hz, 2H), 8.19-8.13 (m, 2H), 7.60 (d, J = 8.4 Hz, 2H), 4.96 (t, J = |
| | | 5.2 Hz, 1H), 4.15-4.01 (m, 1H), 3.94 (s, 3H), 3.55-3.44 (m, 2H), 1.20 (d, J = |
| | | 6.8 Hz, 3H). |
| 158 | 409.2 | ¹H NMR (400 MHz, DMSO-d₆, ppm) 9.56 (d, J = 8.0 Hz, 1H), 8.92 (s, 1H), |
| | | 8.59 (s, 1H), 8.36 (d, J = 8.4 Hz, 1H), 8.29 (d, J = 8.4 Hz, 1H), 8.11 (s, 1H), |
| | | 7.86 (t, J = 7.6 Hz, 1H), 7.65 (t, J = 7.6 Hz, 1H), 4.33-4.20 (m, 1H), 3.97 (s, |
| | | 3H), 3.53-3.40 (m, 2H), 3.31 (s, 3H), 1.23 (d, J = 6.8 Hz, 3H). |
| 159 | 409.2 | ¹H NMR (400 MHz, DMSO-d₆, ppm) 9.49 (d, J = 8.4 Hz, 1H), 8.95 (s, 1H), |
| | | 8.54 (s, 1H), 8.12 (dd, J = 6.8, 3.2 Hz, 2H), 8.03 (s, 1H), 7.61 (dd, J = 6.8, |
| | | 3.2 Hz, 2H), 4.31-4.19 (m, 1H), 3.96 (s, 3H), 3.45 (t, J = 4.4 Hz, 2H), 3.31 |
| | | (s, 3H), 1.22 (d, J = 6.8 Hz, 3H). |
| 160 | 371.2 | ¹H NMR (400 MHz, MeOD, ppm) 8.73-8.63 (m, 1H), 8.37 (d, J = 5.6 Hz, |
| | | 1H), 8.28 (d, J = 3.6 Hz, 1H), 8.04 (d, J = 5.2 Hz, 1H), 6.35 (d, J = 2.4 Hz, |
| | | 1H), 4.37-4.20 (m, 1H), 3.91 (d, J = 2.0 Hz, 3H), 3.51 (d, J = 4.8 Hz, 2H), |
| | | 3.42 (s, 3H), 2.30 (d, J = 2.0 Hz, 3H), 1.31 (d, J = 6.8 Hz, 3H). |
| 161 | 454.1 | ¹H NMR (400 MHz, DMSO-d₆, ppm) 9.60 (d, J = 8.0 Hz, 1H), 9.28 (s, 1H), |
| | | 8.72 (s, 1H), 8.64 (s, 1H), 8.47 (s, 1H), 8.36 (s, 1H), 7.92 (s, 1H), 7.90 (s, |
| | | 1H), 7.52 (s, 1H), 7.50 (s, 1H), 4.97 (t, J = 5.2 Hz, 1H), 4.06 (s, 1H), 3.94 (s, |
| | | 3H), 3.48 (t, J = 4.9 Hz, 2H), 1.19 (d, J = 6.8 Hz, 3H). |
| 162 | 454.1 | ¹H NMR (400 MHz, DMSO-d₆, ppm) 9.58 (d, J = 8.0 Hz, 1H), 8.85 (d, J = |
| | | 2.8 Hz, 1H), 8.77 (s, 1H), 8.61 (s, 1H), 8.23 (s, 1H), 8.01 (d, J = 8.8 Hz, 2H), |
| | | 7.55 (d, J = 8.4 Hz, 2H), 7.25 (d, J = 2.4 Hz, 1H), 4.94 (t, J = 5.6 Hz, 1H), |
| | | 4.13-4.03 (m, 1H), 3.93 (s, 3H), 3.49 (t, J = 5.2 Hz, 2H), 1.20 (d, J = 6.8 Hz, |
| | | 3H) |
| 163 | 466.1 | ¹H NMR (400 MHz, DMSO-d6, ppm) 9.58 (s, 2H), 9.49 (d, J = 8.0 Hz, 1H), |
| | | 8.76 (s, 1H), 8.65 (s, 1H), 8.47 (d, J = 8.4 Hz, 2H), 8.23 (s, 1H), 7.65 (d, J = |
| | | 8.4 Hz, 2H), 4.96 (t, J = 5.2 Hz, 1H), 4.12-4.00 (m, 1H), 3.93 (s, 3H), 3.56- |
| | | 3.40 (m, 2H), 1.20 (d, J = 6.8 Hz, 3H). |
| 164 | 489.2 | ¹H NMR (400 MHz, DMSO-d₆, ppm) 10.35 (d, J = 6.0 Hz, 1H), 8.65 (s, 1H), |
| | | 8.58 (s, 1H), 8.28 (d, J = 8.2 Hz, 2H), 8.13 (s, 1H), 7.90 (d, J = 8.4 Hz, 2H), |
| | | 4.81 (dd, J = 10.8, 6.0 Hz, 1H), 3.95 (s, 3H), 3.75-3.65 (m, 1H), 3.25 (dd, J = |
| | | 14.8, 4.4 Hz, 1H), 2.97 (s, 3H), 2.02 (d, J = 13.2 Hz, 1H), 1.95-1.85 (m, |
| | | 1H), 1.82-1.69 (m, 2H), 1.47-1.30 (m, 2H). |
| 165 | 505.2 | ¹H NMR (400 MHz, DMSO-d₆, ppm) 10.36 (d, J = 5.6 Hz, 1H), 8.58 (d, J = |
| | | 10.0 Hz, 2H), 8.18 (d, J = 8.4 Hz, 2H), 8.12 (s, 1H), 7.53 (d, J = 8.4 Hz, 2H), |
| | | 4.81 (dd, J = 10.8, 6.0 Hz, 1H), 3.94 (s, 3H), 3.75-3.62 (m, 1H), 3.29-3.20 |
| | | (m, 1H), 2.96 (s, 3H), 2.10-1.65 (m, 4H), 1.50-1.32 (m, 2H). |
| 166 | 478.2 | ¹H NMR (400 MHz, DMSO-d₆, ppm) 9.80 (s, 1H), 9.46 (d, J = 1.6 Hz, 1H), |
| | | 8.77 (dd, J = 8.0, 1.6 Hz, 1H), 8.70 (s, 1H), 8.63 (s, 1H), 8.19 (s, 1H), 8.05 |
| | | (d, J = 8.4 Hz, 1H), 3.94 (s, 3H), 2.93 (s, 6H), 1.54 (s, 6H). |
| 167 | 477.2 | ¹H NMR (400 MHz, DMSO-d₆, ppm) 9.83 (s, 1H), 8.62 (s, 1H), 8.59 (s, 1H), |
| | | 8.31 (d, J = 8.4 Hz, 2H), 8.14 (s, 1H), 7.89 (d, J = 8.4 Hz, 2H), 3.94 (s, 3H), |
| | | 2.93 (d, J = 4.8 Hz, 6H), 1.54 (s, 6H). |
| 168 | 493.2 | ¹H NMR (400 MHz, DMSO-d₆, ppm) 9.84 (s, 1H), 8.57 (d, J = 5.6 Hz, 2H), |
| | | 8.21 (d, J = 8.8 Hz, 2H), 8.13 (s, 1H), 7.52 (d, J = 8.4 Hz, 2H), 3.93 (s, 3H), |
| | | 2.92 (s, 6H), 1.53 (s, 6H). |
| 169 | 449.2 | ¹H NMR (400 MHz, DMSO-d₆, ppm) 9.90 (d, J = 7.2 Hz, 1H), 8.65 (s, 1H), |
| | | 8.60 (s, 1H), 8.30 (d, J = 8.0 Hz, 2H), 8.14 (s, 1H), 8.12 (d, J = 4.4 Hz, 1H), |
| | | 7.89 (d, J = 8.4 Hz, 2H), 4.59-4.46 (m, J = 7.2 Hz, 1H), 3.94 (s, 3H), 2.63 (d, |
| | | J = 4.4 Hz, 3H), 1.35 (d, J = 6.8 Hz, 3H). |
| 170 | 465.1 | ¹H NMR (400 MHz, DMSO-d₆, ppm) 9.91 (d, J = 7.2 Hz, 1H), 8.59 (d, J = |
| | | 4.0 Hz, 2H), 8.21 (d, J = 8.0 Hz, 2H), 8.15-8.08 (m, 2H), 7.53 (d, J = 8.0 Hz, |
| | | 2H), 4.58-4.46 (m, 1H), 3.94 (s, 3H), 2.63 (d, J = 4.4 Hz, 3H), 1.34 (d, J = 6.8 |
| | | Hz, 3H). |
| 171 | 491.2 | ¹H NMR (400 MHz, DMSO-d₆, ppm) 9.93 (d, J = 7.6 Hz, 1H), 8.63 (s, 1H), |
| | | 8.60 (s, 1H), 8.14 (dd, J = 14.4, 7.6 Hz, 4H), 7.86 (d, J = 8.4 Hz, 2H), 7.80 |
| | | (d, J = 8.4 Hz, 2H), 7.57 (d, J = 8.4 Hz, 2H), 4.60-4.46 (m, 1H), 3.95 (s, 3H), |
| | | 2.63 (d, J = 4.4 Hz, 3H), 1.35 (d, J = 6.8 Hz, 3H). |
| 172 | 490.2 | ¹H NMR (400 MHz, DMSO-d₆, ppm) 10.33 (d, J = 6.0 Hz, 1H), 9.44 (d, J = |
| | | 2.0 Hz, 1H), 8.77-8.71 (m, 2H), 8.62 (s, 1H), 8.19 (s, 1H), 8.06 (d, J = 8.0 |
| | | Hz, 1H), 4.87-4.76 (m, J = 10.4, 6.0 Hz, 1H), 3.95 (s, 3H), 3.77-3.61 (m, |
| | | 1H), 3.29-3.22 (m, 1H), 2.97 (s, 3H), 2.06-1.96 (m, 1H), 1.94-1.84 (m, 1H), |
| | | 1.82-1.68 (m, 2H), 1.47-1.29 (m, 2H). |
| 173 | 463.2 | ¹H NMR (400 MHz, DMSO-d₆, ppm) 10.05 (s, 1H), 8.59 (d, J = 4.0 Hz, 2H), |
| | | 8.21 (d, J = 8.8 Hz, 2H), 8.19 (s, 1H), 7.87-7.81 (m, 1H), 7.52 (d, J = 8.8 Hz, |
| | | 2H), 3.94 (s, 3H), 2.60 (d, J = 4.5 Hz, 3H), 1.54 (s, 6H). |
| 174 | 479.2 | ¹H NMR (400 MHz, DMSO-d₆, ppm) 10.04 (s, 1H), 8.65 (s, 1H), 8.60 (s, |
| | | 1H), 8.31 (d, J = 8.0 Hz, 2H), 8.15 (s, 1H), 7.89 (d, J = 8.4 Hz, 2H), 7.87- |
| | | 7.82 (m, 1H), 3.95 (s, 3H), 2.60 (d, J = 4.4 Hz, 3H), 1.54 (s, 6H). |
| 175 | 422.1 | ¹H NMR (400 MHz, DMSO-d₆, ppm) 9.57 (d, J = 8.0 Hz, 1H), 8.79 (s, 1H), |
| | | 8.58 (s, 1H), 8.29 (s, 1H), 8.17 (s, 1H), 7.83 (s, 2H), 4.96 (s, 1H), 4.08 (s, |
| | | 3H), 4.05 (d, J = 4.4 Hz, 1H), 3.94 (s, 3H), 3.49 (d, J = 2.0 Hz, 2H), 2.51 (s, |
| | | 3H), 1.20 (d, J = 6.8 Hz, 3H). |
| 176 | 463.2 | ¹H NMR (400 MHz, DMSO-d₆, ppm) 10.11 (d, J = 6.8 Hz, 1H), 8.79 (s, 1H), |
| | | 8.60 (s, 1H), 8.29 (s, 1H), 8.18 (s, 1H), 7.83 (s, 2H), 5.06-4.95 (m, 1H), |
| | | 4.08 (s, 3H), 3.95 (s, 3H), 3.10 (s, 3H), 2.89 (s, 3H), 2.51 (s, 3H), 1.35 (d, |
| | | J = 6.8 Hz, 3H). |
| 177 | 506.1 | ¹H NMR (400 MHz, DMSO-d₆, ppm) 10.07 (d, J = 7.2 Hz, 1H), 9.33 (d, J = |
| | | 2.0 Hz, 1H), 8.69 (s, 1H), 8.62 (s, 1H), 8.55 (dd, J = 8.4, 2.4 Hz, 1H), 8.25- |
| | | 8.19 (m, 2H), 8.18 (d, J = 0.8 Hz, 1H), 8.15 (d, J = 8.4 Hz, 1H), 7.63-7.56 |
| | | (m, 2H), 5.01 (p, J = 6.8 Hz, 1H), 3.95 (s, 3H), 3.09 (s, 3H), 2.89 (s, 3H), |
| | | 1.34 (d, J = 6.8 Hz, 3H). |
| 178 | 480.2 | ¹H NMR (400 MHz, DMSO-d₆, ppm) 9.98 (d, J = 6.8 Hz, 1H), 8.68 (s, 1H), |
| | | 8.02 (d, J = 8.4 Hz, 2H), 7.66-7.62 (m, 2H), 7.58-7.53 (m, 3H), 7.42-7.37 (m, |
| | | 1H), 4.98 (p, J = 6.8 Hz, 1H), 3.07 (s, 3H), 3.00 (s, 3H), 2.92 (s, 3H), 2.87 |
| | | (s, 3H), 1.31 (d, J = 6.8 Hz, 3H). |
| 179 | 439.2 | ¹H NMR (400 MHz, DMSO-d₆, ppm) 9.44 (d, J = 7.6 Hz, 1H), 8.68 (s, 1H), |
| | | 8.02 (d, J = 8.4 Hz, 2H), 7.65-7.60 (m, 2H), 7.57-7.523(m, 3H), 7.39 (dd, J = |
| | | 11.6, 5.2 Hz, 1H), 4.93 (s, 1H), 4.13-3.98 (m, 1H), 3.45 (d, J = 3.6 Hz, 2H), |
| | | 3.00 (s, 3H), 2.92 (s, 3H), 1.17 (d, J = 6.8 Hz, 3H). |
| 180 | 466.1 | ¹H NMR (400 MHz, DMSO-d₆, ppm) 9.82 (d, J = 7.2 Hz, 1H), 8.67 (s, 1H), |
| | | 8.10 (d, J = 4.4 Hz, 1H), 8.02 (d, J = 8.4 Hz, 2H), 7.65-7.61 (m, 2H), 7.58- |
| | | 7.53 (m, 3H), 7.40 (t, J = 8.8 Hz, 1H), 4.52 (p, J = 6.8 Hz, 1H), 3.00 (s, 3H), |
| | | 2.92(s, 3H), 2.62 (d, J = 4.8 Hz, 3H), 1.32 (d, J = 6.8 Hz, 3H). |
| 181 | 493.1 | ¹H NMR (400 MHz, DMSO-d₆, ppm) 9.89 (d, J = 7.2 Hz, 1H), 9.59 (s, 2H), |
| | | 8.77 (s, 1H), 8.67 (s, 1H), 8.48 (d, J = 8.4 Hz, 2H), 8.25 (s, 1H), 8.13 (d, J = |
| | | 4.5 Hz, 1H), 7.65 (d, J = 8.8 Hz, 2H), 4.54 (p, J = 6.8 Hz, 1H), 3.95 (s, 3H), |
| | | 2.64 (d, J = 4.8 Hz, 3H), 1.36 (d, J = 6.8 Hz, 3H). |
| 182 | 507.1 | ¹H NMR (400 MHz, DMSO-d₆, ppm) 10.05 (d, J = 7.2 Hz, 1H), 9.58 (s, 2H), |
| | | 8.77 (s, 1H), 8.66 (s, 1H), 8.47 (d, J = 8.8 Hz, 2H), 8.24 (s, 1H), 7.65 (d, J = |
| | | 8.4 Hz, 2H), 5.01 (p, J = 6.8 Hz, 1H), 3.94 (s, 3H), 3.09 (s, 3H), 2.89 (s, 3H), |
| | | 1.34 (d, J = 6.8 Hz, 3H). |
| 183 | 492.1 | ¹H NMR (400 MHz, DMSO-d₆, ppm) 9.91 (d, J = 7.6 Hz, 1H), 9.34 (d, J = |
| | | 2.0 Hz, 1H), 8.69 (s, 1H), 8.63 (s, 1H), 8.56 (dd, J = 8.4, 2.4 Hz, 1H), 8.26- |
| | | 8.18 (m, 3H), 8.17-8.12 (m, 2H), 7.60 (d, J = 8.4 Hz, 2H), 4.54 (p, J = 6.8 |
| | | Hz, 1H), 3.95 (s, 3H), 2.64 (d, J = 4.8 Hz, 3H), 1.36 (d, J = 6.8 Hz, 3H). |
| 184 | 449.1 | ¹H NMR (400 MHz, DMSO-d₆, ppm) 9.53 (d, J = 7.6 Hz, 1H), 9.33 (d, J = |
| | | 2.0 Hz, 1H), 8.69 (s, 1H), 8.61 (s, 1H), 8.54 (dd, J = 8.4, 2.4 Hz, 1H), 8.25 |
| | | (dd, J = 8.8, 5.6 Hz, 2H), 8.18 (s, 1H), 8.13 (d, J = 8.4 Hz, 1H), 7.37 (t, J = |
| | | 8.8 Hz, 2H), 4.96 (t, J = 5.2 Hz, 1H), 4.14-3.99 (m, 1H), 3.94 (s, 3H), 3.56- |
| | | 3.41 (m, 2H), 1.20 (d, J = 6.8 Hz, 3H). |
| 185 | 431.1 | ¹H NMR (400 MHz, DMSO-d₆, ppm) 9.53 (d, J = 8.0 Hz, 1H), 9.34 (d, J = |
| | | 2.0 Hz, 1H), 8.69 (s, 1H), 8.61 (s, 1H), 8.54 (dd, J = 8.4, 2.4 Hz, 1H), 8.19 |
| | | (d, J = 7.2 Hz, 3H), 8.13 (d, J = 8.4 Hz, 1H), 7.56-7.47 (m, 3H), 4.96 (t, J = |
| | | 5.2 Hz, 1H), 4.11-4.02 (m, 1H), 3.94 (s, 3H), 3.53-3.44 (m, 2H), 1.20 (d, J = |
| | | 6.8 Hz, 3H). |
| 186 | 450.1 | ¹H NMR (400 MHz, DMSO-d₆, ppm) 9.55 (s, 2H), 9.49 (d, J = 8.0 Hz, 1H), |
| | | 8.75 (s, 1H), 8.64 (s, 1H), 8.51 (dd, J = 8.8, 5.6 Hz, 2H), 8.23 (s, 1H), 7.40 |
| | | (t, J = 8.8 Hz, 2H), 4.96 (t, J = 5.2 Hz, 1H), 4.12-4.01 (m, 1H), 3.93 (s, 3H), |
| | | 3.54-3.43 (m, 2H), 1.20 (d, J = 6.8 Hz, 3H). |
| 187 | 432.1 | ¹H NMR (400 MHz, DMSO-d₆, ppm) 9.57 (s, 2H), 9.50 (d, J = 7.6 Hz, 1H), |
| | | 8.76 (s, 1H), 8.65 (s, 1H), 8.48 (dd, J = 6.0, 2.0 Hz, 2H), 8.23 (s, 1H), 7.61- |
| | | 7.53 (m, 3H), 4.97 (t, J = 5.2 Hz, 1H), 4.12-3.98 (m, 1H), 3.94 (s, 3H), 3.49 |
| | | (t, J = 5.2 Hz, 2H), 1.20 (d, J = 6.8 Hz, 3H). |
| 188 | 496.1 | ¹H NMR (400 MHz, DMSO-d₆, ppm) 10.08 (d, J = 6.8 Hz, 1H), 8.86 (s, 1H), |
| | | 8.75 (s, 1H), 8.53 (s, 1H), 8.08 (d, J = 8.0 Hz, 2H), 8.02 (s, 1H), 7.63 (d, J = |
| | | 7.6 Hz, 2H), 5.06-4.96 (m, 1H), 3.96 (s, 3H), 3.09 (s, 3H), 2.89 (s, 3H), 1.35 |
| | | (d, J = 6.8 Hz, 3H). |
| 189 | 496.1 | ¹H NMR (400 MHz, DMSO-d₆, ppm) 10.09 (d, J = 7.6 Hz, 1H), 9.65 (s, 1H), |
| | | 8.78 (s, 1H), 8.67 (s, 1H), 8.34 (s, 1H), 8.10 (d, J = 8.4 Hz, 2H), 7.62 (d, J = |
| | | 8.4 Hz, 2H), 5.09-4.95 (m, 1H), 3.96 (s, 3H), 3.09 (s, 3H), 2.90 (s, 3H), 1.35 |
| | | (d, J = 6.8 Hz, 3H). |
| 190 | 482.1 | ¹H NMR (400 MHz, DMSO-d₆, ppm) 9.91 (d, J = 7.2 Hz, 1H), 8.86 (s, 1H), |
| | | 8.74 (s, 1H), 8.53 (s, 1H), 8.15 (d, J = 4.4 Hz, 1H), 8.08 (d, J = 8.4 Hz, 2H), |
| | | 8.02 (s, 1H), 7.63 (d, J = 8.4 Hz, 2H), 4.55 (t, J = 7.2 Hz, 1H), 3.95 (s, 3H), |
| | | 2.64 (d, J = 4.4 Hz, 3H), 1.35 (d, J = 6.8 Hz, 3H). |
| 191 | 449.3 | ¹H NMR (400 MHz, DMSO-d₆, ppm) 9.52 (d, J = 8.0 Hz, 1H), 9.35 (s, 1H), |
| | | 8.69 (s, 1H), 8.61 (s, 1H), 8.57 (d, J = 8.4 Hz, 1H), 8.19 (d, J = 8.4 Hz, 2H), |
| | | 8.06 (d, J = 7.6 Hz, 1H), 8.00 (d, J = 10.4 Hz, 1H), 7.59 (dd, J = 14.4, 8.0 |
| | | Hz, 1H), 7.37-7.29 (m, 1H), 4.96 (t, J = 5.2 Hz, 1H), 4.13-3.99 (m, 1H), 3.94 |
| | | (s, 3H), 3.55-3.43 (m, 2H), 1.20 (d, J = 6.8 Hz, 3H). |
| 192 | 449.2 | ¹H NMR (400 MHz, DMSO-d₆, ppm) 9.52 (d, J = 7.6 Hz, 1H), 9.39 (d, J = |
| | | 2.0 Hz, 1H), 8.70 (s, 1H), 8.62 (s, 1H), 8.58 (dd, J = 8.4, 2.4 Hz, 1H), 8.18 |
| | | (s, 1H), 8.07-8.01 (m, 1H), 7.99-7.93 (m, 1H), 7.58-7.51 (m, 1H), 7.43-7.34 |
| | | (m, 2H), 4.96 (t, J = 5.2 Hz, 1H), 4.12-4.02 (m, 1H), 3.94 (s, 3H), 3.52-3.44 |
| | | (m, 2H), 1.20 (d, J = 6.4 Hz, 3H). |
| 193 | 450.2 | ¹H NMR (400 MHz, DMSO-d₆, ppm) 9.60 (s, 2H), 9.49 (d, J = 8.0 Hz, 1H), |
| | | 8.77 (s, 1H), 8.65 (s, 1H), 8.33 (d, J = 7.6 Hz, 1H), 8.24 (s, 1H), 8.16 (d, J = |
| | | 10.4 Hz, 1H), 7.67-7.61 (m, 1H), 7.47-7.42 (m, 1H), 4.96 (t, J = 5.6 Hz, 1H), |
| | | 4.10-4.04 (m, 1H), 3.94 (s, 3H), 3.50-3.47 (m, 2H), 1.20 (d, J = 6.8 Hz, 3H). |
| 194 | 450.2 | ¹H NMR (400 MHz, DMSO-d₆, ppm) 9.62 (s, 2H), 9.49 (d, J = 8.0 Hz, 1H), |
| | | 8.78 (s, 1H), 8.66 (s, 1H), 8.24 (s, 1H), 8.17-8.10 (m, 1H), 7.66-7.58 (m, |
| | | 1H), 7.44-7.35 (m, 2H), 4.96 (t, J = 5.2 Hz, 1H), 4.15-4.00 (m, 1H), 3.94 (s, |
| | | 3H), 3.55-3.42 (m, 2H), 1.20 (d, J = 6.8 Hz, 3H). |
| 195 | 450.2 | ¹H NMR (400 MHz, DMSO-d₆, ppm) 9.52 (d, J = 8.0 Hz, 1H), 9.37 (d, J = |
| | | 2.0 Hz, 1H), 9.04 (d, J = 2.4 Hz, 1H), 8.77-8.68 (m, 2H), 8.63-8.58 (m, 2H), |
| | | 8.23 (d, J = 8.0 Hz, 1H), 8.19 (s, 1H), 7.37 (dd, J = 8.8, 2.8 Hz, 1H), 4.96 (t, |
| | | J = 5.2 Hz, 1H), 4.13-4.02 (m, 1H), 3.94 (s, 3H), 3.53-3.43 (m, 2H), 1.20 (d, |
| | | J = 6.8 Hz, 3H). |
| 196 | 482.3 | ¹H NMR (400 MHz, DMSO-d₆, ppm) 9.52 (s, 1H), 9.41 (s, 1H), 8.96 (dd, J = |
| | | 4.4, 1.6 Hz, 1H), 8.85 (s, 1H), 8.73 (s, 1H), 8.65-8.56 (m, 3H), 8.54 (d, J = |
| | | 8.0 Hz, 1H), 8.33 (d, J = 8.4 Hz, 1H), 8.20 (s, 1H), 8.16 (d, J = 8.8 Hz, 1H), |
| | | 7.61 (dd, J = 8.4, 4.4 Hz, 1H), 4.97 (s, 1H), 4.15-4.00 (m, 1H), 3.95 (s, 3H), |
| | | 3.53-3.42 (m, 2H), 1.21 (d, J = 6.8 Hz, 3H). |
| 201 | 382.2 | ¹H NMR (400 MHz, DMSO-d₆, ppm) 9.56 (d, J = 8.0 Hz, 1H), 8.56 (d, J = |
| | | 3.2 Hz, 2H), 8.10 (s, 1H), 7.96 (d, J = 8.4 Hz, 2H), 7.39 (d, J = 8.4 Hz, 2H), |
| | | 4.95 (t, J = 5.2 Hz, 1H), 4.12-3.98 (m, 1H), 3.93 (s, 3H), 3.55-3.42 (m, 2H), |
| | | 2.74-2.62 (m, 2H), 1.28-1.13 (m, 6H). |
| 202 | 396.3 | ¹H NMR (400 MHz, DMSO-d₆, ppm) 9.56 (d, J = 8.0 Hz, 1H), 8.56 (d, J = |
| | | 2.8 Hz, 2H), 8.10 (s, 1H), 7.96 (d, J = 8.4 Hz, 2H), 7.42 (d, J = 8.4 Hz, 2H), |
| | | 4.95 (t, J = 5.2 Hz, 1H), 4.11-3.98 (m, 1H), 3.93 (s, 3H), 3.53-3.41 (m, 2H), |
| | | 3.08-2.86 (m, 1H), 1.25 (d, J = 6.8 Hz, 6H), 1.19 (d, J = 6.4 Hz, 3H). |
| 203 | 410.3 | ¹H NMR (400 MHz, DMSO-d₆, ppm) 9.57 (d, J = 8.0 Hz, 1H), 8.56 (d, J = |
| | | 2.8 Hz, 2H), 8.09 (s, 1H), 7.97 (d, J = 8.4 Hz, 2H), 7.57 (d, J = 8.4 Hz, 2H), |
| | | 4.95 (t, J = 5.2 Hz, 1H), 4.12-3.98 (m, 1H), 3.93 (s, 3H), 3.53-3.42 (m, 2H), |
| | | 1.33 (s, 9H), 1.19 (d, J = 6.4 Hz, 3H). |
| 204 | 396.3 | ¹H NMR (400 MHz, DMSO-d₆, ppm) 9.57 (d, J = 8.0 Hz, 1H), 8.55 (d, J = |
| | | 4.0 Hz, 2H), 8.10 (s, 1H), 7.96 (d, J = 8.4 Hz, 2H), 7.39 (d, J = 8.4 Hz, 2H), |
| | | 4.29-4.15 (m, 1H), 3.93 (s, 3H), 3.49-3.37 (m, 2H), 3.31 (s, 3H), 2.79-2.57 |
| | | (m, 2H), 1.29-1.09 (m, 6H). |
| 205 | 410.3 | ¹H NMR (400 MHz, DMSO-d₆, ppm) 9.57 (d, J = 8.0, 1H), 8.55 (d, J = 3.6, |
| | | 2H), 8.09 (s, 1H), 7.96 (d, J = 8.4, 2H), 7.56 (d, J = 8.4, 2H), 4.36-4.15 (m, |
| | | 1H), 3.93 (s, 3H), 3.48-3.39 (m, 2H), 3.31 (s, 3H), 1.33 (s, 9H), 1.21 (d, |
| | | J = 6.8, 3H). |
| 206 | 446.3 | ¹H NMR (400 MHz, DMSO-d₆, ppm) 9.57 (d, J = 8.0 Hz, 1H), 8.55 (d, J = |
| | | 3.6 Hz, 2H), 8.09 (s, 1H), 7.96 (d, J = 8.4 Hz, 2H), 7.42 (d, J = 8.4 Hz, 2H), |
| | | 4.29-4.15 (m, 1H), 3.93 (s, 3H), 3.46-3.38 (m, 2H), 3.31 (s, 4H), 3.08-2.86 |
| | | (m, 1H), 1.25 (d, J = 6.8 Hz, 8H), 1.20 (d, J = 6.8 Hz, 3H). |
| 207 | 455.3 | ¹H NMR (400 MHz, DMSO-d₆, ppm) 9.80 (d, J = 8.0, 1H), 8.41 (s, 1H), 8.13 |
| | | (s, 1H), 7.96 (s, 1H), 7.42-7.23 (m, 2H), 7.21-7.00 (m, 2H), 4.92 (t, J = |
| | | 5.2, 1H), 4.17 (d, J = 12.8, 2H), 4.08-3.94 (m, 1H), 3.88 (s, 3H), 3.50-3.38 |
| | | (m, 2H), 3.06-2.89 (m, 2H), 2.89-2.75 (m, 1H), 1.86 (d, J = 11.2, 2H), 1.77- |
| | | 1.63 (m, 2H), 1.16 (d, J = 6.8, 3H). |
| 208 | 456.2 | ¹H NMR (400 MHz, DMSO-d₆, ppm) 9.76 (d, J = 8.0, 1H), 8.42 (s, 1H), 8.16 |
| | | (s, 1H), 7.97 (s, 1H), 7.26-6.91 (m, 4H), 4.91 (t, J = 5.2, 1H), 4.02 (s, 1H), |
| | | 3.89 (s, 3H), 3.56 (s, 4H), 3.50-3.38 (m, 2H), 3.23 (d, J = 5.2, 4H), 1.17 (d, J = |
| | | 6.8, 3H). |
| 209 | 456.1 | ¹H NMR (400 MHz, DMSO-d₆, ppm) 9.77 (d, J = 7.9 Hz, 1H), 8.43 (s, 1H), |
| | | 8.15 (s, 1H), 7.99 (s, 1H), 7.20-7.07 (m, 3H), 7.04-6.98 (m, 1H), 4.92 (t, |
| | | J = 5.3 Hz, 1H), 4.06-3.99 (m, 1H), 3.89 (s, 3H), 3.60-3.56 (m, 4H), 3.45 |
| | | (dd, J = 7.8, 5.0 Hz, 2H), 3.18-3.13 (m, 4H), 1.17 (d, J = 6.7 Hz, 3H). |
| 210 | 456.0 | ¹H NMR (400 MHz, DMSO-d6 , ppm) 9.77 (d, J = 7.9 Hz, 1H), 8.43 (s, 1H), |
| | | 8.16 (s, 1H), 7.97 (s, 1H), 7.28-7.22 (m, 1H), 6.82 (dd, J = 9.8, 5.6 Hz, |
| | | 2H), 6.58 (dd, J = 8.2, 2.2 Hz, 1H), 4.93 (t, J = 5.3 Hz, 1H), 4.06-3.99 (m, |
| | | 1H), 3.89 (s, 3H), 3.81 (s, 1H), 3.57-3.53 (m, 4H), 3.45 (dd, J = 8.1, 5.0 |
| | | Hz, 2H), 3.35 (s, 2H), 3.25 (s, 1H), 1.17 (d, J = 6.7 Hz, 3H). |

### Example 5—AHR Antagonist Reporter Assay in Human Cell Line

The AhR reporter assay was performed following the protocol of the Human Aryl Hydrocarbon Receptor (AhR) Reporter Assay System (INDIGO Bioscience, #1B06001-32). AhR Reporter Cell was thawed and pre-incubated in Cell Recovery Medium (CRM) for 6 hours. The media was then removed and the cells were incubated with Compound Screening Medium (CSM) supplemented with 200 μM kynurenic acid for 24 hours in the absence (negative control) or presence of increasing concentrations of test compounds (typical dilution: 128 μM, 640 μM, 3.2 nM, 16 nM, 80 nM, 400 nM, 2 μM, 10 μM in duplicates). For positive inhibition control, cells activated with 200 μM kynurenic acid were incubated in the presence of literature AHR antagonists; alternatively un-activated cells were used as positive inhibition control. Following 24 hours incubation, the media was removed and Luciferase Detection Reagent (LDR) added. The firefly luciferase activity was quantified using a plate reader. Results were normalized by positive and negative controls.

### Example 6—AHR-Regulated CYP1A1 Expression in Human Cell Line

To evaluate the AHR inhibitory activity of the test compounds, quantitative PCR analysis was used to determine expression level of the AHR-regulated gene CYP1A1 in a human monocytic U937 cell line upon stimulation of 200 μM kynurenic acid (KA) in the absence or presence of AHR inhibitor. U937 cells were seeded at a concentration of 5×10⁵ cells/well in 200 μL of growth medium in 96-well cell culture microplates. CYP1A1 expression was induced with 200 μM KA (positive control) in the absence or presence of the test compounds for 16 hours. Human U937 cells were typically incubated with eight different concentrations of test compounds (typical dilution: 128 μM, 640 μM, 3.2 nM, 16 nM, 80 nM, 400 nM, 2 μM, 10 μM) and analyzed in duplicate on the same cell culture microplate. After stimulation, the media was removed, the cell RNA was isolated using RNeasy Mini Kit (Qiagen, 74104) and reverse-transcribed to cDNA using PrimeScript RT reagent Kit (Perfect Real Time) (TaKaRa, RR037A). Unstimulated cells were used as the negative control. Taqman probes for human CYP1A1 (Hs01054797_g1) and human GAPDH (Hs02786624_g1) were used to analyze fold expression of CYP1A1 of GAPDH.

Results are shown in Table 4, below.

TABLE 4

| | AhR | Cyp |
|---|---|---|
| | Reporter | ANATAG |
| | Assay KA | mode |
| | Antagonist | assay: |
| | Mode: | IC50 |
| Cmp. # | IC50 (nM) | (nM) |
| 2 | B | |
| 3 | A | |
| 4 | A | |
| 5 | A | |
| 6 | A | |
| 7 | C | |
| 8 | A | |
| 9 | A | |
| 10 | A | |
| 11 | A | |
| 12 | B | B |
| 13 | C | B |
| 14 | A | |
| 15 | A | |
| 16 | A | |
| 17 | A | |
| 18 | A | |
| 19 | A | |
| 20 | A | |
| 21 | A | |
| 22 | A | |
| 23 | A | |
| 24 | A | |
| 25 | ND | |
| 26 | C | |
| 28 | C | |
| 28 - | C | C |
| *stereo- | | |
| pure | | |
| 30 | A | |
| 31 | A | |
| 32 | C | C |
| 33 | A | |
| 34 | A | |
| 35 | A | |
| 36 | A | |
| 37 | A | |
| 38 | C | |
| 39 | C | C |
| 40 | C | |
| 41 | A | |
| 42 | A | |
| 43 | A | |
| 44 | B | |
| 45 | A | |
| 46 | A | |
| 47 | A | |
| 48 | A | |
| 49 | A | |
| 50 | A | |
| 51 | A | |
| 52 | A | |
| 53 | A | |
| 54 | A | |
| 55 | A | |
| 56 | B | B |
| 57 | C | |
| 58 | A | B |
| 59 | C | |
| 59 - | C | C |
| *stereo- | | |
| pure | | |
| 61 | A | |
| 62 | B | C |
| 64 | A | |
| 65 | A | |
| 66 | A | |
| 67 | A | |
| 67 | C | |
| 68 | B | |
| 69 | C | |
| 70 | C | C |
| 71 | C | |
| 72 | B | |
| 73 | C | C |
| 74 | B | |
| 75 | A | |
| 76 | C | C |
| 77 | A | |
| 78 | B | |
| 79 | A | |
| 80 | A | |
| 81 | A | C |
| 82 | C | |
| 83 | A | |
| 85 | A | |
| 87 | A | |
| 88 | C | |
| 89 | B | |
| 90 | C | C |
| 91 | A | |
| 94 | C | C |
| 95 | C | C |
| 96 | C | |
| 97 | C | C |
| 98 | C | C |
| 99 | C | C |
| 100 | C | C |
| 101 | C | C |
| 102 | A | |
| 103 | A | |
| 104 | C | |
| 105 | A | |
| 106 | C | C |
| 107 | A | |
| 108 | A | |
| 109 | A | |
| 110 | A | |
| 111 | A | |
| 112 | C | B |
| 113 | C | C |
| 114 | A | |
| 115 | A | |
| 116 | A | |
| 117 | A | |
| 118 | A | |
| 119 | A | |
| 120 | C | C |
| 121 | A | |
| 122 | A | |
| 123 | C | C |
| 124 | A | |
| 125 | C | |
| 126 | A | |
| 127 | A | |
| 128 | A | |
| 129 | A | |
| 130 | B | |
| 131 | A | |
| 132 | A | |
| 133 | A | |
| 134 | A | |
| 135 | A | |
| 136 | A | |
| 137 | A | |
| 138 | A | |
| 139 | C | C |
| 140 | A | |
| 141 | A | |
| 142 | C | |
| 143 | A | |
| 144 | A | |
| 145 | A | |
| 146 | C | C |
| 147 | C | C |
| 148 | C | C |
| 149 | C | |
| 150 | C | C |
| 151 | A | |
| 152 | A | |
| 153 | C | C |
| 154 | A | |
| 155 | C | B |
| 156 | C | C |
| 157 | C | C |
| 158 | A | |
| 159 | B | |
| 160 | A | |
| 161 | C | C |
| 162 | C | |
| 163 | C | C |
| 164 | C | C |
| 165 | C | C |
| 166 | A | |
| 167 | C | C |
| 168 | C | B |
| 169 | C | C |
| 170 | C | C |
| 171 | C | C |
| 172 | C | C |
| 173 | C | C |
| 174 | B | |
| 175 | C | B |
| 176 | C | B |
| 177 | C | C |
| 178 | A | |
| 179 | A | |
| 180 | C | |
| 181 | C | B |
| 182 | C | |
| 183 | C | C |
| 184 | C | C |
| 185 | C | C |
| 186 | C | C |
| 187 | C | |
| 188 | A | |
| 189 | A | |
| 190 | A | |
| 190 | A | |
| 191 | C | C |
| 192 | C | C |
| 193 | C | C |
| 194 | C | C |
| 195 | C | C |
| 196 | A | |
| 197 | B | B |
| 198 | C | C |
| 199 | C | C |
| 200 | C | C |
| 201 | C | C |
| 202 | C | C |
| 203 | C | C |
| 204 | B | C |
| 205 | A | A |
| 206 | C | C |
| 207 | A | B |
| 208 | C | C |
| 209 | C | |
| 210 | C | |

A >400 nM;
B 100 to 400 nM;
C <100 nM

### Example 7—Evaluation of Anti-Tumor Activity

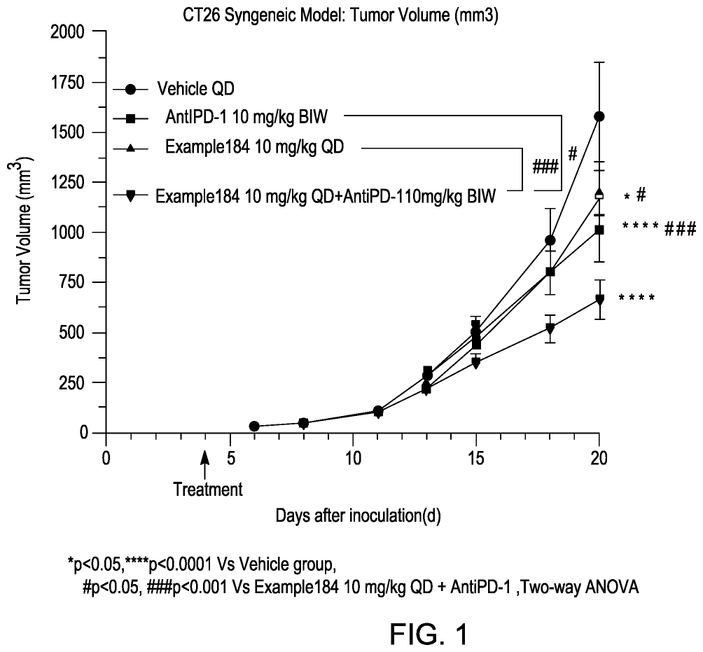

Female BALB/C mouse was inoculated subcutaneously on the right flank with CT26 tumor cells (3×10⁵) in 0.1 ml of RPMI 1640 Medium without serum for tumor development. Mice were randomized into 4 groups according to body weight on Day 4 after inoculation. The tumor-bearing mice were treated with Vehicle, anti PD-1 antibody (RMP1-14, BioXCell Catalog No. BP0146), a AHR antagonist compound as disclosed herein and a combination therapy of a AHR antagonist compound as disclosed herein and the anti PD-1 (or established treatment). The AHR antagonist compound was dosed orally at for example 10 mg/kg alone or in combination with established treatment for 16 days. When the established treatment was PD-1 antibody, the antibody was administered via intraperitoneal injection at 10 mg/kg, twice every week. The result of the tumor growth inhibition using Example 184 as AHR antagonist compound is shown in FIG. 1. All treatment groups showed significantly reduced tumor volume compared with Vehicle group, the p values of anti PD-1 antibody group, example 184 group and combination group were <0.0001, 0.0083, and <0.0001, respectively. Compared with anti PD-1 antibody group and example 184 group, the combination group resulted in a significantly reduction in tumor volume, with p values of 0.0235 and <0.0001, respectively.

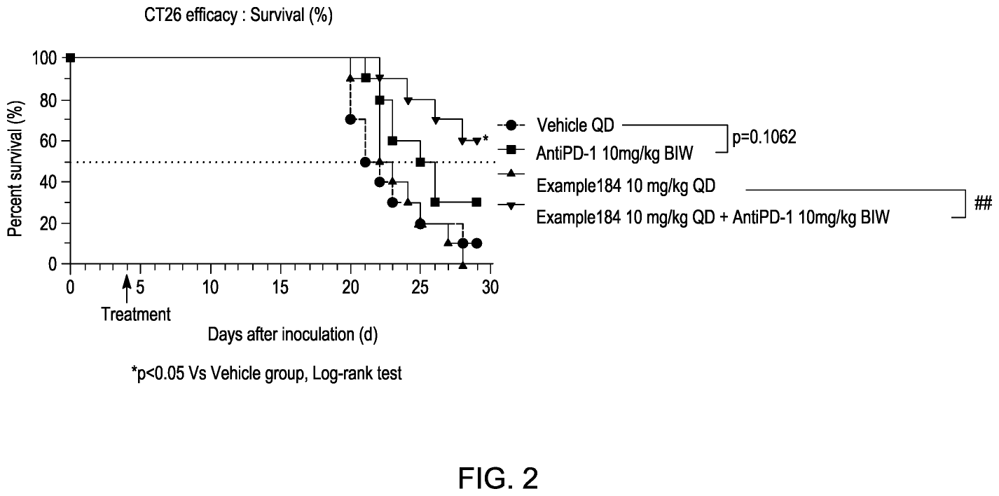

The synergistic tumor growth inhibition of example 184 and anti-PD-1 antibody also translated into significant survival benefit as shown in the survival curve of FIG. 2, with p values of 0.0049 against vehicle and 0.0011 against example 184 standalone group.

It should be appreciated that all combinations of the foregoing concepts and implementations and additional concepts and implementations discussed in greater detail below are contemplated as being part of the inventive subject matter disclosed herein, and may be employed in any suitable combination to achieve the benefits as described here. In particular, all combinations of claimed subject matter appearing at the end of this disclosure are contemplated as being part of the inventive subject matter disclosed herein. The foregoing description is given for clearness of understanding only, and no unnecessary limitations should be understood therefrom, as modifications within the scope of the disclosure may be apparent to those having ordinary skill in the art.

The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The disclosure illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular implementations, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

We claim:

1. A compound, or pharmaceutically acceptable salt thereof, having a structure of Formula (I): 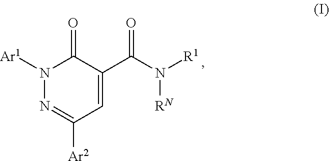
wherein
each R^{N} is independently H or C₁₋₆alkyl;
R¹ is H, C₁₋₆alkyl, C₁₋₆alkylene-NR^{N}R^{N}, C₁₋₆alkylene-O—C₁₋₆alkyl, C₁₋₆alkylene-C(O)R², C₁₋₆alkylene-NR^{N}—C(O)—C₁₋₃alkyl, C₃₋₈cycloalkyl, or 4-12 membered heterocyclyl, wherein 1-3 ring atoms are selected from O, N, and S, and the cycloalkyl or heterocyclyl is optionally substituted with 1 or 2 C₁₋₆alkyl, C(O)—C₁₋₆alkyl, and ═O;
R² is OH, O—C₁₋₆alkyl, or NR^{N}R^{N};
Ar¹ is C₆₋₁₀aryl or 5-10 membered heteroaryl, wherein 1-3 ring atoms are selected from O, N, and S, and Ar¹ is optionally substituted with 1 or 2 R³;
each R³ is independently halo, C₁₋₆alkyl, C₁₋₆haloalkyl, or C₃₋₆cycloalkyl;
Ar² is C₅₋₈cycloakly, C₅₋₈cycloalkenyl, C₆₋₁₀aryl, or 5-12 membered heterocyclyl or heteroaryl, wherein 1-3 ring atoms are selected from O, N, and S, and Ar² is optionally substituted with 1, 2, or 3 R⁴;
each R⁴ is independently halo, OH, ═O, CN, C₁₋₆alkyl, C₁₋₆haloalkyl, C₁₋₆hydroxyalkyl, C₁₋₆alkoxy, C₁₋₆haloalkoxy, NR^{N}R^{N}, CONR^{N}R^{N}, COOH, COO—C₁₋₆alkyl, C(O)—C₁₋₆alkyl, SO₂—C₁₋₆alkyl, C₃₋₆cycloalkyl, 4-12 membered heterocyclyl or 5-12 membered heteroaryl, wherein 1-3 ring atoms are selected from O, N, and S, or phenyl, and the heterocyclyl, heteroaryl, or phenyl is substituted with 0, 1, or 2 substituents independently selected from halo and C₁₋₆alkyl,
or R¹ can be C₂₋₆hydroxyalkyl when Ar² is (1) C₅₋₈cycloakly, C₅₋₈cycloalkenyl, C₁₀aryl, 4-12-membered heterocyclyl, or 5- or 7-12 membered heteroaryl and Ar² is optionally substituted with 1, 2, or 3 R⁴; or (2) phenyl or 6-membered heteroaryl and Ar² is substituted with at least one R⁴ selected from CONR^{N}R^{N}, COOH, COO—C₁₋₆alkyl, C(O)—C₁₋₆alkyl, SO₂—C₁₋₆alkyl, C₃₋₆cycloalkyl, 4-12 membered heterocyclyl, 5-12 membered heteroaryl, and phenyl, and the phenyl, heterocyclyl or heteroaryl is substituted with 0, 1, or 2 substituents independently selected from halo and C₁₋₆alkyl.

2. The compound or salt of claim 1, wherein each R^{N} is independently H or methyl.

3. The compound or salt of claim 1 or 2, wherein R¹ is H or C₁₋₆alkyl.

4. The compound or salt of claim 3, wherein R¹ is methyl.

5. The compound or salt of claim 1 or 2, wherein R¹ is C₁₋₆alkylene-NR^{N}R^{N} or C₁₋₆alkylene-O—C₁₋₆alkyl.

6. The compound or salt of claim 5, wherein R¹ is

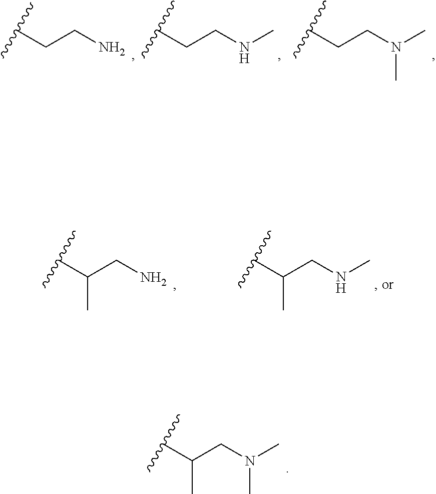

7. The compound or salt of claim 5, wherein R¹ is

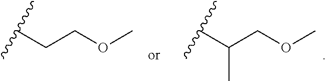

8. The compound or salt of claim 7, wherein R¹ is

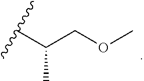 [SMILES: *[C@@H](C)COC]

9. The compound or salt of claim 1 or 2, wherein R¹ is C₁₋₆alkylene-C(O)R² or C₁₋₆alkylene-NR^{N}—C(O)CH₃.

10. The compound or salt of claim 9, wherein R² is OH.

11. The compound or salt of claim 9, wherein R² is NH₂, NHCH₃, or N(CH₃)₂.

12. The compound or salt of claim 9, wherein R¹ is or

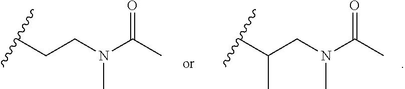

13. The compound or salt of claim 9, wherein R¹ is

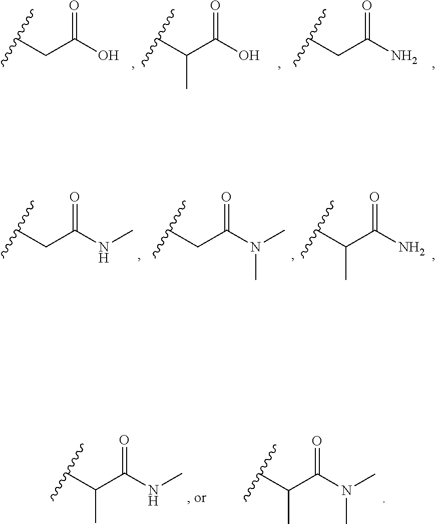

14. The compound or salt of claim 1 or 2, wherein R¹ is heterocyclyl comprising 4-12 total ring atoms, wherein 1-3 of the ring atoms are selected from O, N, and S.

15. The compound or salt of claim 12, wherein the heterocyclyl is unsubstituted.

16. The compound or salt of claim 12, wherein the heterocyclyl is substituted with 1 or 2 C₁₋₆alkyl, C(O)—C₁₋₆alkyl, or ═O.

17. The compound or salt of claim 14, wherein R¹ is

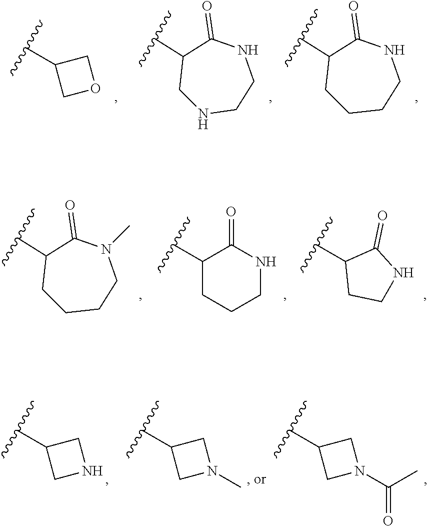

18. The compound or salt of claim 17, wherein R¹ is

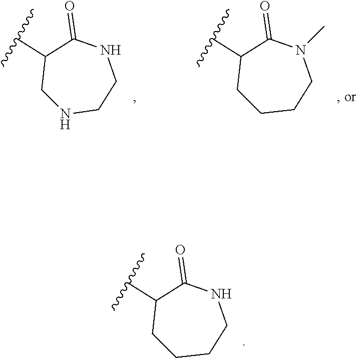

19. The compound or salt of claim 1 or 2, wherein R¹ is C₂₋₆alkylene-C(O)—NR^{N}R^{N}.

20. The compound or salt of claim 19, wherein R¹ is

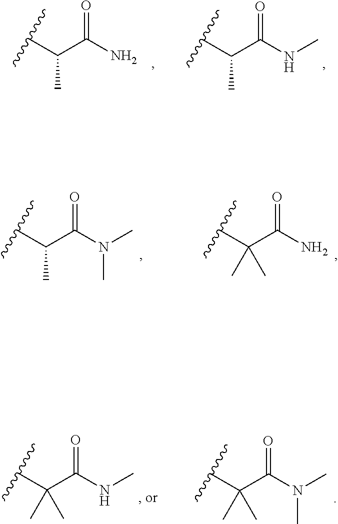

21. The compound or salt of claim 1 or 2, wherein R¹ is C₂₋₆hydroxyalkyl.

22. The compound or salt of claim 21, wherein R¹ is

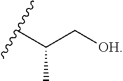 [SMILES: *[C@@H](C)CO]

23. The compound or salt of any one of claims 1 to 22, wherein Ar¹ is C₆₋₁₀aryl.

24. The compound or salt of claim 23, wherein Ar¹ is phenyl.

25. The compound or salt of any one of claims 1 to 22, wherein Ar¹ is 5-10 membered heteroaryl.

26. The compound or salt of any one of claims 23 to 25, wherein Ar¹ is unsubstituted.

27. The compound or salt of claim 26, wherein Ar¹ is

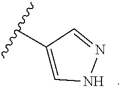 [SMILES: *c1cn[nH]c1]

28. The compound or salt of any one of claims 23 to 25, wherein Ar¹ is substituted with 1 or 2 R³.

29. The compound or salt of claim 28, wherein R³ is fluoro, chloro, methyl, ethyl, isopropyl, or trifluoromethyl.

30. The compound or salt of claim 28 or 29, wherein Ar¹ is

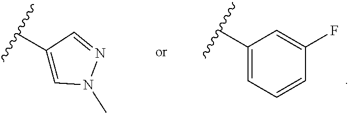

31. The compound or salt of claim 30, wherein Ar¹ is

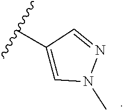 [SMILES: *c1cnn(C)c1]

32. The compound or salt of any one of claims 1 to 31, wherein Ar² is C₆₋₁₀aryl.

33. The compound or salt of claim 32, wherein Ar² is phenyl.

34. The compound or salt of any one of claims 1 to 31, wherein Ar² is 5-12 membered heteroaryl.

35. The compound or salt of any one of claims 1 to 31, wherein Ar² is C₅₋₈cycloalkyl, C₅₋₈cycloalkenyl, or 5-12 membered heterocyclyl.

36. The compound or salt of claim 35, wherein Ar² is 5-12 membered heterocyclyl.

37. The compound or salt of claim 32, 34, 35, or 36, wherein Ar² is unsubstituted.

38. The compound or salt of any one of claims 32 to 36, wherein Ar² is substituted with 1, 2, or 3 R⁴.

39. The compound or salt of claim 38, wherein Ar² is substituted with 1 or 2 R⁴.

40. The compound or salt of claim 38 or 39, wherein at least one R⁴ is halo.

41. The compound or salt of claim 40, wherein at least one R⁴ is chloro.

42. The compound or salt of claim 41, wherein Ar² is Cl

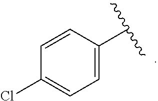 [SMILES: *c1ccc(Cl)cc1]

43. The compound or salt of any one of claims 38 to 41, wherein at least one R⁴ is fluoro, chloro, OH, CN, CH₃, CH₂CH₃, CH(CH₃)₂, C(CH₃)₃, OCH₃, CF₃, OCF₃, CHF₂, CH₂F, OCHF₂, NH₂, N(CH₃)₂, CONH₂, COOH, SO₂CH₃, cyclopropyl, or morpholino.

44. The compound or salt of any one of claims 38 to 41 and 43, wherein at least one R⁴ selected from CONR^{N}R^{N}, COOH, COO—C₁₋₆alkyl, C(O)—C₁₋₆alkyl, SO₂—C₁₋₆alkyl, C₃₋₆cycloalkyl, 4-12 membered heterocyclyl, 5-12 membered heteroaryl, and phenyl, and the phenyl, heterocyclyl or heteroaryl is substituted with 0, 1, or 2 substituents independently selected from halo and C₁₋₆alkyl.

45. The compound or salt of claim 44, wherein Ar² is phenyl, pyridyl, pyrimidinyl, pyrazolyl, or triazolyl, and at least one R⁴ is phenyl or halo substituted-phenyl.

46. The compound or salt of any one of claims 1 to 31 wherein Ar² is

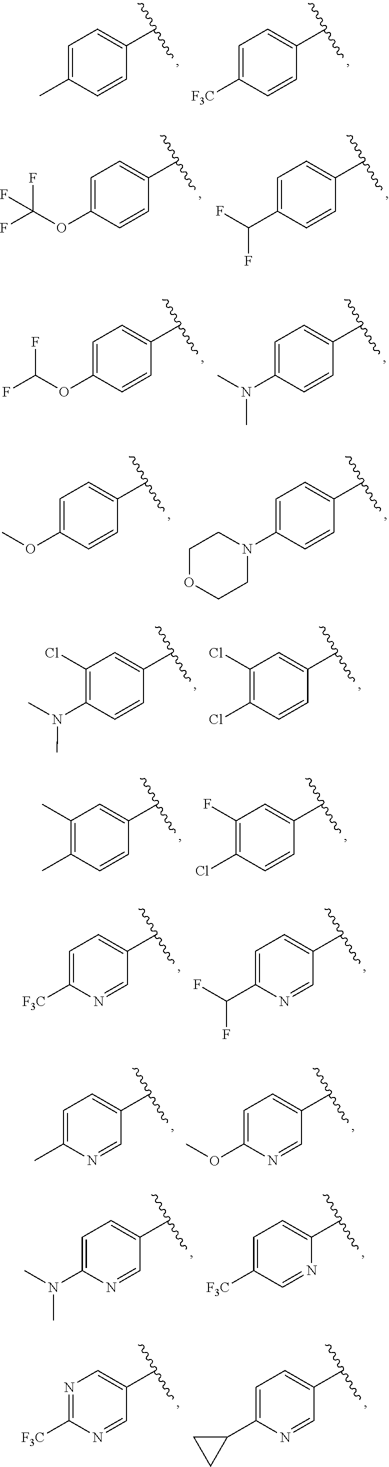

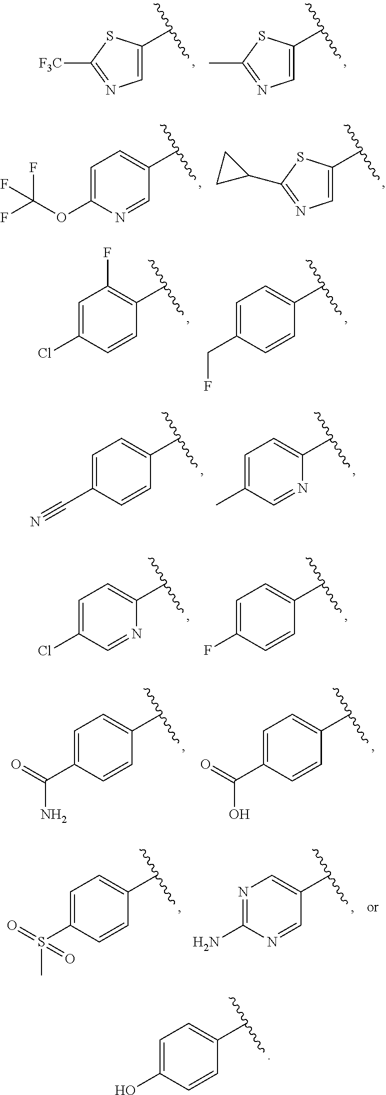

47. The compound or salt of claim 45, wherein Ar² is

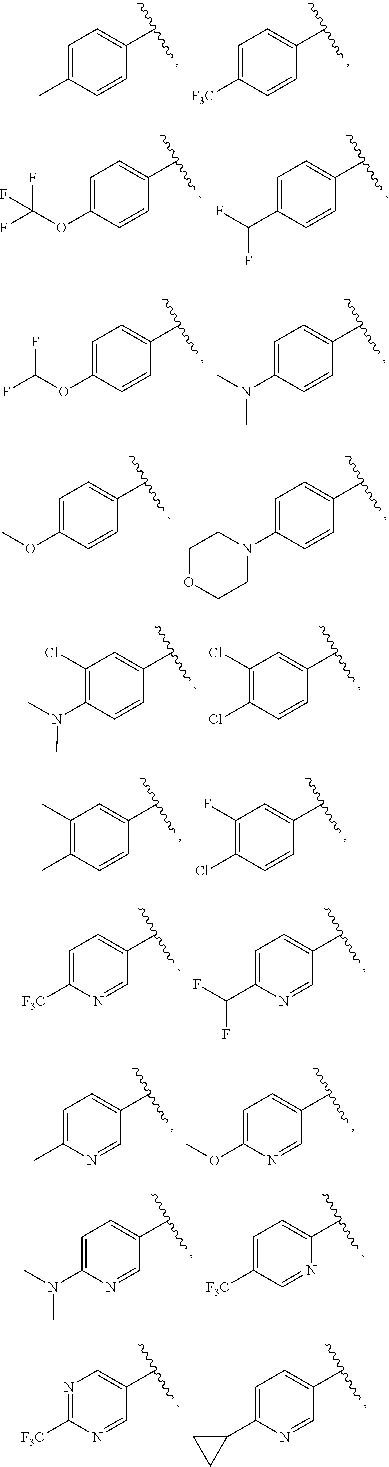

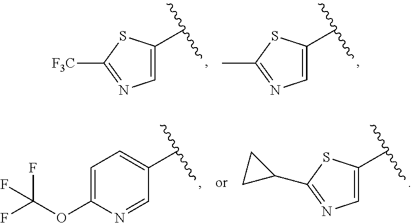

48. The compound or salt of any one of claims 1 to 31, wherein Ar² is

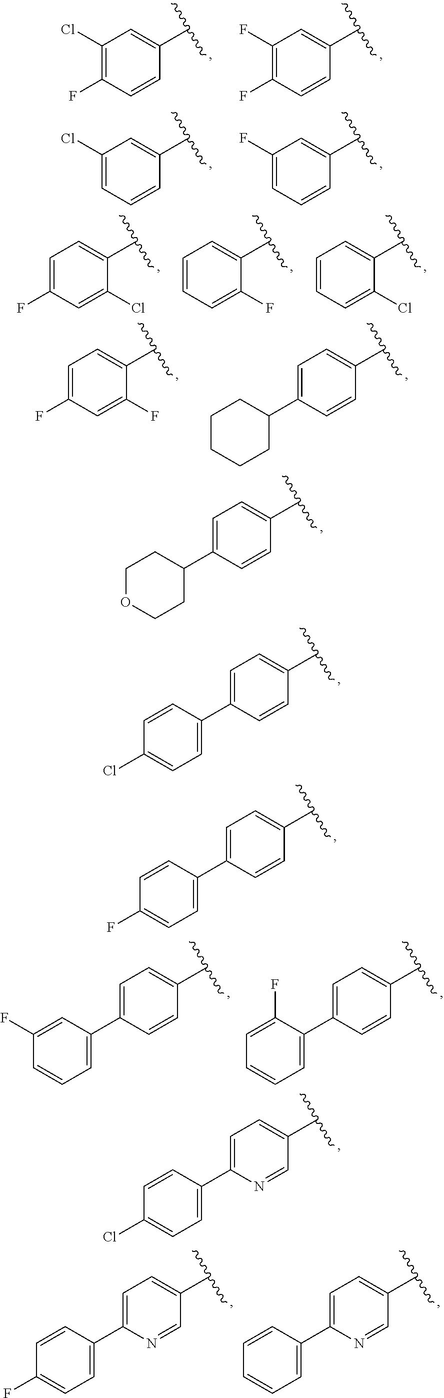

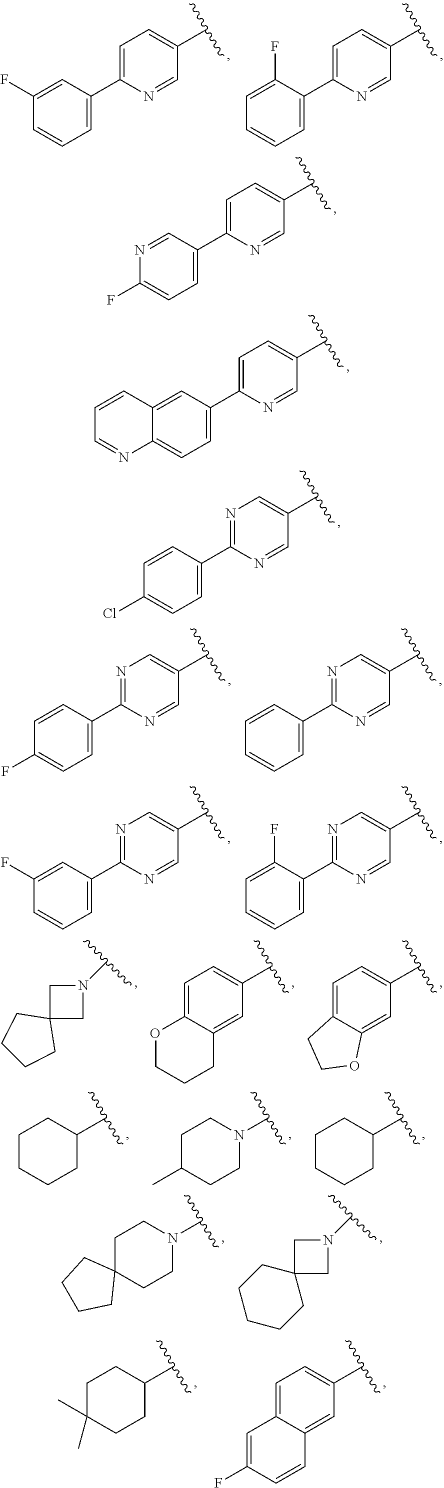

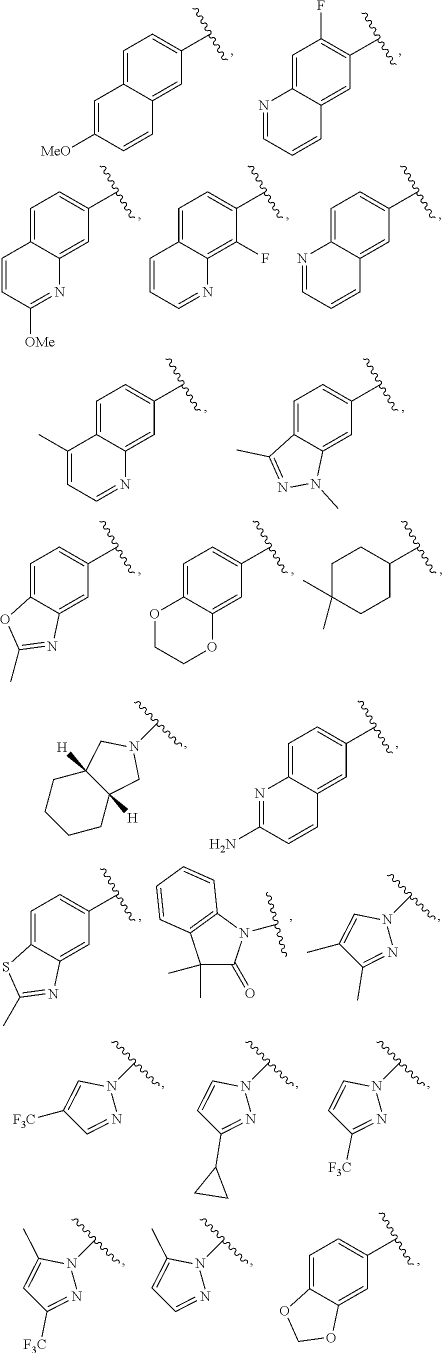

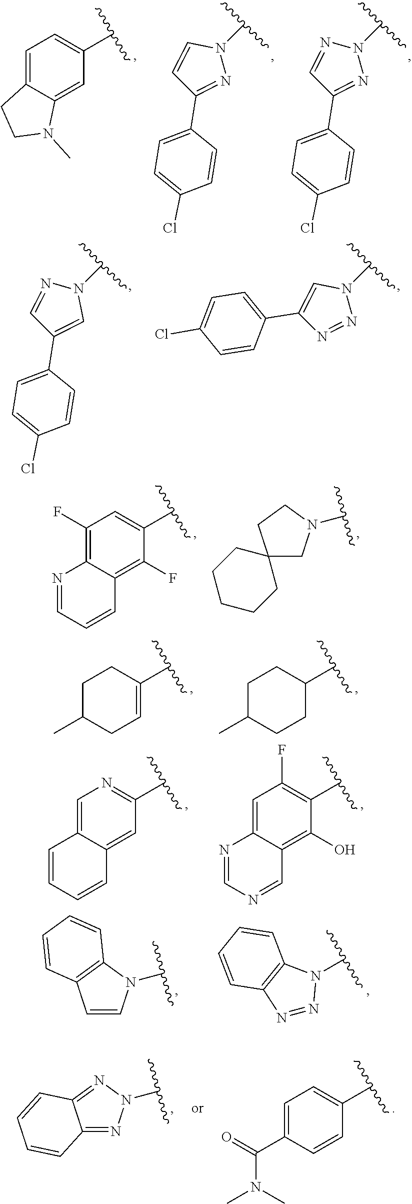

49. A compound as listed in Table 1 or a pharmaceutically acceptable salt thereof.

50. A pharmaceutical formulation comprising the compound or salt of any one of claims 1 to 49 and a pharmaceutically acceptable excipient.

51. A method for modulating aryl hydrocarbon receptor (AHR) activity in a subject, comprising contacting the AHR with the compound or salt of any one of claims 1 to 49 or the pharmaceutical composition of claim 50.

52. A method for treating or preventing a disease or disorder in a subject, comprising administering to the subject a therapeutically effective amount of the compound or salt of any one of claims 1 to 49 or the pharmaceutical composition of claim 50.

53. The method of claim 52, wherein the disease or disorder is cancer, a viral infection, or pulmonary arterial hypertension (PAH).

54. The method of claim 53, wherein the viral infection is flavivirus infection or coronavirus infection.

55. The method of claim 54, wherein the viral infection is flavivirus infection.

56. The method of claim 55, wherein the flavivirus infection is Zika virus infection.

57. The method of claim 54, wherein the viral infection is coronavirus infection.

58. The method of claim 57, wherein the coronavirus infection is severe acute respiratory syndrome (SARS), Middle East respiratory syndrome (MERS), or Coronavirus disease 2019 (COVID-19).

59. The method of claim 58, wherein the coronavirus infection is Coronavirus disease 2019 (COVID-19).

60. The method of claim 53, wherein the disease or disorder is cancer.

61. The method of claim 60, wherein the cancer is a liquid or solid tumor, hematological cancer, lymphoma, myeloma, leukemia, sarcoma, eye cancer, thyroid cancer, parathyroid cancer, neurological cancer, skin cancer, breast cancer, uterine cancer, endometrial cancer, prostate cancer, colorectal cancer, lung cancer, head and neck cancer, gastrointestinal cancer, liver cancer, pancreatic cancer, genitourinary cancer, bone cancer, renal cancer, or vascular cancer.

62. The method of claim 53, wherein the disorder is PAH.

63. The method of any one of claims 51 to 62, further comprising administering a therapeutic agent to the subject.

64. The method of claim 63, wherein the therapeutic agent is an immune checkpoint inhibitor.

65. The method of claim 64, wherein the immune checkpoint is PD1 or PDL1.

66. The method of claim 64 or 65, wherein the immune checkpoint inhibitor is pembrolizumab, nivolumad, cemiplimab, atezolizumab, dostarlimab, durvalumab, avelumab, or a combination thereof.

67. Use of the compound or salt of any one of claims 1 to 49 or the pharmaceutical composition of claim 50 for modulating AHR activity in a subject.

68. Use of the compound or salt of any one of claims 1 to 49 or the pharmaceutical composition of claim 50 for treating or preventing a disease or disorder in a subject.

69. A compound having a structure of Formula (A) or (B) 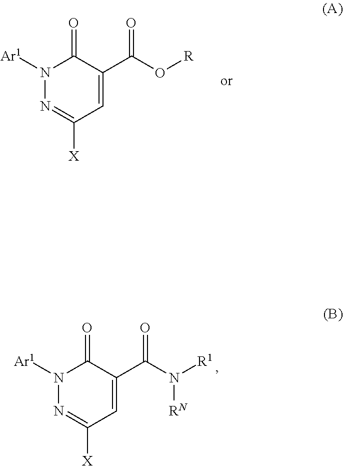
wherein
Ar¹ is C₆₋₁₀aryl or 5-10 membered heteroaryl, wherein 1-3 ring atoms are selected from O, N, and S, and Ar¹ is optionally substituted with 1 or 2 R³;
each R³ is independently halo, C₁₋₆alkyl, C₁₋₆haloalkyl, or C₃₋₆cycloalkyl;
R¹ is H, C₁₋₆alkyl, C₁₋₆alkylene-NR^{N}R^{N}, C₁₋₆alkylene-O—C₁₋₆alkyl, C₁₋₆alkylene-C(O)R², C₁₋₆alkylene-NR^{N}—C(O)—C₁₋₃alkyl, C₃₋₈cycloalkyl, or 4-12 membered heterocyclyl, wherein 1-3 ring atoms are selected from O, N, and S, and the cycloalkyl or heterocyclyl is optionally substituted with 1 or 2 C₁₋₆alkyl, C(O)—C₁₋₆alkyl, and ═O;
R² is OH, O—C₁₋₆alkyl, or NR^{N}R^{N};
each R^{N} is independently H or C₁₋₆alkyl;
X is a leaving group; and
R is H, C₁₋₆alkyl, C₁₋₆alkylenephenyl, aryl or heteroaryl, and when alkyl, aryl, or heteroaryl, can be optionally substituted.

70. The compound of claim 69, wherein X is chloro, fluoro, mesyl, tosyl, or triflyl.

71. A method of preparing the compound or salt of Formula (I) of any one of claims 1 to 49, comprising admixing the compound of Formula (B) of claim 69 or 70 with Ar²-boronic acid or Ar²-boronic ester in the presence of a catalyst to form the compound of Formula (I).

72. A method of preparing the compound or salt of Formula (I) of any one of claims 1 to 49, comprising admixing the compound of Formula (A) of claim 69 or 70 with Ar²-boronic acid or Ar²-boronic ester in the presence of a catalyst to form an intermediate then converting the —OR moiety of the intermediate to a —N(R^{N})R¹ moiety to form the compound of Formula (I).

73. The method of claim 71 or 72, wherein the catalyst comprises a palladium catalyst.

74. A method of preparing the compound or salt of Formula (I) of any one of claims 1 to 49, comprising admixing the compound of Formula (B) of claim 69 or 70 with an amine nucleophile, optionally in the presence of a catalyst, to form an aromatic intermediate, then reacting the aromatic intermediate with a reagent to form the compound of Formula (I).

75. A method of preparing the compound or salt of Formula (I) of any one of claims 1 to 49, comprising (i) admixing the compound of Formula (A) of claim 69 or 70 with an amine nucleophile, optionally in the presence of a catalyst, to form an aromatic intermediate, (ii) reacting the aromatic intermediate with a reagent to form an ester intermediate, and (iii) converting the —OR moiety of the ester intermediate to a —N(R^{N})R¹ moiety to form the compound of Formula (I).
